(19) United States
(12) Patent Application Publication
(10) Pub. No.: US 2005/0136481 A1
(43) Pub. Date: Jun. 23, 2005

(54) Systems and methods for predicting the structure and function of multipass transmembrane proteins

(76) Inventors: Rene J. Trabanino, Bell Gardens, CA (US); Nagarajan Vaidehi, Arcadia, CA (US); Spencer E. Hall, Pasadena, CA (US); William A. Goddard, Pasadena, CA (US); Wely Floriano, Pasadena, CA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/918,531

(22) Filed: Aug. 13, 2004

Related U.S. Application Data
(60) Provisional application No. 60/494,971, filed on Aug. 13, 2003.

(51) Int. Cl. G01N 33/53; G06F 19/00; G01N 33/48; G01N 33/50

(52) U.S. Cl. 435/7.1; 702/19

(57) ABSTRACT

The invention provides computer-implemented methods and apparatus implementing a hierarchical protocol using multiscale molecular dynamics and molecular modeling methods to predict the structure of transmembrane proteins such as G-Protein Coupled Receptors (GPCR), and protein structural models generated according to the protocol. The protocol features a combination of coarse grain sampling methods, such as hydrophobicity analysis, followed by coarse grain molecular dynamics and atomic level molecular dynamics, including accurate continuum solvation. Also included are energy optimization to determine the rotation of helices in the (seven-helical) TM bundle, and optimization of the helix translations along their axes and rotational optimization using hydrophobic moment of the helices, to provide a fast and accurate procedure for predicting GPCR tertiary structure.

### REFERENCE TO RELATED APPLICATIONS

This application claims the benefit of the filing date, under 35 U.S.C. 119(e), of U.S. Provisional Application No. 60/494,971, filed on Aug. 13, 2003, the entire contents of which is incorporated herein by reference.

### GOVERNMENT SUPPORT

The invention described herein was supported, in whole or in part, by Grant Nos. NIHBRGR01-GM625523, NIH-R29AI40567, NIH-HD36385, R01GM6225301, and other grants such as DAAG55-98-1-0266, from other U.S. Government Agencies including DOE (ASCI ASAP) and NSF. The U.S. Government has certain rights in this invention.

### BACKGROUND OF THE INVENTION

Proteins are linear polymers made up of 20 different naturally-occurring amino acids. The particular linear sequence of amino acid residues in a protein is said to define the protein's primary structure. In its natural environment, a protein folds into a three-dimensional structure determined by its primary structure, and by the chemical and electronic interactions is between the protein's individual amino acid constituents and the surrounding aqueous environment, which can include other biomolecules and cellular structures in addition to water.

Studies of known three-dimensional structures have led to the identification of a number of characteristic patterns that appear to be particularly stable and therefore recur within folded proteins. Formed as a result of chemical interactions between different amino acids in the protein, these patterns, which include alpha helices, beta sheets and turns, among others, are referred to as the protein's secondary structure.

The combination of α-helices, β-sheets, turns, and other structures that make up the protein's secondary structure, and the interactions between those structures, determine the protein's tertiary (or three-dimensional) structure. Because a protein's biological properties depend directly on its tertiary structure (i.e., its three-dimensional conformation), understanding that structure is crucial to understanding a protein's biological role and to applying that understanding in such areas as the treatment of disease and the design and development of new pharmaceuticals.

A protein's primary structure can be easily determined using known methods—for example, by identifying the amino acids coded for by a protein's known genetic (nucleotide) sequence. Similarly, known techniques make it relatively easy to identify a protein's secondary structure once the primary structure is determined.

Determining a protein's tertiary structure is more difficult. For some proteins, it is possible to determine tertiary structure through such techniques as x-ray crystallography, or spectroscopic methods such as fluorescence and nuclear magnetic resonance (NMR) studies. However, these techniques can be time consuming and expensive, and not all proteins are equally amenable to structural examination by these methods.

One such class of proteins is the class of transmembrane/integral membrane proteins. Integral membrane proteins comprise 20-30% of genes (Wallin and von Heijne, 1998) in humans and other forms of life, playing an important role in processes as diverse as ion translocation, electron transfer, and transduction of extracellular signals. One of the most important classes of transmembrane (TM) proteins is the G-protein-coupled receptor (GPCR) superfamily which, upon activation by extracellular signals, initiates an intracellular chemical signal cascade to transduce, propagate, and amplify these signals. GPCRs are involved in cell communication processes and in mediating such senses as vision, smell, taste, and pain. The extracellular signals inciting this transduction are usually chemical, but for the opsin family, it is visible light (electromagnetic radiation). Malfunctions in GPCRs play a role in such diseases as ulcers, allergies, migraine, anxiety, psychosis, nocturnal heartburn, hypertension, asthma, prostatic hypertrophy, congestive heart failure, Parkinson's, schizophrenia, and glaucoma (Wilson and Bergsma, 2000). Indeed, although they comprise 34% (Schöneberg et al., 2002) of the human genome, the GPCR superfamily is one of the most important families of drug targets.

GPCRs share a predicted seven-transmembrane helix structure and the ability to activate a G-protein in response to ligand binding. Their natural ligands range from peptide and non-peptide neurotransmitters, hormones, and growth factors to odorants and light. The members of the GPCR superfamily which act through heterotrimeric G-proteins have been classified into six clans (see below). Within a class of GPCRs (for example, adrenergic receptors) there are often several subtypes (for example, nine for adrenergic receptors) all responding to the same endogenous ligand (epinephrine and norepinephrine for adrenergic receptors), but having very different functions in various cells. In addition, many different types of GPCRs are similar enough that they are affected by the antagonists or agonists for other types (e.g., among adrenergic, dopamine, serotonin, and histamine receptors), leading often to undesirable side effects. This makes it difficult to develop drugs to a particular subtype without side effects resulting from cross-reactivity to other subtypes. To design such subtype-specific drugs it is essential to use structure-based methods.

Extensive protein sequence analyses on certain GPCRs has revealed a common topology consisting of a membrane-spanning seven-helix bundle, which is believed to accommodate the binding site for low molecular weight ligands. However, although much effort has been put into elucidating the structure of GPCRs, only a very small number of complete 3D structures of transmembrane proteins are known from experiments (e.g., bacteriorhodopsin and bovine rhodopsin). In fact, there is no atomic-level structure available for any human GPCRs. Consequently, design of subtype-specific drugs for GPCR targets is a very tedious empirical process, often leading to drugs with undesirable side effects.

The difficulty in obtaining three-dimensional structures for GPCRs is obtaining high-quality crystals of these membrane-bound proteins sufficient to obtain high-resolution x-ray diffraction data, and the difficulty of using NMR to determine structure on such membrane-bound systems. For globular proteins, there have been significant advances in predicting the three-dimensional structures by using sequence homologies to families of known structures (Marti-Renom et al., 2000); however, this is not practical for GPCRs, inasmuch as a high-resolution crystal structure is available for only one GPCR, bovine rhodopsin-which has low homology (<35%) to most GPCRs of pharmacological interest. Thus, there is a need for modeling techniques that predict structure and functional characteristics of the members of this class of proteins at a molecular level.

From a different perspective, since the function of a GPCR is to signal to the interior of the cell in the presence of a particular ligand bound to the extracellular surface, it is most relevant to determine the three-dimensional structure for the conformation of the protein involved in activating G-protein. It is widely thought that there are two distinct conformations of GPCRs: one active and one inactive, in equilibrium, even in the absence of ligands (Melia et al., 1997; Strange 1998; Schöneberg et al., 2002). This equilibrium is shifted when a ligand binds to the GPCR. Thus it would be valuable to know four structures of the protein—the apo-protein in both the active and inactive forms and the ligand-bound form in both the active and inactive forms-so that one could study the process of GPCR activation. However, even for bovine rhodopsin, there is crystal structure data for only one of these four (the ligand-bound inactive form). In other words, it is the closed conformation that is observed in the rhodopsin crystal structure (Palczewski et al., 2000; Okada, et al., 2001).

From yet another different perspective, except for bovine rhodopsin, the only experimental validation for the accuracy of any predicted GPCR structures must rest on predicting the binding sites and energies for various ligands, and how they are modified by various mutations. Such validation methods are also lacking in the art.

### SUMMARY OF THE INVENTION

The present invention relates to computational methods for predicting the three-dimensional structure of transmembrane proteins, and to computer-implemented apparatus for performing such computations. Related methods are disclosed in co-pending U.S. application Ser. No. 09/816,755, which is incorporated herein by reference in its entirety.

The invention provides a hierarchical protocol using multiscale molecular dynamics and molecular modeling methods to predict the structure of multipass membrane proteins, such as G-Protein Coupled Receptors (GPCRs), from primary amino acid sequence of a target protein. The protocol features a combination of coarse grain sampling methods, such as hydrophobicity analysis, followed by coarse grain molecular dynamics and atomic level molecular dynamics, including accurate continuum solvation. The methods also include energy optimization to determine the rotation of helices in the (seven-helical) TM bundle, and optimization of the helix translations along their axes and rotational optimization using hydrophobic moment of the helices, to provide a fast and accurate procedure for predicting tertiary structure.

In general, in one aspect, the invention features methods and apparatus, including computer program apparatus, implementing techniques for predicting the structure of a multipass membrane protein having a plurality of a-helical regions. The techniques can include providing an amino acid sequence for the multipass membrane protein; using the amino acid sequence to identify one or more transmembrane regions of the membrane-bound protein; constructing a set of helices for the transmembrane regions and optimizing a helix bundle configuration both translationally and rotationally for the set of helices; fine-grain re-optimizing said TM bundle in explicit lipid bilayers; constructing a plurality of interhelical loops to generate a full-atom model of the membrane-bound protein; optimizing the full-atom model using molecular dynamics simulation; and outputting a predicted structure for the transmembrane protein based on the second optimization.

In particular implementations, constructing the set of helices for the transmembrane regions can include constructing a set of canonical helices corresponding to the predicted transmembrane regions, calculating a minimum-energy configuration for each of the canonical helices, optimizing each of the canonical helices, assembling a helix bundle including each of the set of helices, and calculating a minimum-energy configuration for the helix bundle in a lipid bilayer.

In general, in another aspect, the invention features additional methods and apparatus, including computer program apparatus, implementing techniques for modeling the structure of a transmembrane protein having a plurality of a-helical regions. The techniques can include providing amino acid sequence information and sequence alignment information for a transmembrane protein having a plurality of a-helical regions; using the amino acid sequence information and the sequence alignment information to predict a set of transmembrane segments of the transmembrane protein; constructing canonical helices for the predicted transmembrane segments and optimizing the canonical helices; combining the optimized helices based on the sequence alignment information to form a helix bundle, and assembling the helix bundle with a lipid bilayer to form a system helix bundle; optimizing the structure of the system helix bundle; adding inter-helical loops to the system helix bundle to form a full atom model; optimizing the full atom model; and outputting a predicted structure for the transmembrane protein based on the optimization.

Particular implementations of the invention can include one or more of the following features.

One aspect of the invention provides a method for predicting the structure of a transmembrane (TM) protein, comprising: (1) identifying one or more TM regions from analysis of the primary sequence of said TM protein; (2) assembling a TM bundle comprising all TM regions identified in (1), and optimizing the relative translation and/or rotational orientation of the TM regions in said TM bundle; (3) optimizing each individual TM region in said TM bundle; (4) fine-grain re-optimizing said TM bundle in explicit lipid bilayers; (5) adding inter-TM region loops and side-chains for all amino acid residues to yield a full-atom model of said TM protein; (6) optimizing said full-atom model and outputting a predicted structure therefor.

In one embodiment, the structure is a three-dimensional (3D) structure of the TM region(s) of said TM protein.

In one embodiment, the TM protein is a multipass TM protein.

In one embodiment, the multipass TM protein is an ion channel, a transporter, or a pump.

In one embodiment, the multipass TM protein has two or more TM regions.

In one embodiment, the multipass TM protein is a seven-TM protein.

In one embodiment, the seven-TM protein is a G Protein-Coupled Receptor (GPCR).

In one embodiment, the GPCR is an orphan GPCR or an olfactory receptor (OR).

In one embodiment, the multipass TM protein is a seven-TM protein, such as a G Protein-Coupled Receptor (GPCR). The GPCR may be an orphan GPCR or any other GPCR like olfactory receptor (OR). In some embodiments, the GPCR is a rhodopsin-like receptor selected from: olfactory receptor, adenosine receptor, melanocortin receptor, biogenic amine receptor, vertebrate opsin, neuropeptide receptor, prostaglandin receptor, prostacyclin receptor, thromboxane receptor, lipid and peptide receptor, invertebrate opsin, chemokine receptor, chemotactic receptor, somatostatin receptor, opioid receptor, or melatonin receptor; a calcitonin or related receptor selected from: calcitonin receptor, calcitonin-like receptor, CRF receptor, PTH/PTHrP receptor, glucagon receptor, secretin receptor, or latrotoxin receptor; a metabotropic glutamate or related receptor selected from: metabotropic glutamate receptor, calcium receptor, GABA-B receptor, or putative pheromone receptor; a STE2 pheromone receptor; a STE3 pheromone receptor; vermonasal organ (VNO) receptor, or a cAMP receptor.

In one embodiment, the TM region comprises one or more potential α-helical region(s).

In one embodiment, the TM regions are TM helix regions.

In one embodiment, each of said TM helix region(s) comprises at least about 21 amino acid residues.

In one embodiment, step (1) is effectuated by a method based on hydrophobic profiling of at least a portion of said TM protein.

In one embodiment, the portion substantially excludes one or more of: the N- or C-terminal region(s) not in contact with lipid bilayers, or inter-TM region loops.

In one embodiment, the hydrophobic profiling uses peak signal analysis.

In one embodiment, the hydrophobic profiling uses the Eisenberg hydrophobicity scale.

In one embodiment, the TM regions are TM helix regions, said hydrophobic profiling uses the SeqHyd profile algorithm, and step (1) is effectuated by: (1a) using the sequence of said protein as query, retrieving from a database an ensemble of hit sequences with 20-90% sequence identity, and/or BLAST bit score>200 and E-value>e-100; (1b) obtaining a multiple sequence alignment of said hit sequences and the sequence of said protein; (1c) calculating consensus hydrophobicity for every residue position in said alignment, and plotting a hydrophobic profile; (1d) assigning initial TM helix regions based on the global average hydrophobicity of said alignment, or a base_mod within 0.05 thereof; (1e) capping each initial TM helix region identified in (1d) to yield a capped TM helix region, based on the presence of helix breakers.

In one embodiment, the database is a protein database, or a polynucleotide database translated in at least one of the six reading frames.

In one embodiment, the ensemble of hit sequences have a uniform distribution of sequences over the entire range of sequence identities.

In one embodiment, the lowest sequence identity to said protein within said ensemble of sequences is about 20-40%, preferably 40%.

In one embodiment, the multiple sequence alignment is performed with ClustalW.

In one embodiment, step (1c) is performed with a window size (WS) between 12-20.

In one embodiment, step (1d) further comprising identifying additional TM region(s) with peak length <23 and peak area <0.8, using local average hydrophobicity more than 0.05 less than said base_mod, if said additional TM region(s) are not identified based either on said global average hydrophobicity or said base_mod.

In one embodiment, the helix breakers are independently selected from Pro (P), Gly (G), Arg (R), His (H), Lys (K), Glu (E), or Asp (D).

In one embodiment, the capped TM helix regions exclude N- and C-terminal helix breakers.

In one embodiment, the N- and C-termini of said capped TM helix regions are no more than 6 residues longer or 4 residues shorter than the N- and C-termini of said initial TM helix regions.

In one embodiment, each residue in each of said capped TM helix regions has an α-helical conformation.

In one embodiment, the α-helical conformation is characterized by a φ between −37 and −77 degrees, and a ψ between −27 and −67 degrees.

In one embodiment, the α-helical conformation is checked by verifying φ and ψ using PROCHECK.

In one embodiment, the protein is a GPCR, and in step (2), said TM bundle is assembled based on the 7.5 Å electron density map of frog rhodopsin.

In one embodiment, the protein is a GPCR, and in step (2), the relative translation of all helices in said TM bundle is optimized by placing the hydrophobicity center (HC) of each helix in the lipid midpoint plane (LMP).

In one embodiment, the protein is a GPCR, and in step (2), the rotational orientation of at least one helix in said TM bundle is optimized by the hydrophobic moment-based phobic orientation/CoarseRot-H.

In one embodiment, the phobic orientation depends on a hydrophobic midregion (HMR) defined by the middle 15 residues of each helix straddling the predicted HC.

In one embodiment, the at least one helix has significant contacts with a lipid membrane straddling the LMP.

In one embodiment, the protein is a GPCR, and in step (2), the rotational orientation of each of the at least one helix in said TM bundle is optimized individually by the energy minimization process RotMin.

In one embodiment, for each of the at least one helix, the RotMin comprises: (a) designating one of said at least one helix as active helix; (b) keeping the main chain of said active helix rigid while rotating said main-chain for a grid of rotation angles; (c) optimizing side-chain positions of all residues for all helices in said TM bundle; (d) minimizing energy for said active helix in the field of all other helices; (e) repeating (a)-(d) for each of said at least one helix.

In one embodiment, the grid of rotation angles comprises a change of 2.5, 5, or 8 degrees over a range of +50 to +100 degrees.

In one embodiment, (c) is effectuated using SCWRL.

In one embodiment, (d) is effectuated by conjugate gradient minimization until an RMS force of 0.5 kcal/mol per Å is achieved.

In one embodiment, the at least one helix is near the center of the GPCR TM barrel and not strongly amphipathic.

In one embodiment, the protein is a GPCR, and in step (2), the rotational orientation of all helices in said TM bundle is optimized by a combination of CoarseRot-H and RotMin.

In one embodiment, step (2) comprises generating an ensemble of assembled TM bundles by randomly combining permutations of favorable conformations of each TM region within the bundle, and screening for the most favored assembled TM bundle.

In one embodiment, the method further comprises repeating the translation and/or rotational optimization in step (2) immediately after step (3).

In one embodiment, for each individual TM region, step (3) is effectuated by: (a) placing all side-chains; (b) minimizing energy using molecular dynamics (MD).

In one embodiment, step (a) is effectuated by SCWRL.

In one embodiment, step (b) is effectuated by simulations at 300 K for 500 ps, and minimizing the structure with the lowest potential energy in the last 250 ps using conjugate gradients.

In one embodiment, the molecular dynamics is Cartesian or torsional MD/NEIMO.

In one embodiment, the explicit lipid bilayers comprise molecules of dilauroylphosphatidylcholine lipid.

In one embodiment, step (4) is effectuated by quaterion-based rigid-body molecular dynamics (RB-MD) in MPSim.

In one embodiment, the MPSim is carried out for 50 ps or until the potential and kinetic energies of the system are stabilized.

In one embodiment, the loops are added in step (5) by WHATIF, and the side-chains for all amino acid residues are added by SCWRL.

In one embodiment, in step (5), the conformations of the loops are optimized by conjugate gradient minimization while keeping all TM regions fixed.

In one embodiment, step (5) further comprises adding selected disulfide bonds.

In one embodiment, step (6) is effectuated by full-atom conjugate gradient minimization in vacuum using MPSim.

In one embodiment, the method further comprises verifying the predicted struture using HierDock.

Another aspect of the invention provides a method for identifying a ligand specifically binding a GPCR, comprising: (1) predicting the struture of said GPCR using the subject method; (2) identifying, amongst a plurality of candidate ligands, one or more ligands, if any, that specifically bind said GPCR using HierDock; (3) verifying the binding specificity of each said one or more ligands to one or more other GPCRs; wherein a preferential binding by said one or more ligands to said GPCR over said other GPCRs is indicative that said one or more ligands bind specifically to said GPCR.

In one embodiment, the GPCR is a target for a disease or condition.

In one embodiment, the GPCR is a mutant protein associate with a disease or condition.

In one embodiment, the ligand is an agonist of said GPCR.

In one embodiment, the ligand is an antagonist of said GPCR.

In one embodiment, step (3) is effectuated by HierDock.

In one embodiment, the one or more ligands bind to said GPCR with a minimal binding energy at least about 5-10 kcal/mol less than that for binding to said other GPCRs.

In one embodiment, step (3) is effectuated by biochemical measuring of ligand-receptor binding constant K_{D}.

In one embodiment, the one or more ligands bind to said GPCR with a KD at least about 10-fold lower than that for binding to said other GPCRs.

Another aspect of the invention provides a method to predict the binding site of a ligand to a target protein, the method comprising: (1) scanning at least a selected portion of the entire surface of a model of the target protein for energetically preferred docking regions for said ligand; (2) coarse-grain sampling, in each docking region identified in (1), a first plurality of ligand conformations as nonflexible molecules to locate the most favorable docking region(s) for said ligands; (3) fine-grain sampling, in the most favorable docking region identified in (2), a second plurality of ligand conformations as nonflexible molecules to identify the best ligand binding conformations; (4) energy-minimizing and selecting for the best ligand binding conformations identified in (3) by conjugate-gradient with all atoms in said target protein fixed; (5) using conjugate gradients, energy-minimizing a complex of said target protein with bound best conformation ligand, with all atoms of said protein, said best conformation ligand, and any lipid solvent movable; (6) for the best binding conformation in (5), optimizing side-chain conformations for all target protein residues within 5 A of the bound ligand; thereby predicting the binding site occupied by said best conformation bound ligand as said binding site for said ligand in said target protein.

In one embodiment, the method further comprises: (7) repeating (3)-(6) one or more times to obtain the best possible conformation for the ligand bound within the target protein.

In one embodiment, the target protein is a multipass transmembrane protein.

In one embodiment, the multipass transmembrane protein is a GPCR.

In one embodiment, the model is predicted using the subject method, such as MembStruk3.5.

In one embodiment, the selected portion excludes portions of said target protein known not to bind said ligand.

In one embodiment, step (1) is effectuated by the Sphgen program in DOCK.

In one embodiment, the first plurality of ligand conformations comprise about 100 ligand conformations.

In one embodiment, the second plurality of ligand conformations comprise about 1000 ligand conformations.

Another aspect of the invention provides a computer executable software code stored in a computer readable medium, which upon execution carries out a method for predicting the structure of a transmembrane (TM) protein, said method comprising: (1) identifying one or more TM regions from analysis of the primary sequence of said TM protein; (2) assembling a TM bundle comprising all identified TM regions, and optimizing the relative translation and/or rotational orientation of the TM regions in said TM bundle; (3) optimizing each individual TM region in said TM bundle; (4) fine-grain re-optimizing said TM bundle in explicit lipid bilayers; (5) adding inter-TM region loops and side-chains for all amino acid residues to yield a full-atom model of said TM protein; (6) optimizing said full-atom model and outputting a predicted structure therefor.

Another aspect of the invention provides a system for predicting the structure of a transmembrane (TM) protein, comprising: (1) a TM region identification module for identifying one or more TM regions from analysis of the primary sequence of said TM protein; (2) a bundle assembly module for assembling a TM bundle comprising all TM regions identified in (1), and optimizing the relative translation and/or rotational orientation of the TM regions in said TM bundle; (3) a TM region optimization module for optimizing each individual TM region in said TM bundle; (4) a fine grain re-optimization module for fine-grain re-optimizing said TM bundle in explicit lipid bilayers; (5) a full-atom model generation module for adding inter-TM region loops and side-chains for all amino acid residues to yield a full-atom model of said TM protein; (6) a full-atom optimization module for optimizing said full-atom model and outputting a predicted structure therefor.

Another aspect of the invention provides a system for predicting a three-dimensional structure of a transmembrane (TM) protein having one or more potential α-helical region(s), the system comprising: (1) a computer executable program for predicting three-dimensional structure according to the method of claim 1, said program being stored on a computer readable storage medium or in a ROM of a (Central Processing Unit) CPU; (2) an instruction for installation of the program on a computer, and/or using said program for predicting a three-dimensional structure.

In one embodiment, the system further comprises a computer executable program for predicting binding site of a ligand to said TM protein using the subject method, such as HierDock.

In one embodiment, the system further comprises a computer with said program and/or said instruction installed.

Another aspect of the invention provides a computational model of the structure of a transmembrane protein, the computational model comprising: a computer-readable memory storing data describing an optimized predicted three-dimensional structure for the transmembrane protein, the optimized predicted structure being generated according to the subject method, such as MembStruk3.5.

In one embodiment, the computational models can include computer readable data storage media storing data describing a predicted three-dimensional structure for such proteins, including, for example, olfactory receptors S6, S18r, S19f, S25r, S46, or S50.

In some embodiments of the invention, the molecular dynamics for calculations such as energy minimization can include mixed mode molecular dynamics simulations. At least the last-simulation can preferably include solvent approximations, such as a continuum solvation model or empirical solvation model based on estimating solvation free energy based on solvent-accessible protein surface area. Some examples of appropriate solvent models include the Surface Generalized Born (SGB) model or a Poisson-Boltzmann (PB) description model.

In one embodiment, the predicted structure can be generated by performing the last molecular dynamics simulation for a time in the range from about 100 ps to about 1 ns.

It is contemplated that all embodiments described above, even embodiments under different aspects of the inventions, are to be freely combined with any one or more other embodiments of the invention whenever appropriate.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 Hydrophobicity profile from TM2ndS for bovine rhodopsin at window size of WS=14. The pink line (at 0.07) is the base_mod (described in Step 1, average consensus hydrophobicity and initial TM assignment) used as the baseline in identifying hydrophobic regions. The predicted TM domains are indicated by the orange lines (after capping). The blue lines show the predictions before helix capping. Each tick mark indicates the sequence number for the alignment based on bovine rhodopsin (100 residues per panel). The residues at every fifth position are indicated below each panel. The partition of helix 7 into two parts results from the hydrophilic residues near its center.

FIG. 2 The transmembrane helical predictions (labeled as after capping) from TM2ndS compared with helix ranges from the bovine rhodopsin x-ray crystal structure. The predictions before TM2ndS capping are also shown. Those residues in the crystal structure that fall outside the range of α-helicity (using analysis described in Step 1c, Helix Capping in TM2ndS) are indicated in lowercase letters. Sequences in this figure is represented by SEQ ID NOs: 1-20.

FIG. 3 The RMS deviation for various window sizes (WS) of the central residues predicted from TM2ndS for bovine rhodopsin compared to the best-fit plane to the crystal structure minimized without ligand, Apo/closed(xtal). This suggests that the best WS is 16-22.

FIG. 4 Schematic for a possible signaling mechanism in rhodopsin. Note that the movement of helix 3 (caused by interaction with the trans-isomer of retinal) exposes the DRY sequence to G-protein activation and as a result closes the EC-II loop to maintain the ligand inside the bundle sequence.

FIG. 5 The 13 regions shown as boxes used in scanning the entire protein for the 1 1-cis-retinal putative binding site. The two boxes chosen as binding sites by HierDock are shown in red. (A) Front view with N-terminus at the bottom. (B) Top view obtained by rotating by 90° around the horizontal axis in A so that the N-terminus is out of view. These two orientations are used for all structures shown in this article.

FIG. 6(A-D) Validation of HierDock. (A) Front view of the 1 1-cis-retinal conformation determined by HierDock for Ret(HD)/closed(xray) (colored by element) compared to the published crystal structure (green). The CRMS difference in the ligand structures is 1.1 Å. (B) Top view of A. This shows that predicted position of the retinal aldehyde oxygen is 2.8 Å from the N of Lys-296, which is short enough for an H-bond. (C) Top view showing the result of making the Schiff base bond of 11-cis-retinal to Lys-296 in A and minimizing the resulting structure (blue), compared with the crystallographic ligand structure (red). The CRMS difference between these ligand structures is 0.62 Å. (D) Top view of C.

FIG. 7(A) Comparison of the predicted structure (orange) Apo/closed(MS) with the experimental structure (blue) Apo/closed(xray). They differ in the TM helical region by CRMS=2.84 Å. (B) Comparison of the predicted Apo/closed(MS) structure (orange) with the predicted Apo/open(MS) structure (blue). They differ in the TM helical region by 0.11 Å.

FIG. 8 MembStruk validation using the closed EC-II loop. (A) The HierDock-predicted conformation of 11-cis-retinal (colored by element) in the MembStruk-predicted Apo/closed(MS) structure, denoted NoSB-Ret(HD)/closed(MS). Note that the aldehyde oxygen is 2.85 Å from the N of Lys-296. (B) The retinal structure after forming this Schiff base bond of 11-cis-retinal to Lys-296 and optimizing to form Ret(HD)/closed(MS) (violet). Ret(HD)/closed(xtal) (blue). These ligand structures were found to differ by 2.9 Å CRMS.

FIG. 9(A-D) Comparison of predicted binding sites for retinal (those residues within 5 Å of retinal that interact strongly with the ligand (contributions to binding>1 kcal/mol) before Schiff base bond formation in the three rhodopsin structures. (A) All three structures and ligand conformations are shown. The colors blue, gray, and orange correspond respectively to those structures analyzed in B-D. (B) NoSB-Ret(HD)/closed(xtal) structure. Here, seven residues bind more strongly than 1 kcal/mol. (C) NoSB-Ret(HD)/closed(MS). Here, five of the seven residues in B are predicted (only Phe-208 and Hsp-211, both rather weakly bound). Three additional residues (Phe-212, Ile-275, and Ala-117) do not bind with 1 kcal/mol in B. (D) NoSB-Ret(HD)/open(MS). Here, six of the seven residues in C bind more strongly than 1 kcal/mol. Four additional residues do not bind with 1 kcal/mol in B. This difference results from the shift in the retinal binding site upon closure of the EC-II loop. The side chains in common with the NoSB-Ret(HD)/closed(xtal) structure (in B) or with NoSB-Ret(HD)/closed(MS) (in C) within the binding site around the 11-cis-retinal are labeled with larger type.

FIG. 10(A-C) Comparison of predicted binding sites of retinal with Schiff base bond formed. Residues within a 5 A shell of the ligand (excluding the Lys-296 to which the retinal is bound) are considered, and those that contribute at least 1 kcal/mol of stabilization energy for the three rhodopsin structures are determined. (A) Ret(xtal)/closed(xtal) structure. Here, 15 residues bind more strongly than 1 kcal/mol. (B) Ret(HD)/closed(xtal). Here, 12 of the 15 residues in A are predicted to bind strongly (Ala-117 and His-211 still contribute positively to bonding but are now rather weakly bound, at <1 kcal/mol). Two additional residues (Cys-187 and Ala-269) did not bind with 1 kcal/mol in A. (C) Ret(HD)/closed(MS). Here, 8 of the 15 residues in A still bind strongly. Two additional residues (Ile-275 and Ala-269) did not bind with 1 kcal/mol in A. A larger type is used to label the side chains in common with the Ret(xtal)/closed(xtal) structure within the binding site around the 11-cis-retinal.

FIG. 11 MembStruk validation using the open EC-II loop. (A) The HierDock-predicted conformation (colored by element) of 11-cis-retinal in the MembStruk-predicted structure to form the NoSB-Ret(HD)/open(MS) structure. Note that the aldehyde oxygen is 2.87 Å from the N of Lys-296, which is short enough to form a hydrogen bond. (B) The Ret(HD)/open(MS) structure after forming the Schiff base bond (green), compared with the structure (violet) of 11-cis-retinal in Ret(HD)/closed(MS). These ligand structures differ by 1.7 Å CRMS. The EC-II loop may function to position the retinal ligand into its final conformation as found in the rhodopsin crystal structure.

FIG. 12 The sequences (43 Blast entries shown) used to generate the multiple sequence alignment with bovine rhodopsin.

### DETAILED DESCRIPTION OF THE INVENTION

I. Overview

The methods, computer programs, and apparatus of the invention provide a system to aid the theoretical methods adequate for ab initio or first principles prediction of the three-dimensional (3D) structures of transmemebrane proteins, including multipass membrane proteins such as GPCRs, from amino acid sequence of a target protein. In certain embodiments of the invention, the system of the invention can aid the structure-based drug design for GPCR targets.

In one preferred embodiment, the system of the invention is the MembStruk suite of methods for first principles prediction of three-dimensional structures for multipass membrane proteins (e.g. GPCRs) from primary sequence, without using homology modeling. The most recent version of MembStruk (v. 3.5) is described in detail in Trabanino et al., Biophysical Journal 86: 1904-1921, 2004, the entire content of which is incorporated herein by reference.

The methods/system of the invention is partly based on the organizing principle provided by knowing that a multipass membrane protein has a single polypeptide chain with multiple (usually more than 2-3, preferably seven) TM domains threading through the membrane. Such structural feature provides sufficient structural information (when combined with atomistic simulations such as molecular dynamics and Monte Carlo) for the methods of the invention to deduce three-dimensional structures for these transmembrane proteins (e.g. GPCRs) that are adequate for prediction of the ligand (e.g. agonists and antagonists) binding site, and relative binding energy of agonists and antagonists.

The methods of the invention are validated in the Examples below, by applying the method to several GPCRs, where the validation has been based on the comparison of the predicted binding site to experimental binding and mutation data, including the only high-resolution crystal structure available for a GPCR, bovine rhodopsin.

In another aspect, the methods/system of the invention are used to predicted structures for multiple rhodopsin structures (open or closed, with or without bound retinoid, etc.), although comparison can be made directly to experiment only for the closed-loop-with-ligand case. In predicting these rhodopsin activation models involving the second extracellular (EC-II) loop and TM3, the structure is assumed: 1) to be in the active form when the EC-II loop is open, and, 2) to be in the inactive form when the EC-II loop is closed.

In another aspect, to make sure that predictions from first principles are reasonably accurate and useful for a variety of purposes, the invention also provides the HierDock method, which is validated in the Examples below, by predicting the binding site of retinal in bovine rhodopsin, both for the experimental three-dimensional structure and for the predicted structures (open and closed loop). Results in these examples indicate that the HierDock protocol is generally applicable to ligand-protein docking without prior knowledge of binding sites.

Details of the different embodiments of the invention, including the 3D structure prediction methods exemplified by MembStruk, and the verification methods exemplified by the HierDock protocol, are described below.

The present invention provides a computational hierarchical strategy for predicting the structure of certain transmembrane proteins such as multipass membrane proteins as exemplified by G-protein coupled receptors (GPCRs). In brief, starting with the amino acid sequence for a multipass membrane protein (obtained, for example, from public or commercial databases such as GenBank, EMBL, DDBJ, etc.), the protein structure prediction strategy predicts the transmembrane (TM) regions/domains for the protein. The method then performs coarse grain modeling of the predicted transmembrane regions. The method concludes with fine grain modeling of the whole protein to yield a three-dimensional model of the transmembrane protein.

The techniques described herein can be implemented using a modeling system. The modeling system of the invention includes a general-purpose programmable digital computer system of conventional construction, including a memory and a processor for running a modeling program or programs. The modeling system may also include input/output devices, and, optionally, conventional communications hardware and software by which computer system can be connected to other computer systems.

In some embodiments, the modeling system of the invention can be implemented on a single computer system. In a related embodiment, the functions of the model system of the invention can be distributed across multiple computer systems, such as on a network. Those skilled in the art will recognize that the system of the invention can be implemented in a variety of ways using known computer hardware and software, such as, for example, a Silicon Graphics Origin 2000 server having multiple R10000 processors running at 195 MHz, each having 4 MB secondary cache, or a dual processor Dell PowerEdge system equipped with Intel PentiumIII 866 MHz processors with 1 Gb of memory and a 133 MHz front side bus. More advanced and/or powerful systems are constantly being produced, and are all commercially available.

In some embodiments, the steps of the subject method can be implemented by a computer system comprising modules, each adapted to perform one or more of the steps. Each module can be implemented either independently or in combination with one or more of the other modules. A module can be implemented in hardware in the form of a DSP, ASP, Reprogrammable ROM device, or any other form of integrated circuit, in software executable on a general or special purpose computing device, or in a combination of hardware and software.

In general, the method starts with an amino acid sequence obtained from memory or some other source, such as a commercial database as discussed above (e.g. from internet). The sequence information is used to identify transmembrane regions (see below for detail). In one embodiment, transmembrane regions are identified on the basis of an amino acid hydrophobicity profile using a TM2ndS-like program or module (see below).

Alternatively, transmembrane regions are identified using the multisequence profile method of Donnelly, D. (1993) Biochem. Soc. Trans. 21, 36-39 (implemented, e.g., in PERSCAN), which is incorporated by reference herein. Sequences are aligned in the latter by the iterative profile alignment utility of WHATIF, according to Vriend, G. (1990) J. Mol. Graphics 8, 52-56, which is incorporated by reference herein. The output from this step is a range or ranges of amino acids in the sequence that are predicted to be in the transmembrane region.

This information is then used to construct TM regions, such as canonical right-handed α-helices, using known secondary structure modeling techniques, such as PolyGraf, Builder and/or Homology software applications of Accelrys (formerly Molecular Simulations, Inc.), of San Diego, Calif. These helices can then be optimized, e.g., using the Newton-Euler Inverse Mass Operator torsional MD method as described in Jain, A. et al. (1993) J. Comp. Phys. 106, 258-268; Mathiowetz, A. M., et al. (1994) Proteins 20, 227-247; and Vaidehi N., et al. (1996) J. Phys. Chem. 100, 10508-10517, each incorporated by reference herein by entirety. The output from this optimization step is a set of 3-D coordinates for the final optimized structure for each TM region (e.g. helix).

To assemble a TM helix bundle, helical translations and rotations along the helical axis, and the orientation of each helical axis are predicted. For example, using the bovine rhodopsin 7.5 Å electron density map, according to Schertler, G. F. X. (1988) Eye 12, 504-510, which is hereby incorporated herein—to obtain x and y coordinates as well as tilt for each helix. The helical axes orientation can also be incorporated from the 2.8 Å structure of Palczewski et al (2000) Science, 289, 739-745. In one embodiment, helical z-coordinates are set such that the midpoint of each helical axis is positioned in the same z-plane (e.g. lipid midpoint plane or LMP) of the assembly. Likewise, lipid-accessible residues, identified from sequence alignments and from analysis of the periodicity of hydrophobic residues in the sequence, can be oriented to face the outside of the helix barrel. To further optimize the packing of the helix bundle, the effect of the environment of the multipass membrane protein is simulated with a continuum description of the water environment and a lipid bilayer to simulate membrane environment.

This combined system is then optimized using rigid body dynamics—e.g., using the DREIDING force field with polar group charges derived from charge equilibration to simulate the lipids and charges from CHARMM22 for the protein, according to Mayo, S. L., et al. (1990) J. Phys. Chem. 94, 8897-8909, Rappe, A. K., et al. (1991) J. Phys. Chem. 95, 3358-3363, and MacKerell, A. D., et al. (1998) J. Phys. Chem. B 102, 3586-4616, each of which is incorporated by reference herein.

In one experiment, the performance of this combination of charges and parameters was evaluated through a series of constant temperature and pressure MD simulations of crystals. The systems 1,2-dilauroyl-DL-phosphatidyl ethanolamine acetic acid, disodium P-glycerophosphate hydrate, and L-a-glycerol phosphorylcholine were chosen for simulation to evaluate the performance of the force field and atomic charges progressively, from a simple polar head group to a crystal lipid bilayer. Comparison of the results of these simulations with experimental data and other simulation results available in the literature showed that the choice of charges and force field gives densities and cell parameters with less than 4% error from the experimentally determined parameters.

The rigid body dynamics is preferably done using the quaternion-based RB-MD in MPSim (Lim et al, 1997, incorporated herein by reference) for 50-150 ps, or until the potential and kinetic energies of the system stabilized).

Loops are then added to the helices using known techniques. In one embodiment, loops are added using the WHATIF software referred to above, although any comparable loop-building software, including commercially available software could be used. Side-chains are added by SCWRL or other equivalent software. Disulfide bonds can also be added at this stage.

After the addition of loops, a full atom minimization of the complete model with a barrel of lipid surrounding the protein is performed, followed by dynamic optimization of the structure by using the Massively Parallel Simulation program (MPSim), according to Lim, K. T., et al., J. Comput. Chem. 18, 501-521 (1997), which is incorporated by reference herein. The MPSim software implements molecular dynamics techniques such as: (i) the cell multipole method for fast and accurate calculation of nonbond forces, according to Ding, H. Q. et al. (1992) J. Chem. Phys. 97, 4309-4315, and Ding, H. Q., et al. (1992) Chem. Phys. Lett. 196, 6-10, both of which are incorporated by reference herein; (ii) fast torsional dynamic methods such as Newton-Euler Inverse Mass operator, according to Jain, A. et al. (1993) J. Comp. Physiol. 106, 258-268; Mathiowetz, A. M., et al. (1994) Proteins 20, 227-247; and Vaidehi, N., et al. (1996) J. Phys. Chem. 100, 10508-10517, incorporated by reference above, and Hierarchical Newton-Euler Inverse Mass Operator, according to Vaidehi, N., et al. (2000) J. Phys. Chem. 104, 2375-2383, which is incorporated by reference herein; (iii) continuum solvation techniques such as the Poisson-Boltzmann description, according to Tannor, D. J., et al. (1994) J. Am. Chem. Soc. 116, 11875-11882, which is incorporated by reference herein, and surface-generalized Born model that account for solvation in biological systems, according to Ghosh, A., et al. (1998) J. Phys. Chem B. 102, 10983-10990, which is incorporated by reference herein. Those skilled in the art will recognize that other solvation models can be used—for example, empirical solvation models that estimate solvation free energies as a function of solvent accessible surface area of the protein, as described in Williams, R. L., et al. (1992) Proteins: Structure, Function and Genetics 14, 110-119, which is incorporated by reference herein.

In one embodiment, the solution structure is optimized by performing mixed mode dynamics using the following descriptions. The helices and loops in the protein are modeled with the Newton-Euler Inverse Mass Operator torsional molecular dynamics. The lipids are treated as rigid bodies, and the counterions Na⁺ and Cl⁻ as free Cartesian atoms. Constant temperature dynamics using the Hoover algorithm is performed for 50 ps with time steps of 1 and 5 fs. The outside of the lipid layer is simulated with surface-generalized Born model continuum solvent description. A low dielectric constant of 60.0 is used to simulate the low dielectric region surrounding the membrane.

Those skilled in the art will recognize that the decision when to terminate the molecular dynamics optimization (and thereby the decision whether a particular calculated structure is a “final” structure for the purposes of the following step of the method) is to some extent up to the user, and for particular implementations may depend on such factors as computing power, time, and the degree of precision desired in the predicted results. The results of the molecular dynamics simulations can be considered to comprise a series of snapshots, taken as the dynamics simulation progresses. While in many cases it may be desirable to allow the simulations to proceed until they reach equilibrium (e.g., the point at which additional processing time no longer produces significant changes in the calculated optimized structure), that need not always be the case. Accordingly, the particular duration of the molecular dynamics optimization steps is not critical to the methods disclosed herein. In some preferred implementations, simulations are run for times in the range from about 100 ps to about 1 ns, and the structures produced by the method can include any individual snapshots taken within these time constraints.

Following the final optimization, the method outputs a final predicted 3-D structure of the entire protein and surrounding lipids, which may be generated in a standard format such as the protein data bank (pdb) format. As discussed above, the characterization of a particular predicted structure as a “final” structure will depend on the user's determination of the appropriate duration of the optimization step. Depending on which individual snapshot is chosen as the “final” structure, the particular predicted atomic coordinates may differ, as a result of additional optimization.

Accordingly, those skilled in the art will recognize that the output structures generated may differ even for a given set of input parameters. In particular embodiments, these differences can be expected to include differences in root mean square deviation of the predicted coordinates for atoms in the protein's amide backbone of less than or equal to about 2.0, or 3.0.

The techniques and apparatus described herein may have useful application in the modeling of any transmembrane protein having one or more membrane-spanning α-helices, where the protein's primary structure (amino acid sequence) is known, and, better yet, for which an experimental or theoretical helical template is available. In particularly advantageous implementations, the techniques and apparatus are useful in modeling the structure of having a relatively large number (e.g., about 4 or more) membrane-spanning α-helical regions, such as the seven-helical GPCRs.

The output structures can be used in further studies, including, for example, ligand docking studies using the HierDock or other similar protocols. These docking studies can be directed to modeling receptor binding sites, or to the purpose of identifying natural or synthetic receptor ligands, or to develop synthetic receptors that exhibit behavior analogous to naturally-occurring GPCRs. The predicted structures can also be useful in identifying regions of cellular receptors that bind microbial pathogens.

Subsequently using this model of the first step of pathogenesis, one could design competitive inhibitors either on the receptor or for the microbial surface structures.

The instant invention can be implemented in digital electronic circuitry, or in computer hardware, firmware, software, or combinations thereof. Apparatus of the invention can be implemented in one or more computer program products tangibly embodied in a machine-readable storage device for execution by a programmable processor; and method steps of the invention can be performed by a programmable processor executing a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

II. Definition

The terms used in this specification (including the ones listed below) generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. Thus such discussion should not be construed to be limiting. The scope an meaning of any use of a term will be apparent from the specific context in which the term is used.

“About” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

“Protein backbone structure” or grammatical equivalents herein is meant the three dimensional coordinates that define the three dimensional structure of a particular protein. The structures which comprise a protein backbone structure (of a naturally occurring protein) are the nitrogen, the carbonyl carbon, the α-carbon, and the carbonyl oxygen, along with the direction of the vector from the a-carbon to the β-carbon.

The protein backbone structure which is input into the computer can either include the coordinates for both the backbone and the amino acid side chains, or just the backbone, i.e. with the coordinates for the amino acid side chains removed. If the former is done, the side chain atoms of each amino acid of the protein structure may be “stripped” or removed from the structure of a protein, as is known in the art, leaving only the coordinates for the “backbone” atoms (the nitrogen, carbonyl carbon and oxygen, and the α-carbon, and the hydrogens attached to the nitrogen and α-carbon).

Optionally, the protein backbone structure may be altered prior to the analysis outlined below. In this embodiment, the representation of the starting protein backbone structure is reduced to a description of the spatial arrangement of its secondary structural elements. The relative positions of the secondary structural elements are defined by a set of parameters called supersecondary structure parameters. These parameters are assigned values that can be systematically or randomly varied to alter the arrangement of the secondary structure elements to introduce explicit backbone flexibility. The atomic coordinates of the backbone are then changed to reflect the altered supersecondary structural parameters, and these new coordinates are input into the system for use in the subsequent protein design automation. For details, see U.S. Pat. No. 6,269,312, the entire content incorporated herein by reference.

“Conformational energy” refers generally to the energy associated with a particular “conformation”, or three-dimensional structure, of a macromolecule, such as the energy associated with the conformation of a particular protein. Interactions that tend to stabilize a protein have energies that are represented as negative energy values, whereas interactions that destabilize a protein have positive energy values. Thus, the conformational energy for any stable protein is quantitatively represented by a negative conformational energy value. Generally, the conformational energy for a particular protein will be related to that protein's stability. In particular, molecules that have a lower (i.e., more negative) conformational energy are typically more stable, e.g., at higher temperatures (i.e., they have greater “thermal stability”). Accordingly, the conformational energy of a protein may also be referred to as the “stabilization energy.”

Typically, the conformational energy is calculated using an energy “force-field” that calculates or estimates the energy contribution from various interactions which depend upon the conformation of a molecule. The force-field is comprised of terms that include the conformational energy of the alpha-carbon backbone, side chain-backbone interactions, and side chain-side chain interactions. Typically, interactions with the backbone or side chain include terms for bond rotation, bond torsion, and bond length. The backbone-side chain and side chain-side chain interactions include van der Waals interactions, hydrogen-bonding, electrostatics and solvation terms. Electrostatic interactions may include Coulombic interactions, dipole interactions and quadrapole interactions). Other similar terms may also be included. Force-fields that may be used to determine the conformational energy for a polymer are well known in the art and include the CHARMM (see, Brooks et al., J. Comp. Chem. 4:187-217, 1983; MacKerell et al., in The Encyclopedia of Computational Chemistry, Vol. 1:271-277, John Wiley & Sons, Chichester, 1998), AMBER (see, Cornell et al., J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765) and DREIDING (Mayo et al., J. Phys. Chem. 1990, 94:8897) force-fields, to name but a few.

In a preferred implementation, the hydrogen bonding and electrostatics terms are as described in Dahiyat & Mayo, Science 1997 278:82). The force field can also be described to include atomic conformational terms (bond angles, bond lengths, torsions), as in other references. See e.g., Nielsen J E, Andersen K V, Honig B, Hooft R W W, Klebe G, Vriend G, & Wade R C, “Improving macromolecular electrostatics calculations,” Protein Engineering, 12: 657662(1999); Stikoff D, Lockhart D J, Sharp K A & Honig B, “Calculation of electrostatic effects at the amino-terminus of an alpha-helix,” Biophys. J., 67: 2251-2260 (1994); Hendscb Z S, Tidor B, “Do salt bridges stabilize proteins—a continuum electrostatic analysis,” Protein Science, 3: 211-226 (1994); Schneider J P, Lear J D, DeGrado W F, “A designed buried salt bridge in a heterodimeric coil,” J. Am. Chem. Soc., 119: 5742-5743 (1997); Sidelar C V, Hendsch Z S, Tidor B, “Effects of salt bridges on protein structure and design,” Protein Science, 7: 1898-1914 (1998). Solvation terms could also be included. See e.g., Jackson S E, Moracci M, elMastry N, Johnson C M, Fersht A R, “Effect of Cavity-Creating Mutations in the Hydrophobic Core of Chymotrypsin Inhibitor 2,” Biochemistry, 32: 11259-11269 (1993); Eisenberg, D & McLachlan A D, “Solvation Energy in Protein Folding and Binding,” Nature, 319: 199-203 (1986); Street A G & Mayo S L, “Pair-wise Calculation of Protein Solvent-Accessible Surface Areas,” Folding & Design, 3: 253-258 (1998); Eisenberg D & Wesson L, “Atomic solvation parameters applied to molecular dynamics of proteins in solution,” Protein Science, 1: 227-235 (1992); Gordon & Mayo, supra.

“Coupled residues” are residues in a molecule that interact, through any mechanism. The interaction between the two residues is therefore referred to as a “coupling interaction.” Coupled residues generally contribute to polymer fitness through the coupling interaction. Typically, the coupling interaction is a physical or chemical interaction, such as an electrostatic interaction, a van der Waals interaction, a hydrogen bonding interaction, or a combination thereof. As a result of the coupling interaction, changing the identity of either residue will affect the “fitness” of the molecule, particularly if the change disrupts the coupling interaction between the two residues. Coupling interaction may also be described by a distance parameter between residues in a molecule. If the residues are within a certain cutoff distance, they are considered interacting.

“Fitness” is used to denote the level or degree to which a particular property or a particular combination of properties for a molecule, e.g., a protein, are optimized. In certain embodiments of the invention, the fitness of a protein is preferably determined by properties which a user wishes to improve. Thus, for example, the fitness of a protein may refer to the protein's thermal stability, catalytic activity, binding affinity, solubility (e.g., in aqueous or organic solvent), and the like. Other examples of fitness properties include enantioselectivity, activity towards non-natural substrates, and alternative catalytic mechanisms. Coupling interactions can be modeled as a way of evaluating or predicting fitness (stability). Fitness can be determined or evaluated experimentally or theoretically, e.g. computationally.

Preferably, the fitness is quantitated so that each molecule, e.g., each amino acid will have a particular “fitness value”. For example, the fitness of a protein may be the rate at which the protein catalyzes a particular chemical reaction, or the protein's binding affinity for a ligand. In a particularly preferred embodiment, the fitness of a protein refers to the conformational energy of the polymer and is calculated, e.g., using any method known in the art. See, e.g. Brooks B. R., Bruccoleri R E, Olafson, B D, States D J, Swaminathan S & Karplus M, “CHARMM: A Program for Macromolecular Energy, Minimization, and Dynamics Calculations,” J. Comp. Chem., 4: 187-217 (1983); Mayo S L, Olafson B D & Goddard W A G, “DREIDING: A Generic Force Field for Molecular Simulations,” J. Phys. Chem., 94: 8897-8909 (1990); Pabo C 0 & Suchanek E G, “Computer-Aided Model-Building Strategies for Protein Design,” Biochemistry, 25: 5987-5991 (1986), Lazar G A, Desjarlais J R & Handel T M, “De Novo Design of the Hydrophobic Core of Ubiquitin,” Protein Science, 6: 1167-1178 (1997); Lee C & Levitt M, “Accurate Prediction of the Stability and Activity Effects of Site Directed Mutagenesis on a Protein Core,” Nature, 352: 448-451 (1991); Colombo G & Merz K M, “Stability and Activity of Mesophilic Subtilisin E and Its Thermophilic Homolog: Insights from Molecular Dynamics Simulations,” J. Am. Chem. Soc., 121: 6895-6903 (1999); Weiner S J, Kollman P A, Case D A, Singh U C, Ghio C, Alagona G, Profeta S J, Weiner P, “A new force field for molecular mechanical simulation of nucleic acids and proteins,” J. Am. Chem. Soc., 106: 765-784 (1984). Generally, the fitness of a protein is quantitated so that the fitness value increases as the property or combination of properties is optimized. For example, in embodiments where the thermal stability of a protein is to be optimized (conformational energy is preferably decreased), the fitness value may be the negative conformational energy; i.e., F=−E.

The “fitness contribution” of a protein residue refers to the level or extent f(iₐ) to which the residue iₐ, having an identity a, contributes to the total fitness of the protein. Thus, for example, if changing or mutating a particular amino acid residue will greatly decrease the protein's fitness, that residue is said to have a high fitness contribution to the polymer. By contrast, typically some residues iₐ in a protein may have a variety of possible identities a without affecting the protein's fitness. Such residues, therefore have a low contribution to the protein fitness.

“Dead-end elimination” (DEE) is a deterministic search algorithm that seeks to systematically eliminate bad rotamers and combinations of rotamers until a single solution remains. For example, amino acid residues can be modeled as rotamers that interact with a fixed backbone. The theoretical basis for DEE provides that, if the DEE search converges, the solution is the global minimum energy conformation (GMEC) with no uncertainty (Desmet et al., 1992).

Dead end elimination is based on the following concept. Consider two rotamers, iᵣ and iₜ, at residue i, and the set of all other rotamer configurations {S} at all residues excluding i (of which rotamer jₛ is a member). If the pair-wise energy contributed between iᵣ and jₛ is higher than the pair-wise energy between iₜ and jₛ for all {S }, then rotamer iᵣ cannot exist in the global minimum energy conformation, and can be eliminated. This notion is expressed mathematically by the inequality.

$$E ( {i}_{r} ) + {\sum }_{j \neq i}^{N} E ( {i}_{r} , {j}_{s} ) > E ( {i}_{t} ) + {\sum }_{j \neq i}^{N} E ( {i}_{t} , {j}_{s} ) \{ S \} \qquad ( Equation A )$$

If this expression is true, the single rotamer iᵣ can be eliminated (Desmet et al., 1992).

In this form, Equation A is not computationally tractable because, to make an elimination, it is required that the entire sequence (rotamer) space be enumerated. To simplify the problem, bounds implied by Equation A can be utilized:

$$E ( {i}_{r} ) + {\sum }_{j \neq i}^{N} min ( s ) E ( {i}_{r} , {j}_{s} ) > \\ E ( {i}_{t} ) + {\sum }_{j \neq i}^{N} max ( s ) E ( {i}_{t} , {j}_{s} ) \{ S \} \qquad ( Equation B )$$

Using an analogous argument, Equation B can be extended to the elimination of pairs of rotamers inconsistent with the GMEC. This is done by determining that a pair of rotamers iᵣ at residue i and jₛ at residue j, always contribute higher energies than rotamers iᵤ and jᵥ with all possible rotamer combinations {L}. Similar to Equation B, the strict bound of this statement is given by:

$$ɛ ( {i}_{r} , {j}_{s} ) + {\sum }_{k \neq i , j}^{N} min ( t ) ɛ ( {i}_{r} , {j}_{s} , {k}_{t} ) > \\ ɛ ( {i}_{u} , {j}_{v} ) + {\sum }_{k \neq i , j}^{N} max ( t ) ɛ ( {i}_{u} , {j}_{v} , {k}_{i} ) \qquad ( Equation C )$$

where ε is the combined energies for rotamer pairs
ε(iᵣ,jₛ)=E(iᵣ)+E(jₛ)+E(iᵣ,jₛ (Equation D),
and
ε(iᵣ,jₛ,kₜ)=E(iᵣ,kₜ)+E(jₛ,kₜ (Equation E).

This leads to the doubles elimination of the pair of rotamers iᵣ and jₛ, but does not eliminate the individual rotamers completely as either could exist independently in the GMEC. The doubles elimination step reduces the number of possible pairs (reduces S) that need to be evaluated in the right-hand side of Equation 6, allowing more rotamers to be individually eliminated.

The singles and doubles criteria presented by Desmet et al. fail to discover special conditions that lead to the determination of more dead-ending rotamers For instance, it is possible that the energy contribution of rotamer it is always lower than iᵣ without the maximum of it being below the minimum of iᵣ. To address this problem, Goldstein 1994 presented a modification of the criteria that determines if the energy profiles of two rotamers cross. If they do not, the higher energy rotamer can be determined to be dead-ending. The doubles calculation significantly more computational time than the singles calculation. To accelerate the process, other computational methods have been developed to predict the doubles calculations that will be the most productive (Gordon & Mayo, 1998). These kinds of modifications, collectively referred to as fast doubles, significantly improved the speed and effectiveness of DEE.

Several other modifications also enhance DEE. Rotamers from multiple residues can be combined into so-called super-rotamers to prompt further eliminations (Desmet et al., 1994; Goldstein, 1994). This has the advantage of eliminating multiple rotamers in a single step.. In addition, it has been shown that “splitting” the conformational space between rotamers improves the efficiency of DEE (Pierce et al., 2000). Splitting handles the following special case. Consider rotamer iᵣ. If a rotamer iₜ₁ contributes a lower energy than iᵣ for a portion of the conformational space, and a rotamer iₜ₂ has a lower energy than iᵣ for the remaining fraction, then iᵣ can be eliminated. This case would not be detected by the less sensitive Desmet or Goldstein criteria. In the preferred implementations of the invention as described herein, all of the described enhancements to DEE were used.

For further discussion of these methods see, Goldstein, R. F. (1994), Efficient rotamer elimination applied to protein side-chains and related spin glasses, Biophysical Journal 66, 1335-1340; Desmet, J., De Maeyer, M., Hazes, B. & Lasters, I. (1992), The dead-end elimination theorem and its use in protein side-chain positioning. Nature 356,539-542; Desmet, J., De Maeyer, M. & Lasters, I. (1994), In The Protein Folding Problem and Tertiary Structure Prediction (Jr., K. M. & Grand, S. L., eds.), pp. 307-337 (Birkhauser, Boston); De Maeyer, M., Desmet, J. & Lasters, I. (1997), All in one: a highly detailed rotamer library improves both accuracy and speed in the modeling of side chains by dead-end elimination, Folding & Design 2, 53-66, Gordon, D. B. & Mayo, S. L. (1998), Radical performance enhancements for combinatorial optimization algorithms based on the dead-end elimination theorem, Journal of Computational Chemistry 19, 1505-1514; Pierce, N. A., Spriet, J. A., Desmet, J., Mayo, S. L., (2000), Conformational splitting: A more powerful criterion for dead-end elimination; Journal of Computational Chemistry 21, 999-1009.

The methods of the invention may include steps of comparing sequences to each other, including wild-type sequence to one or more mutants. Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, “-”, or “Δ”) in the polymer sequence not containing the inserted or deleted residue.

“Homologous”, in all its grammatical forms and spelling variations, refers to the relationship between two proteins that possess a “common evolutionary origin”, including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, refers to the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

“Polypeptide,” “peptide” or “protein” are used interchangeably to describe a chain of amino acids that are linked together by chemical bonds called “peptide bonds.” A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

“Rotamer” is defined as a set of possible conformers for each amino acid or analog side chain. See Ponder, et al., Acad. Press Inc. (London) Ltd. pp. 775-791 (1987); Dunbrack, et al., Struc. Biol. 1(5):334-340 (1994); Desmet, et al., Nature 356:539-542 (1992). A “rotamer library” is a collection of a set of possible/allowable rotameric conformations for a given set of amino acids or analogs. There are two general types of rotamer libraries: “backbone dependent” and “backbone independent.” A backbone dependent rotamer library allows different rotamers depending on the position of the residue in the backbone; thus for example, certain leucine rotamers are allowed if the position is within an α helix, and different leucine rotamers are allowed if the position is not in an α-helix. A backbone independent rotamer library utilizes all rotamers of an amino acid at every position. In general, a backbone independent library is preferred in the consideration of core residues, since flexibility in the core is important. However, backbone independent libraries are computationally more expensive, and thus for surface and boundary positions, a backbone dependent library is preferred. However, either type of library can be used at any position.

“Raw alignment score” or the score of an alignment, S, is calculated as the sum of substitution and gap scores. Substitution scores are given by a look-up table (see PAM, BLOSUM62 etc.). Gap scores are typically calculated as the sum of G, the gap opening penalty and L, the gap extension penalty. For a gap of length n, the gap cost would be G+Ln. The choice of gap costs, G and L is empirical and may be defined by user, but it is customary to choose a high value for G (e.g. 10-15) and a low value for L (e.g. 1-2). BLAST 2.0 and PSI-BLAST use “affine gap costs” which charge the score -a for the existence of a gap, and the score -b for each residue in the gap. A gap of k residues therefore receives a total score of -(a+bk) and a gap of length 1 receives the score -(a+b). Gap creation and extension variables a and b are inherent to the scoring system in use.

“Bit score” is a numeric value (positive integer) calculated by the BLAST program based on the goodness of match between the query sequence and the hit sequence. In general, the higher the sequence homology/identity, and the longer the homologous region, the higher the bit score. The value S′ is derived from the raw alignment score S in which the statistical properties of the scoring system used have been taken into account. Because bit scores have been normalized with respect to the scoring system, they can be used to compare alignment scores from different searches.

To convert a raw score S into a normalized score S′ expressed in bits, one uses the formula S′=(lambda*S−ln K)/(ln 2), where lambda and K are parameters dependent upon the scoring system (substitution matrix and gap costs) employed (Karlin & Altschul (1990) Methods for assessing the statistical significance of molecular sequence features by using general scoring schemes. Proc. Natl. Acad. Sci. USA 87:2264-2268; Altschul & Gish (1996) Local alignment statistics. Meth. Enzymol. 266:460-480; Altschul et al (1997) Gapped BLAST and PSI-BLAST: a new generation of protein database search programs. Nucleic Acids Res. 25:3389-3402. All incorporated herein by reference). For determining S′, the more important of these parameters is lambda. The “lambda ratio” quoted here is the ratio of the lambda for the given scoring system to that for one using the same substitution scores, but with infinite gap costs (Altschul & Gish, supra). This ratio indicates what proportion of information in an ungapped alignment must be sacrificed in the hope of improving its score through extension using gaps. It is empirically found that the most effective gap costs tend to be those with lambda ratios in the range 0.8 to 0.9.

“E-value” or “expectation value” represents the number of different alignments with scores equivalent to or better than S that are expected to occur in a database search by chance. The lower (closer to 0) the E value, the more significant the score. An identical hit sequence has the lowest possible E-value, or 0. E-value is generally expressed as e⁻ⁿ, where n is an integer.

III. Exemplary Embodiments of the Invention

(A) Force Fields

DREIDING force field

In a preferred embodiment, such as in the examples below, all calculations for the proteins used the DREIDING force field (FF) (Mayo et al., 1990) with charges from CHARMM22 (MacKerell et al., 1998) unless specified otherwise. The nonbond interactions were calculated using the cell multipole method (Ding et al., 1992) in MPSim (Lim et al., 1997).

The ligands were described with the DREIDING FF (Mayo et al., 1990) using charges from quantum mechanics calculations on the isolated ligand; electrostatic potential charges calculated using Jaguar, Ver. 4.0 (Schrodinger, Portland, Oreg.). For the lipids, the DREIDING FF with QEq charges were used (Rappé and Goddard, 1991). Some calculations were done in the vacuum (e.g., final optimization of receptor structure to approximate the low dielectric membrane environment). For structural optimization in the solvent (water), the analytical volume Generalized-Born (AVGB, see Zamanakos, 2002) approximation to Poisson-Boltzmann (PB) continuum solvation were used.

The DREIDING FF were used due to its generic applicability to all molecules constructed from main group elements (particularly all organics), inasmuch as the methods are used to predict the binding site and energy for a diverse set of ligands of interest to pharmacology. Indeed, it was found that the minimized structure for bovine rhodopsin deviates from the crystal structure by only 0.29 Å coordinate root mean-square error (CRMS). The DREIDING FF with CHARMM22 charges has been validated for molecular dynamics simulations and binding energy calculations for many proteins (Brameld and Goddard, 1999; Datta et al., 2003, 2002; Wang et al., 2002; Kekenes-Huskey et al., 2003; Floriano et al., 2004b) with similar accuracy.

Alternative Force Fileds

Although the DREIDING Force Field (see Mayo et al., J. Phys. Chem. 1990, 94:8897) is chosen an illustrative force filed for use in this procedure, other force fields, such as AMBER (see, Cornell et al., J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765); AMBER/OPLS (Damm et al., J. Comp. Chem. 18: 1955-1970, 1997; Jorgensen et al., J. Am. Chem. Soc. 118: 11225-11236, 1996; Jorgensen & Tirado-Rives, J., J. Am. Chem. Soc. 110: 1657-1666, 1998; Kaminski et al., J. Phys. Chem. 98: 13077-13082, 1994); CHARMM (Brooks et al. J Comput Chem 4:187-217, 1983; Feller et al., Biophys. J. 73, 2269-2279, 1997; MacKerell et al., J. Phys. Chem., B 102: 3586-3617, 1998; Mackerell et al., J. Amer. Chem. Soc. 117: 11946-11975, 1995; Momany & Rone J. Comp. Chem. 13: 888-900, 1992; Pavelites et al., J. Comp. Chem. 18, 221-239, 1997; Schlenkrich et al. Empirical Potential Energy Function for Phospholipids: Criteria for Parameter Optimization and Applications in “Biological Membranes: A Molecular Perspective from Computation and Experiment,” K. M. Merz and B. Roux, Eds. Birkhauser, Boston, pp 31-81, 1996); Discover (cvff-Dauber-Osguthorpe, P. et al., Proteins: Structure, Function and Genetics, 4, 31-47, 1988; cff-Hagler, A. T.; Ewig, C. S. “On the use of quantum energy surfaces in the derivation of molecular force fields”, Comp. Phys. Comm. 84, 131-155, 1994; Hwang, M. -J. et al., J. Amer. Chem. Soc. 116: 2515-2525, 1994; Maple, J. R. et al., J. Comput. Chem. 15: 162-182, 1994; Maple, J. R. et al., Israel J. Chem. 34: 195 -231, 1994); ECEPP/2 (Momany et al., J. Phys. Chem. 79: 2361-2381, 1975; Nemethy et al., J. PHys. Chem. 87: 1883-1887, 1983; Sippl et al., J. Phys. Chem. 88: 6231-6633, 1984); GROMOS (Hermans, J. et al., Biopolymers 23: 1, 1984; Ott and Meyer J Comp Chem 17: 1068-1084, 1996; van Gunsteren et al., “The GROMOS force field” in Encyclopaedia of Computational Chemistry, 1997); MM2 (Allinger J. Am. Chem. Soc. 99: 8127-8134, 1997; Allinger et al., J. Comp. Chem. 9: 591-595, 1988; Lii et al., J. Comp. Chem. 10: 503-513, 1989); MM3 (Allinger et al., J. Am. Chem. Soc. 111: 8551-8565, 1989; Hay et al., J. Molecular Structure 428: 203-219, 1998; Lii & Allinger J. Am. Chem. Soc. 111: 8566-8575, 1989; Lii & Allinger J. Am. Chem. Soc. 111: 8576-8582, 1989; Lii & Allinger J. Comp. Chem. 12: 186-199, 1991; Lii & Allinger J. Comp. Chem. 19: 1001-1016, 1998); MM4 (Allinger et al., J. Comp. Chem. 17: 642-668, 1996; Allinger et al., J. Comp. Chem. 17: 747-755, 1996; Allinger and Fan, J. Comp. Chem.18: 1827-1847, 1997; Nevens et al., J. Comp. Chem. 17: 669-694, 1996; Nevins et al., J. Comp. Chem. 17: 695-729, 1996; Nevins and Allinger, J. Comp. Chem. 17: 730-746, 1996); MMFF94 (Halgren J. Am. Chem. Soc. 114: 7827-7843, 1992; Halgren J. Comp. Chem. 17, 490-519; 1996; Halgren J. Comp. Chem. 17: 520-552, 1996; Halgren J. Comp. Chem. 17: 553-586, 1996; Halgren and Nachbar J. Comp. Chem. 17: 587-615, 1996; Halgren J. Comp. Chem. 17: 616-641, 1996); Tripos (Clark et al., J. Comp. Chem. 10: 982-1012, 1989); Comparisons and Evaluations (Engler et al., J. Am. Chem. Soc. 95: 8005-8025, 1973; Gundertofte et al., J. Comp. Chem. 12: 200-208, 1991; Gundertofte et al., J. Comp. Chem. 17: 429-449, 1996; Hall and Pavitt, J. Comp. Chem. 5: 441-450, 1984; Hobza et al., J. Comp. Chem. 18: 1136-1150, 1997; Kini et al., J. Biomol. Structure and Dynamics 10: 265-279, 1992; Roterman et al., J. Biomol. Struct. and Dynamics 7: 391-419, 1989; Roterman et al., J. Biomol. Struct. and Dynamics 7: 421-452, 1989; Whitlow and Teeter, J. Am. Chem. Soc. 108: 7163-7172, 1989); AMBER94; MMFF; MMFFs; OPLS (Kaminski et al., J. Phys. Chem. B 105: 6474-6487, 2001); OPLS-AA (Jorgensen et al., JACS 118: 11225, 1996); UFF (Rappe et al., J. Am. Chem. Soc. 114: 10024, 1992; Casewit et al., J. Am. Chem. Soc. 114: 10035, 1992; Casewit et al., J. Am. Chem. Soc. 114: 10046, 1992; Rappe et al., Inorg. Chem. 32: 3438, 1993), etc., can also be used to calculate binding energies.

In addition, the functional forms of the non-bond energy in calculation can have different forms. The dielectric constant in the Coulomb term can be distance dependent. The charges for both the protein and the ligand can be varied. This includes charges from experiment, or charges based on various models, such as, but not limited to, QEq (“Charge Equilibration,” see Rappe and Goddard, J. Phys. Chem. 95: 3358-63, 1991), Del Re (Del Re J. Chem. Soc. 4031, 1958; Poland and Scheraga Biochemistry 6: 3791, 1967; Coefficients modified in MMP to take into account pi contributions—values can be modified by the user), Gasteiger (or PEOE, see Gasteiger and Marsili, Tetrahedron 36: 3219-28, 1980), MPEOE (DQP) (No et al., J. Phys. Chem. 94: 4732, 1990; No et al., J. Phys. Chem. 94: 4740, 1990; Park et al., J. Comp. Chem. 14:1482, 1993), ReaxFF (Goddard et al., “The ReaxFF Polarizable Reactive Force Fields for Molecular Dynamics Simulation of Ferroelectrics,” Presented at Fundamental Physics of Ferroelectrics 2002, held in Washington D.C., Feb. 3-6, 2000, Chairmen: Ron Cohen and Takeshi Egami), etc. The van der Waals term can have different forms, such as a Morse potential (U(x)=Dₑ[1−e^{-(x-r)}ₑ]²) instead of a Leonard-Jones potential. The 6-12 Leonard-Jones potential can be made 6- 10, or even softer to allow closer contact. Finally the hydrogen bond term can have several different variations. The three body form may be used in calculation, but two-body or four body form is common too, and can also be similarly used. Further, in some force fields, hydrogen bond can also be treated implicitly as part of the Coulomb term.

Alternative Solvation Methods

Solvation is an important factor in determining biomolecular stability and binding properties. In certain embodiments, implicit solvent can be used to minimize the structures and to calculate binding energies. These implicit solvent model includes, but not limited to Surface Generalized Born (SGB) model (Ghosh et al. J. Phys. Chem. 102: 10983-10990, 1998), Solvent Accessible Surface Area (SASA)/Analytical Volume Generalized Born (AVGB) model (Zamanakos, Ph.D. Thesis, California Institute of Technology, Pasadena, Calif., 2001), and Poisson-Boltzmann (PB) model.

In addition, explicit solvent molecules can be easily added to the calculation of binding energy, as long as the solvation model is accurate enough to account for the solvation effect of ligand binding to the target protein. This can usually be used in the evaluation of the best cases for final selection in the design.

Many commercial software packages support calculations for various explicit and/or implicit solvation models, such as Impact, a molecular mechanics program specifically designed to handle large macromolecular simulations. The program effortlessly treats simulations on ligand-protein systems in solvation or in vacuo, enabling the study of large systems in a timely manner. For example, Impact's explicit solvation offers the possibility of setting up calculations in solvent boxes, where water or user-defined solvent molecules are placed at distinct sites of a solvent box. Periodic boundary conditions are also available. Continuum models greatly enhance computational speed. Impact's continuum solvation method is a surface area version of the generalized Born model (SGB). The SGB model has been shown to give significant improvement in accuracy over the uncorrected GB model. The large number of solvent molecules used in solution-phase simulations is also avoided using the PBF (Poisson Boltzmann solver) solvation model in Impact (Cortis and Friesner J. Comp. Chem. 18: 1570, 1997; Cortis and Friesner J. Comp. Chem. 18: 1591, 1997). This model offers additional savings in computational expense using a customizable finite element mesh.

Another exemplary software package that employs multiple force fields and matching solvation methods is the MacroModel® program from Schrodinger (Portland, Oreg.).

Alternative Binding Energy Calculation

It is always important to get the correct relative binding energies for different ligands. As an alternative method, minimization in the Potential of Mean Force (PMF) from implicit continuum solvent model can also be used to calculate binding (free) energies. As another alternative, the average dynamic free energy instead of free energy form a single conformation can be used. In addition, other methods may (at least in theory) be used to calculate binding free energies, although they may require significantly longer computation time. These methods include Free Energy Perturbation (FEP) (Becker et al., Marcel Dekker, New York, 2001), and methods based on thermodynamic cycles. See the AMBER package described in the section above. Obviously, such methods can be more realistically implemented with a faster and more powerful computer.

Protein Side-chain Modeling

There are several side chain modeling methods that can be incorporated into the methods of the invention. These side chain modeling methods include SCWRL, SCAP, and methods based on branch-and-bound, dead-end-elimination algorithms.

SCRWL (Sidechain placement With a Rotamer Library):

SCWRL (Bower et al., J. Mol. Biol. 267: 1268-82, 1997) is a program for adding side-chains to a protein backbone based on the backbone-dependent rotamer library. The library provides lists of chi1-chi2-chi3-chi4 values and their relative probabilities for residues at given phi-psi values, and explores these conformations to minimize sidechain-backbone clashes and sidechain-sidechain clashes. It is possible to get output from the program at any of the three steps: best library rotamers, no clashes relieved; backbone clashes relieved; backbone and sidechain-sidechain clashes relieved.

One recent version of the program (scwr12.7) achieves a prediction rate for chi1 dihedral angles correct within 40 degrees for all residues of 81.0%.

The method used by SCWRL is based on the hypothesis that a great deal of the information needed for sidechain positioning is contained in the local mainchain conformation of each residue, but that a search strategy to resolve steric exclusions is also necessary for the most accurate predictions. SCWRL is a single self-contained program optimized for speed and accuracy. SCWRL is designed to take full advantage of the rotamer approximation and the strong backbone dependencies rotamers display to create an initial placement for each residue, followed by systematic searches to resolve steric clashes.

It begins with the mainchain atoms N, C_{α}, C, and O from a protein structure. The mainchain dihedral angles phi (Φ) and psi (Ψ) calculated by SCWRL, together with the sequence, are used to look up an ordered list of rotamers for each residue from a rotamer database. Each of these potential rotamers is built, using bond lengths and angles from the AMBER 4.1 parameter set, and the set of rotamers is searched for the minimum steric clash to create the output structure.

The vdw energy function in SCWRL is a simple repulsive steric clash check. For a pair of atoms, the energy of interaction is given by:
E=0.0 R>R0
E=(R/R0)(−57.273)+57.273R0>=R>=0.83R0 Eq. A)
E=10.0 R<0.83R0

where R is the distance between the atoms and R0 is the sum of their radii. The linear portion of the function approximates the repulsive curve of a Leonard-Jones potential. A graph showing the energy function used by SCWRL and a Leonard-Jones potential (light line) is displayed in the SCWRL website.

To make the steric term more forgiving on the rigid rotamers, the radii of atoms are reduced roughly 15% from their van der Waals radii, to values which approximate the distance where a Leonard-Jones potential would become repulsive.

Radii that can be used for steric clash checks are listed below:

Atom type

| | C | N | O | S |
|---|---|---|---|---|
| Radius(Angstroms) | 1.6 | 1.3 | 1.3 | 1.5 |

Note: radii increased to these values, SCWRL2.0 (R. Dunbrack, Aug. 21, 1997).

Each rotamer is also given a local rotamer energy based on the probability of that rotamer in the backbone-dependent rotamer library. These energies are taken from the probabilities of the backbone-dependent rotamer library, as −ln(pi/p0), where p0 is the probability of the most probably rotamer, and pi is the probability of the i-th rotamer (assuming kT=1).

SCWRL first calculates possible disulfide bonds from rotamers of cysteine residues and resulting distances between sulfur atoms. These residues are then frozen in their disulfide conformation, and the sidechain atoms are treated as part of the backbone scaffold in determining the conformations of the other residue types.

To overcome the combinatorial nature of the sidechain packing problem, the search strategy does not involve a search of every rotamer of every sidechain, but rather takes a structure with residues in their most favorable backbone-dependent rotamers and systematically resolves the conflicts that arise from that structure. Each residue begins in its most favored rotamer, according to the rotamer database used. This is the first stage structure. When a sidechain from the first stage structure has a steric clash as defined by Eq. A with the (fixed) mainchain, the rotamer for that residue is changed to progressively less favorable rotamers until one is found that does not conflict with the mainchain. The second stage structure has all of these sidechain to mainchain clashes relieved.

At this point, there will be some sidechain to sidechain clashes. Where these occur, the two clashing residues are put in a “cluster” of residues which interact. The clashing residues are allowed to explore all of their respective rotamers, save those which cause sidechain to mainchain clashes, and any residues which clash with these “rotating” residues are added to the cluster and themselves allowed to explore all their rotamers. In this way, clusters of residues grow by clashing with residues not in the original cluster. Clusters can also merge when two rotamers from residues in different clusters clash, in which case all of the residues from the two clusters become part of the same cluster. Sidechains which do not clash with the mainchain, and are never involved in a steric clash with an activated residue, are left in their most favorable backbone-dependent rotamer.

When the clusters have grown to their final size, each one represents an exclusive subset of residues which are allowed to interact with each other. Each cluster is solved, in turn, through a combinatorial search to find its minimum steric clash score. When all of the clusters have been solved, the stage three structure is output as the solution.

The search procedure tests each residue and combination of residues in the order they were added to the cluster. Rotamers for each residue are tested in order of decreasing favorability. The first combination of rotamers which is found to have a steric clash score of zero is taken as the final solution. If no such combination is found, all the rotamer combinations are searched, and the combination with the minimum steric clash score is taken as the final solution.

Occasionally, clusters grow too large to be solved quickly with a combinatorial search. When such a large number of combinations is reached, the cluster is broken into sub-clusters to speed the solution time. In some cases, the limit is set for clusters that cannot be solved by the combinatorial search in approximately one second, which is reached for clusters containing more than 1.5E7 rotamer combinations, about 15 residues. A large cluster is parsed by finding the residue in that cluster whose removal from the cluster results in the smallest sub-clusters. Then each of the sub-clusters is solved in the presence of each of the “keystone” residue's potential rotamers. For example, in a 2 1-residue cluster where every residue has three potential rotamers, the combinations to search will number 3²¹, or 1.0 E10. If a residue in this cluster is found which divides the cluster into two 10-residue sub-clusters, then the combinations to search will number 3(3¹⁰+3¹⁰)=3.5E5.

For the purposes of searching alternate conformations to resolve a cluster of clashing sidechains, residues are searched in order from high entropy sidechains to low entropy sidechains. E.g. a sidechain with p0=0.1 is searched before a sidechain with p0=0.9. Since SCWRL favors early solutions to the cluster over later ones, if the energies are equal, this is a small benefit. As before, all residues which clash in their library conformations are searched (in order of decreasing entropy) before sidechains which clash with lower probability rotamers of the original clustering residues.

This parsing of clusters into sub-clusters is a recursive process, and if the sub-clusters still contain more combinations than the cutoff, or if a keystone residue fails to break a cluster into non-interacting sub-clusters, the remaining clusters are passed down to a new level for additional parsing. Only in some very rare cases, the parse routine cannot find a subset of keystones which breaks the cluster up fast enough to overcome the combinatorics.

For more details of the SCWRL program, including licensing information, see the SCWRL website maintained by R. Dunbrack (last modified in Sep. 30, 2003).

SCWRL3.0

The most recent version of SCWRL is SCRWL3.0 (Canutescu, Shelenkov, and Dunbrack, Jr. A graph theory algorithm for protein side-chain prediction. Protein Science 12, 2001-2014, 2003). SCWRL3.0 is a completely new version of the SCWRL program for prediction of protein side-chain conformations. SCWRL3.0 is based on a new algorithm based on graph theory that solves the combinatorial problem in side-chain prediction more rapidly than any other available program. SCWRL3.0 is more accurate than previous versions of SCWRL, while the new algorithm will allow for development of more sophisticated energy functions and for incorporation of side-chain flexibility around rotameric positions.

SCWRL3.0 is much faster than previous versions of SCWRL. Previous versions of SCWRL used two parameters to reduce the complexity of the search problem among clusters of interacting side chains. These are EBBMIN and EPAIRMIN. EBBMAX is the maximum backbone/sidechain interaction energy for a rotamer to be included in the calculations. EPAIRMIN is the minimum side-chain/side-chain interaction energy for two residues to be considered connected in determining clusters of interacting residues that must be resolved to find the global minimum energy. Raising EBBMAX increases the size of the calculation by increasing the number of rotamers for each side chain. Decreasing EPAIRMIN increases the size of the calculation by increasing the number of neighboring side chains interacting with each rotamer of a side chain. In addition, SCWRL3.0 is also more accurate than SCWRL2.95.

The related Backbone-dependent Rotamer Library website maintained by Dunbrack (http://dunbrack.fccc.edu/bbdep) is last updated in Aug. 29, 2003. As is well-known in the art, certain rotameric states will be higher in energy than others because of steric interactions that cause the sidechain to twist out of the way of neighboring atoms, inflicting a high dihedral energy on the residue. These interactions can be “backbone-independent”, that is, not depending on the conformation of the local backbone of the residue, or “backbone-dependent”, that is, depending on the local backbone conformation as determined by the backbone dihedrals phi and psi. The backbone-independent rotamer library is similar to the familiar Ponder & Richards rotamer library (J. Mol. Biol. 193: 775-791, 1987). The backbone-dependent rotamer library defines a rotamer distribution for small ranges of phi and psi, basically a rotamer library for every 10×10 degree box of phi and psi.

The site mentioned above includes backbone-independent and backbone-dependent rotamer libraries and the SCWRL program for making sidechain conformation predictions from the library and input phi and psi values and for evaluating the rotamers and chi angles of a preliminary x-ray, NMR, or model structure in comparison to the experimental distributions of rotamers and chi angles in the Protein Databank.

The library is subject to frequent update (every couple of months). The May 2002 version of the library contains 850 chains from the PDB of resolution 1.7 Å or better, and less than 40% homology with other chains in the set. The list was determined from the Dunbrack group's algorithm for finding sets of PDB chains with maximum sequence identities and resolution cutoffs. More specifically, the chains used in constructing the database are all from x-ray crystallographic structures from the Brookhaven Protein Databank (PDB). The algorithm for selecting these lists is similar to that of Hobohm and Sander. The only difference is the addition of resolution cutoffs, so that one gets the largest list possible with PDB entries of a certain resolution or better, and also that the algorithm favored higher resolution structures over lower resolution structures (by proceeding from high resolution to low resolution in the reject-until-done procedure). These lists are described on the related PISCES web site (http://www.fccc.edu/research/labs/dunbrack/pisces/).

SCAP (Xiang and Honig, J. Mol. Biol. 311: 421-430, 2001):

Scap is a program for protein side-chain prediction and residue mutation. The program can make prediction on all residues or on certain residues in a protein of multiple chains. It can automatically detect if the residue to be predicted or mutated is backbone only, or complete with all side-chain atoms. If the residue is backbone only, it will first add side-chains and then do predictions; if the residue is to be mutated, the residue is first mutated accordingly and prediction is then performed.

The scap performs side-chain prediction using coordinate rotamer libraries. There are four coordinate rotamer libraries included in the library: side_small_rotamer, side_medium_rotamer, side_large_rotamer, and side_mix_rotamer. Side_small_rotamer library has 214 side-chain rotamers with 40-degree chi angle cutoff. This is equivalent to the 214 sidechain rotamer library of Tuffery's with standard bond angle and length; side_medium_rotamer has 3222 side-chain rotamers compiled from 297 chains with 20 degree cutoff and 96% representation; side_large_rotamer has 6487 side-chain rotamers compiled from 297 chains with 10 degree cutoff and 96% representation; side-mix-rotamer is a mix of side_medium_rotamer and side_large_rotamer. It includes all rotamers from side_large_rotamer except for ARG, LYS and MET which come from side_medium_rotamer. The more coordinate rotamer and dihedral angle rotamer libraries can be downloaded from website: http://trantor.bioc.columbia.edu/˜xiang/sidechain/index.html. More detailed information about the methods and algorithms in scap can be found in the paper: Xiang Z, Honig B. Extending the accuracy limits of prediction for side-chain conformations. J. Mol. Biol. 2001 311:421-30 (incorporated herein by reference); and Xiang, Z; Honig, B. Colony energy improves prediction of exposed sidechain conformations. The software and manual can be downloaded at: http://trantor.bioc.columbia.edu/˜xiang/jackal/index.html.

Scap support the following platform: SGI, Sun Solarius, Linux and Microsoft Window.

(B) The 3-D structure prediction protocol (e.g. MembStruk3.5)

The subject 3-D structure prediction methods such as MembStruk use the hydrophobic profile of amino acid sequences, preferably multisequence alignment of amino acid sequences of the target protein (e.g. GPCRs) to assign the TM regions. This is combined with a series of steps of a Monte Carlo-like systematic search algorithm to optimize the rotation and translational orientation of the TM regions. This search algorithm allows the structure to get over large barriers between various favorable positions in the conformation space, and make the conformational search more comprehensive (i.e. not trapped in less-favorable and/or smaller conformation spaces between large barriers). This is followed by molecular dynamics (MD) calculations at a variety of coarse-grain to fine-grain levels in explicit lipid bilayer.

The subject method includes energy optimization to determine the rotation of helices in the seven-helical TM bundle. It also includes optimization of the helix translations along their axes and rotational optimization using hydrophobic moment of the helices. The MembStruk3.5 procedure for predicting structures of multipass membrane proteins (such as GPCRs) comprises the following steps:

1. Prediction of TM regions from analysis of the primary sequence (this step is preferably conducted using a protocol such as TM2ndS).

2. Assembly and coarse-grain optimization of the TM bundle.

3. Optimization of individual TM regions (e.g. helices).

4. Rigid-body dynamics of the TM bundle in a lipid bilayer.

5. Addition of interhelical loops and optimization of the full structure.

Some of the details of these steps are discussed below. We should emphasize here that various versions of MembStruk protocols are merely preferred embodiments of the 3D structure prediction protocol described herein, and should be in no way limiting with respect to the scope of the invention. Secondly, the steps described below can be all automated into a single procedure, preferably executed by a computer program. Thus in a preferred embodiment, the target sequence is fed to a MembStruk-like program, and the result at the end is a final three-dimensional structure for the protein in the lipid bilayer.

Step 1: Prediction of TM regions (e.g. TM2ndS)

In general, prediction of the TM regions for multipass membrane proteins from their primary amino acid sequences partly rests on the assumption that the outer regions of the TM regions, such as helices in contact with the hydrophobic tails of the lipids, should be hydrophobic, and that this character should be largest near the center of the membrane. The TM2ndS method uses this concept to generate a hydrophobic profile.

Step 1a: Obtaining Primary Amino acid Sequence and (Optional) Multi-Sequence Alignment

In certain embodiments, the methods of the invention can directly use the primary amino acid sequence (including amino acid sequence translated from a polynucleotide sequence) for conversion to hydrophobicity values.

In other embodiments, the methods of the invention take advantage of a multisequence alignment of the target primary amino acid sequence. Such multisequence alignment result is used to derive consensus hydrophobicity for every residue position in the alignment, which is equivalent to the hydrophobicity values of the original primary amino acid sequence.

In either embodiments, a hydrophobic profile can be established by converting amino acid sequence or sequence alignment information into hydrophobicity values using a hydrophobicity scale. Peak signal analysis can then be performed based on the hydrophobic profile plot.

In certain embodiments, the Prift hydrophobicity scale (Cornette et al., 1987, incorporated herein by reference in its entirety) may be used. But in certain cases, the hydrophobicity index value for Arg in the Prift hydrophobicity scale may be opposite that expected for a charged residue, thus leading to potentially incorrect assignments.

Thus in preferred embodiments of the invention, the Eisenberg hydrophobicity scale (Eisenberg et al., 1982, incorporated herein by reference in its entirety), which is based on sound thermodynamic arguments, may be used. This scale has a range from −1.76 to 0.73 and works well for Arg and other residues to give consistent TM predictions for the many systems so far investigated.

In those embodiments where multi-sequence alignment is necessary, the target sequence is used as a query to retrieve an ensemble of homologous sequences meeting certain pre-determined criteria. To retrieve homologous sequences, sequence search engines/programs such as NCBI's BLASTp search (Altschul et al., 1990, 1997, incorporated herein by reference in its entirety) may be used. Among the retrieved hit sequences, those with sequence identity of about 20-90%, and/or bit scores >200, but not identical (to avoid numerical bias in later calculations) to the target sequence (E-value>e⁻¹⁰⁰) are selected. Preferably, to avoid potential bias, an even distribution of sequences among the entire range of sequence homology/identity is selected, if possible.

The BLAST programs, including BLASTp, are described in detail in the NCBI BLAST website (http://www.ncbi.nlm.nih.gov/BLAST/). The website also provides a BLAST Program Selection Guide to help a user in selecting the most suitable BLAST programs for specific needs and specific query sequences. The website also links to a number of protein and polynucleotide databases that can be search by the BLAST programs. The entire contents of the NCBI Blast website are incorporated herein by reference.

In performing a BLAST search, default parameters provided by the program can generally be used. However, in certain situations, user may opt to change certain alternative parameters (e.g., by selecting among many pull-down menus or providing numeric values in parameter fields) as offered by the program to suit specific needs.

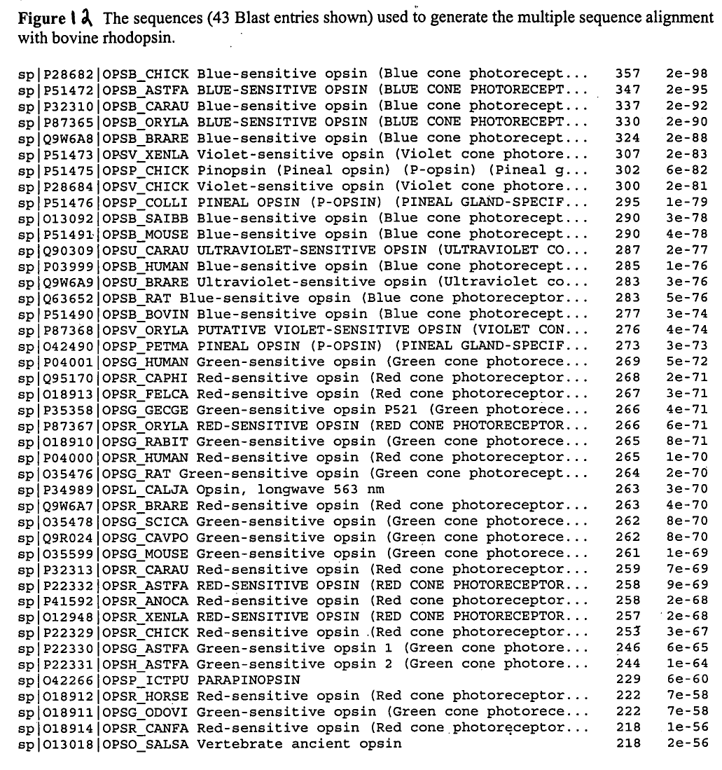

In selecting hit sequences for subsequent multi-sequence alignment, the method prefers an ensemble of sequences providing a uniform distribution of sequence identities from 20% to 100%, preferably 20-90%, or 35 to 90%. In other words, selecting clusters of, or disproportional numbers of homologous sequences with very similar values in sequence identity (e.g., all about 50-55% identical) are disfavored if possible. For a typical target sequence, about 20-100 sequences, preferably about 30-80, or 40-50 sequences are selected. In the case of bovine rhodopsin, 43 homologous sequences may be selected (see FIG. 12).

These homologous sequences, plus the target sequence are then used in a program such as ClustalW (Thompson et al., 1994, the entire contents of which incorporated herein by reference) to generate a multiple sequence alignment. This sequence alignment preferably includes sequences with identities to the target sequence as low as about 20%, 35%, 40%, 45%, or about 50%. In general, the method may include sequences with higher or lower non-zero E-values, but including too low a homology (higher E-values) might lead to additional alignment problems.

In an exemplary embodiment, such a step can be performed using the SeqHyd hydrophobic profile algorithm, which is based on peak signal analysis of the hydrophobic profile. SeqHyd requires a multiple sequence alignment using sequences related to the target sequence (e.g. bovine rhodopsin).

Step 1b: Average consensus hydrophobicity and initial TM assignment

In one embodiment of the invention, where no multisequence alignment is performed, the amino acid sequence can be directly converted, according to the chosen hydrophobicity scale, to hydrophobicity values at each residue positions.

In other embodiments where a multisequence alignment is performed, the equivalent step is to calculate the consensus hydrophobicity for every residue position in the alignment. This consensus hydrophobicity is the average hydrophobicity (e.g., using the Eisenberg hydrophobicity scale) of all the amino acids in that position over all the sequences in the multiple sequence alignment, optionally weighted for the frequency of occurrence of each particular amino acid at that position.

To plot a hydrophobic profile of the sequence or the aligned sequences, the method calculates the average hydrophobicity over a chosen window size (WS) of residues around every residue position, using WS ranging from 12 to 20.

For each residue, the window of calculation is preferably symmetric (with equal number of residues to the N- and C-terminal sides of the residue in question), although it is not impossible under certain circumstances to use asymmetric windows, which extend to different numbers of residues to the N- and C-terminal sides of the residue in question.

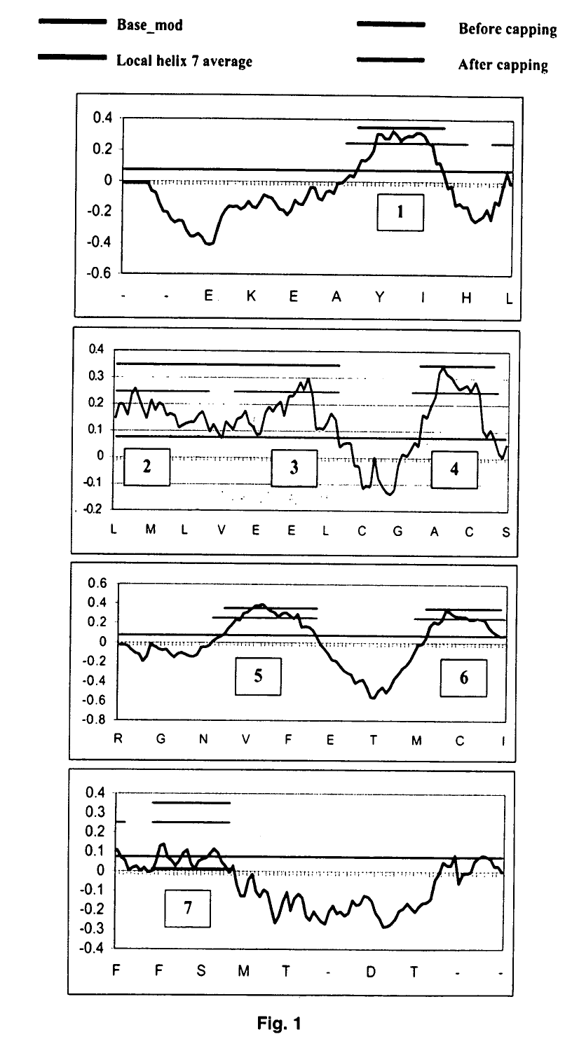

This average value of hydrophobicity at each sequence position is plotted to yield the hydrophobic profile (see, for example, FIG. 1 for WS=14 in the rhodopsin example). The baseline for this profile serves as the threshold value for determining the TM regions and is calculated as follows.

First, the method obtains the global average hydrophobicity value over all residues in the protein. In some embodiments, the method may include a step that excludes the solvent-exposed regions of the sequences, such as the amino terminus region (e.g., 34 residues in rhodopsin) and/or the carboxyl terminus region (e.g., 42 residues in rhodopsin) that are outside the TM regions. In certain embodiments, the solvent-exposed regions may also include some inter-TM region loops, especially those large loops with more than about 20 amino acids. These regions are accessible to solvent (e.g. water) on the intracellular or extracellular region, and tend to have non-insignificant numbers of hydrophilic residues that would skew the global average value.

This global average established a baseline that can be used for determining initial TM regions (peaks on the hydrophobic profile plot). If this baseline thus obtained does not resolve the expected number of peaks, then the method automatically changes the baselines over a range of a pre-determined value, e.g., 0.05, from the global average. The baseline closest to the average that yields the expected number of (seven for GPCR) peaks (“base_mod”) is used for TM region prediction. The base_mod provides the basis for the accurate determination of the TM regions in the sequence. This final baseline may be interpreted physically as a ΔG=0 value above which residues are thermodynamically stable in the transmembrane and below which they are not. This baseline is unique to the particular protein to which it is being applied, with its individual environmental factors (water clusters, ions, hydrophobic or hydrophilic ligand or interhelical interactions, membrane composition) that may change the relative stability of any particular residue.

Below WS=12, the fluctuations in hydrophobicity (noise) tend to be too large to be useful. The lowest WS that yields seven peaks (with peak length >10 and <50, and peak area >0.8) is denoted as WSₘᵢₙ. The peaks ranges for WSₘᵢₙ are used as input for the helix-capping module discussed in the next section.

In certain embodiments, assigning the TM region to certain short helices may be a problem, because they have shorter lengths and lower intensity peak hydrophobicity compared with all the other helices. This has been observed for some GPCRs (Vaidehi et al., 2002), especially helix 7 of the bovine rhodopsin. In that case, the low intensity of helix 7 arises because it has fewer highly hydrophobic residues (Ile, Phe, Val, and Leu) and because it has a consecutive stretch of hydrophilic residues (e.g., KTSAVYN, SEQ ID NO: 21). These short stretches of hydrophilic residues (including Lys-296) are involved in the recognition of the aldehyde group of 11-cis-retinal in rhodopsin. For such cases, the method uses the local average of the hydrophobicity (defined as a line from minimum to minimum around this peak, or the average hydrophobicity of all residues excluding those solvent-exposed N-terminal, C-terminal, and/or loop residues) as the baseline for assigning the TM predictions.

In a preferred embodiment, the method automatically applies this additional criteria when the peak length is <23, the peak area is <0.8, and the local average is >0.05 less than the base_mod. In other words, this local average is automatically applied for proteins where the residues are relatively hydrophilic but in which the helix might still be stable because of local environmental factors (mentioned above) that stabilize these residues.

Step 1c: Helix capping

It is possible that the actual length of the helix would extend past the membrane surface. Thus, the method provides an optional step to fine-tune the N- and C-terminal ends of the predicted TM helical regions. This helix -capping step is aimed at capping each helix at the top and bottom of the TM domain, and is premised on properties of known helix breaker residues. In addition, the method restricts the procedure so as not to extend the predicted TM helical region more than six residues.

In certain preferred embodiments, potential helix breakers include P and G; positively charged residues R, H, and K; and negatively charged residues as E and D.

In this step, the method first searches up to four residues from the edge going inwards from the initial TM prediction obtained from the previous section for a helix breaker. If it finds one, then the TM helix edges are kept at the initial values. In an alternative embodiment, the TM helix edges are broken off at the helix breaker, either including or excluding the helix breaker.

However, if no helix breaker is found, then the TM helical region is extended until a breaker is found, but with the restriction that the helix not be extended more than six residues on either side. Again, the TM helix edges are broken off at the helix breaker, either including or excluding the helix breaker.

In certain embodiments, regardless of capping step, the final shortest helical assignment allowed is 21, corresponding to the shortest known helical TM region. This lower size limit prevents incorporation of narrow noise peaks into TM helical predictions.

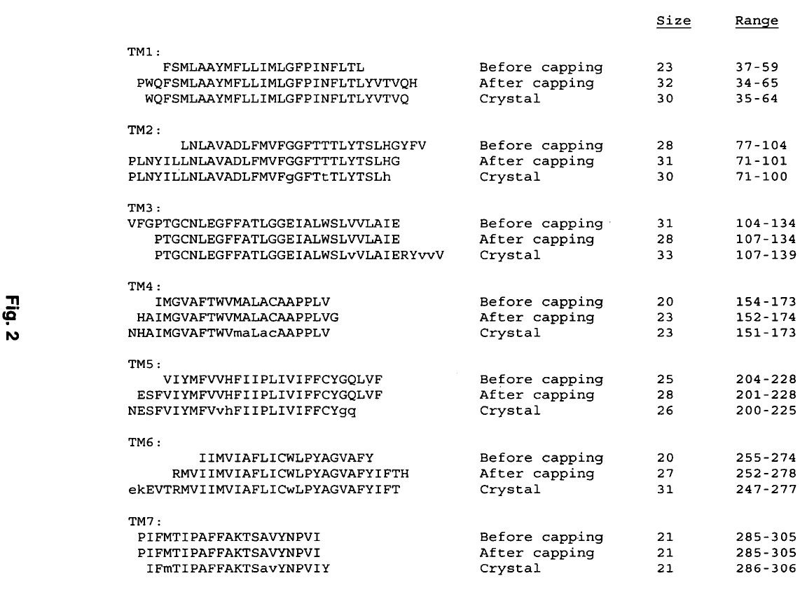

This algorithm has been successful used for predicting the structure for about 10 diverse GPCR classes (Vaidehi et al., 2002). In each case, the predicted binding site and binding energy agrees well with available experimental data, providing some validation of the TM helical region prediction. However, only for bovine rhodopsin have precise comparisons to an experimental structure been made (see below). For example, FIG. 2 compares the predictions of TM helical regions for bovine rhodopsin to the TM helical regions as assigned in the crystal structure (Palczewski et al., 2000).

In certain embodiments, the defined TM helical regions can be further checked to determine which residues have an αx-helical conformation. For this, the method analyses the Φ-Ψ angles using, for example, PROCHECK (Laskowski et al., 1993, incorporated herein by reference), and considers the experimental structure to be in an α-helix if −37<Φ<-77 and -27<Ψ<-67. This may lead to slightly shorter helices than quoted in the crystal structure (see the lowercase letters in FIG. 2, which indicate residues which are outside the above range but quoted as helices in the experimental article).

A specific embodiment of the subject transmembrane region-predicting method is in the form of the TM2ndS program, which is used to predict TM regions in Trabanino et al., Biophysical Journal 86: 1904-1921, 2004, the entire content of which is incorporated herein by reference.

Step 2: Assembly and optimization of the (seven-helical) TM bundle

Having predicted the TM domains using a TM2ndS-like method, the 3D structure prediction method next build them into the TM bundle (in the case of GPCR, seven-helical TM bundle). This generally involves two steps: 1) assembly and optimization of the relative translation, and, 2) rotation of the TM regions.

Step 2a: Assembly into a TM bundle

Canonical right-handed α-helices or β-sheets are built for each TM region using extended side-chain conformations (see above). Then the axes of the TM regions are oriented in space according to an electron density map of a homologous protein, or similar data providing information about the 3-D orientation of the TM regions, preferably from a homologous protein. For example, in the case of a putative GPCR, the 7.5 Å electron density map of frog rhodopsin (Schertler, 1998, incorporated by reference) can be used. This 7.5 Å electron density map gives only the rough relative orientations of the helical axes, with no data on atomic positions. This serves as the starting point for optimization of the helices in the helical bundle. In addition, the solved crystal structure of bovine rhodopsin may also provide the general orientation of individual TM regions in 3-D. The predicted, and optionally verified structures of other transmembrane proteins (such as a predicted structure of GPCR or ion channel) may also provide the same information regarding TM region orientation in 3-D. In all these cases, it is emphasized that no information as to helical translations or rotations is used.

In the case of GPCR and frog rhodopsin, since this electron density map showed no retinal present, it is not clear whether this form of rhodopsin is active or inactive. This same information has been used to build structures of about 10 other GPCR classes (Vaidehi et al., 2002). In each case the predictions of binding site and binding energy agrees well with available experimental data, providing some validation for this general approach of constructing the TM bundle of GPCRs. However, for bovine rhodopsin, much more precise comparisons to the experimental structures can be made, as reported below. Step 2b: Optimization of the relative translation of the TM regions in the bundle The translational and rotational orientation of each TM region in the TM bundle is critical to the nature and conformation of the binding site in the target TM protein (e.g. GPCR). The subject method does not use homology modeling to predict these quantities, especially in the case of GPCR, since many GPCRs have very remote sequence homology to the only available crystal structure—rhodopsin (sequence homology ranging down to 10%), making it quite difficult and risky to base a three-dimensional structure on homology modeling using the rhodopsin crystal structure as template. Also the subject method does not use atomistic molecular dynamics and molecular mechanics methods to optimize the structure, because the large barriers between various favorable positions can trap the conformation in local minima, making such approaches ineffective in repositioning the TM regions.

Instead, the subject method optimizes the initial packing by translating and rotating the TM regions over a grid of positions and by using various properties of the amino acids in the sequence to suggest initial starting points. This Monte Carlo-like systematic conformational search algorithm for rotational and translational orientation of the TM regions allows the system to surmount barriers in the conformational space. In addition, each TM region within the assembled TM bundle may individually has several favored translational and rotational conformations. By combining different conformations of different TM regions within the bundle, an ensemble/grid of potentially favorable TM bundles in membrane environment may be created and screened upon to identify the best one or few 3-D structures.

One of the general principles in repositioning the TM regions is that the outer surface of the TM bundle (at least the middle regions) should be hydrophobic to have stabilizing interactions with the hydrophobic chains of the lipid. The subject method define a midpoint plane through the lipid bilayer, corresponding to the contact of the hydrophobic chains, as the lipid midpoint plane (LMP). It is assumed that the hydrophobic regions of the TM bundle will position themselves such that the middle of their maximum hydrophobicity lies in this plane. Then the subject method determines the hydrophobic center (HC) for each TM region as the maximum of the peak of hydrophobicity from the hydrophobicity profiles generated with various window sizes.

This concept was tested and verified for the crystal structure of bovine rhodopsin below. The criterion for the best-fit to experiment is that these TM helical positions, when applied to the crystal structure, would all lie in a single plane that could be taken as the LMP.

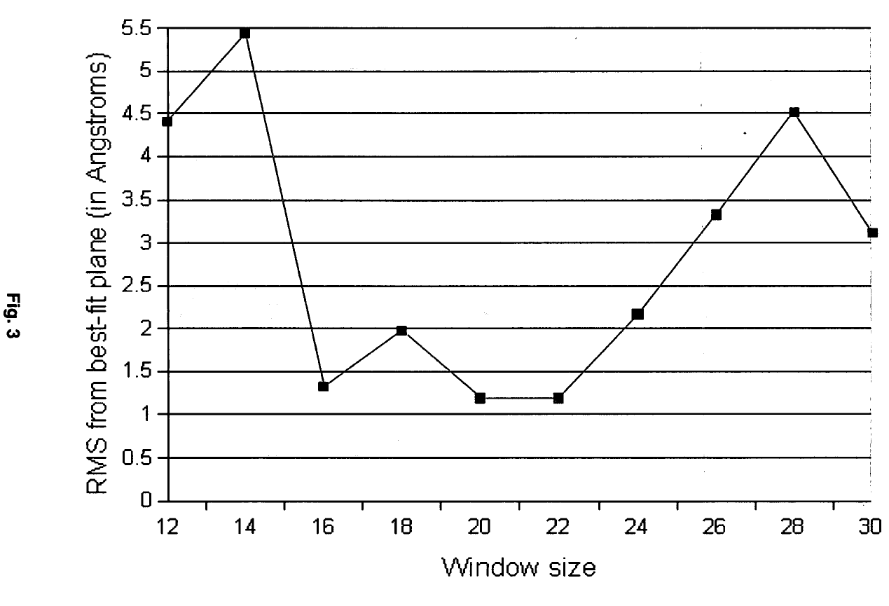

As shown in FIG. 3, the deviation of the calculated hydrophobic centers from lying in a single plane in the rhodopsin crystal structure is a minimum for WS 20 and 22. Thus a program such as Get_Centers calculates the overall hydrophobic center of each TM helix based on the average of centers obtained for a range of window sizes near 20. Get_Centers determines this range of window sizes as follows. First, each hydrophobic center (HC) is calculated for WS=20. Then, the HCs are calculated for WS 12-30 (excluding WS=20). For each helix Get_Centers determines the window sizes that yield HC less than five residues from the HC calculated at WS=20.

For example, consider helix 1 in Table 1. Here HC=18 for WS=20. For windows sizes 12, 14, 16, 18, 22, 24, 26, 28, and 30, the method finds HC=15, 13, 20, 18, 17, 18, 15, 16, and 13. For WS 16, 18, 22, and 24, the HC are less than five from the value at WS=20. Thus the hydrophobic center calculation is stable within this regime of window sizes. The HC calculated for WS 16, 18, 22, and 24 for the helices 2-7 are also less than five residues from the centers at WS=20. Thus, Get_Centers averages the HC for window sizes 16, 18, 20, 22, and 24 to calculate HC. Get_Centers takes these values (last column of Table 1) as the final TM helix centers. For bovine rhodopsin, these seven HCs deviate by a root mean-square (RMS) of 1.04 Å from a common plane.

TABLE 1
Window size

| | | | | | | | | | | | |
|---|---|---|---|---|---|---|---|---|---|---|---|
| Helix number | 12 | 14 | 16 | 18 | 20 | 22 | 24 | 26 | 28 | 30 | HC |
| 1 | 15 | 13 | 20 | 18 | 18 | 17 | 18 | 15 | 16 | 13 | 18.2 |
| 2 | 20 | 12 | 12 | 14 | 15 | 15 | 14 | 22 | 19 | 20 | 14.0 |
| 3 | 19 | 20 | 17 | 18 | 15 | 16 | 15 | 12 | 11 | 12 | 16.2 |
| 4 | 9 | 9 | 10 | 15 | 12 | 13 | 13 | 12 | 11 | 17 | 12.6 |
| 5 | 15 | 19 | 13 | 12 | 14 | 16 | 16 | 17 | 16 | 15 | 14.2 |
| 6 | 8 | 9 | 11 | 11 | 13 | 14 | 14 | 15 | 16 | 17 | 12.6 |
| 7 | 19 | 4 | 17 | 15 | 14 | 14 | 13 | 12 | 11 | 10 | 14.6 |

The last column shows the positions of the hydrophobic center (HC) predicted for each TM for various window sizes. The first row (in boldface) has the window sizes chosen to calculate this hydrophobic center (underlined). Here position 1 corresponds to the first residue in the capped sequence in FIG. 2.

Step 2c: Optimization of the Rotational Orientation

Once the TM regions are aligned along their longitudinal axes according to the calculated hydrophobic centers, the rotational orientation of the TM regions is optimized using either or both of the following steps.

Orienting the net hydrophobic moment of each TM region to point toward the membrane (phobic orientation): In this procedure (denoted as CoarseRot-H), the helical face with the maximum hydrophobic moment is calculated for the middle section of each TM region, denoted as the hydrophobic midregion (HMR). The face is the sector angle obtained as follows using TM as an example: 1) The central point of the sector angle is the intersection point of the helical axis (the active helix that is being rotated) with the common helical plane (LMP) and 2), the other two points forming the arc, are the nearest projections (on the LMP) of the C_{α} vectors of the two adjacent helices. The calculation of the hydrophobic moment vector is restricted to this face angle. This allows the predicted hydrophobic moment to be insensitive to cases in which the interior of the helix is uncharacteristically hydrophilic (because of ligand or water interaction within the bundle). In a preferred embodiment, HMR is chosen to be the middle 15 residues of each helix straddling the predicted hydrophobic center and exhibiting large hydrophobicity. This hydrophobic moment is projected onto the common helical plane (LMP) and oriented exactly opposite to the direction toward the geometric center of the TM barrel (GCB). In case of GPCR, this criterion is most appropriate for the six helices (excluding TM3) having significant contacts with the lipid membrane. The LMP is the plane that most closely intersects the hydrophobic centers as described in Step 2b. The GCB is calculated as the center of mass of the positions of the a-carbons for each residue in the HMR for each helix summed over all seven. This procedure is called phobic orientation.

Optimization of the Rotational Orientation Using Energy Minimization Techniques

(RotMin): In this procedure, each of the seven TMs is optimized through a range of rotations and translations one at a time (the active TM) while the other six TMs are reoptimized in response. After each rotation of the main chain (kept rigid) of each TM region, the side-chain positions of all residues for all seven helices of the GPCR in the TMR, for example, are optimized. In a preferred embodiment, SCWRL (Bower et al., 1997, incorporated herein by reference) can be used for this purpose. The potential energy of the active helix is then minimized (for up to 80 steps of conjugate gradients minimization until an RMS force of 0.5 kcal/mol per Å is achieved) in the field of all other helices (whose atoms are kept fixed). This procedure is carried out for a grid of rotation angles (typically every 2-10°, preferably 5°, for a range of ±50° to ±100°) for the active helix to determine the optimum rotation for the active helix. Then the active helix is kept fixed in its optimum rotated conformation, and each of the other helices are allowed to be rotated and optimized. Here the procedure for each of the remaining helices one by one is: 1) rotate the main chain; 2) placing and optimizing the side chains using a program such as SCWRL or SCAP; and 3) minimize the potential energy of all atoms in the helix. The optimization of these remaining helices is done iteratively until the entire grid of rotation angles is searched. This procedure is called RotMin.

In case of GPCR, this method is most important for TM3, which is near the center of the GPCR TM barrel and not particularly amphipathic (it has several charged residues leading to a small hydrophobic moment). For bovine rhodopsin, phobic orientation were used for placing the hydrophobic moments away from the GCB for all seven helices. Subsequently rotations were optimized using RotMin for helices 3 and 5 using small rotation angles of ±2.5°, ±5.0°, and ±8.0°. This optimizes the only salt bridge in the TM region (between residues His-211 and Glu-122). Coarse-grain rotation optimization combining both the energy optimization and hydrophobic moments is expected to provide better optimized TM helices than either one alone.

Step 3: Optimizing the Individual TM Regions

The optimization of the rotational and translational orientation of the TM regions described in the above steps is performed initially on canonical helices. In certain embodiments, these optimization steps are applied again to the helices and beta-barrels after their optimizations described in Step 3). To obtain a valid description of the backbone conformation for each residue in the helix, including the opportunity of G, P, and charged residues to cause a break in a helix, the helices built from the Step 2 were optimized separately. In this procedure, SCWRL is used for side-chain placement, then molecular dynamics (MD) (either Cartesian or torsional MD called NEIMO; Jain et al., 1993; Mathiowetz et al., 1994; Vaidehi et al., 1996, all incorporated herein by reference) simulations are carried out, for example, at 300 K for 500 ps. The structure with the lowest total potential energy in the last 250 ps is chosen and minimized using conjugate gradients.

This optimization step is important to correctly predict the bends and distortions that occur in the helix due to helix breakers such as proline and the two glycines. The MD also carries out an initial optimization of the side-chain conformations, which is later further optimized within the bundle using Monte Carlo side-chain replacement methods. This procedure allows each helix to optimize in the field due to the other helices in the optimized TM bundle from Step 2.

Step 4: Addition of Lipid Bilayer and Fine-Grain Reoptimization of the TM Bundle

To the final structure from Step 3, the subject method adds two layers of explicit lipid bilayers. For example, in the case of GPCR, the explicit lipid bilayers consist of 52 molecules of dilauroylphosphatidylcholine lipid around the TM bundle of seven helices. This step is done by inserting the TM bundle into a layer of optimized bilayer molecules in which a hole was built for the helix assembly and eliminating lipids with bad contacts (atoms closer than 10 Å). Then the quaternion-based rigid-body molecular dynamics (RB-MD) in MPSim (Lim et al., 1997) is used to carry out RB-MD for 50 ps (or until the potential and kinetic energies of the system stabilized). In this RB-MD step the helices and the lipid bilayer molecules are treated as rigid bodies, and 1-fs time steps at 300 K can be used. This RB-MD step is important to optimize the positions of the lipid molecules with respect to the TM bundle and to optimize the vertical helical translations, relative helical angles, and rotations of the individual helices in explicit lipid bilayers.

Step 5: Loop Building

Following the RB-MD, the method adds loops to the helices using the WHATIF software (Vriend, 1990, incorporated herein by reference) or any equivalents. After the addition of loops, SCWRL (Bower et al., 1997, supra) may be used to add the side-chains for all the residues. The loop conformations are then optimized by conjugate gradient minimization of the loop conformations while keeping the TM helices fixed.

This step also allows the general option of forming selected disulfide linkages. For example, in the case of GPCR, disulfide bond is formed between the cysteines in the EC-II loop, which are conserved across many GPCRs, and the N-terminal edge of TM3 or EC3. In the case of bovine rhodopsin, the alignment of the 44 sequences from Step 1, Sequence Alignment, indicates only one pair of fully conserved cysteines on the same side of the membrane (extracellular side). The disulfide bond was formed and optimized with equilibrium distances lowered in decrements of 2 Å until the bond distance was itself 2 Å.

Once the disulfide bond(s) are formed, the loops are optimized with the default equilibrium disulfide bond distance of 2.07 Å. Annealing MD can be used to optimize the (EC-II) loops at this stage. In the case of bovine rhodopsin, this involved 71 cycles, in each of which the loop atoms were heated from 50 K to 600 K and back to 50 K over a period of 4.6 ps. During the optimization process, the rest of the atoms were kept fixed for the first 330 ps and then the side chains within the cavity of the protein in the vicinity of the EC-II loop were allowed to move for 100 ps to allow accommodation of the loop.

Step 6: Full-Atom Optimization

Subsequently a full-atom conjugate gradient minimization of the protein is performed in vacuum using MPSim (Lim et al., 1997). This leads to the final MembStruk-predicted structure for the target TM protein.

(C) Function Prediction Using HierDock

Since there are no experimental structures available for any human GPCR, the only validation available for the accuracy of predicted structures for human GPCRs is to predict the ligand binding sites and the ligand binding energies. The accuracy in the predicted binding site can then be judged from site-directed mutagenesis experiments on the residues predicted to control selectivity. An even tougher test is to compare binding affinity of ligands to each other and to mutated proteins. For many GPCRs of pharmaceutical interest there is ample experimental data on ligand binding constants as well as agonist and antagonist inhibition constants for many GPCRs (for a compilation of this literature, see http://www.gpcr.org).

Such a general approach can be equally well applied to other predicted TM protein structures, if such TM proteins are known to bind certain ligands (including other proteins, small molecule lipids, steroids, nucleotides, ions, etc.).

To carry out such function validations for the predicted structures, it is essential to have reliable and efficient procedures for predicting binding site and binding affinities. Since the ligand binding site is completely unknown for most GPCRs and many other TM proteins, the subject method scan the entire protein to identify likely binding sites and conformation of each ligand. Then, the method reliably ranks the relative binding energies of the various ligands in these sites. The improved HierDock procedure, which has been tested and validated for predicting ligand binding sites and ligand binding energies for many globular and membrane-bound proteins, can be used for this purpose. The multistep hierarchical procedure in HierDock, ranging from coarse-grain docking to fine-grain MD optimization, leads to efficient and accurate predictions for ligand binding in proteins.

U.S. Patent Application Publication No. US20020099506A1 and PCT publication WO0171347A1 (both incorporated herein by reference) describe a previous version of HierDock, which has been used extensively to predict and verify the binding of amino acids to amino acyl tRNA transferase and other ligand-protein interactions, including binding of odorants to membrane-bound olfactory receptors (Floriano et al., P.N.A.S. U.S.A. 97: 10712-6, 2000), binding of outer membrane protein A to sugars (Datta et al., J. Am. Chem. Soc. 124: 5652-5653, 2002) (also see Datta et al., P.N.A.S. U.S.A. 99: 2636-41, 2002; and Datta et al., Proteins 50(2): 213-21, Feb. 1, 2003).

Briefly, the HierDock ligand screening protocol follows a hierarchical strategy for examining conformations, binding sites and binding energies. Such a hierarchical method has been shown to be necessary for docking algorithms (Halperin et al., Proteins: Struct. Funct. & Gene. 47, 409-443, 2002). The steps in HierDock generally involve using coarse-grain docking methods to generate several conformations of protein/ligand complexes followed by molecular dynamics (MD) simulations including continuum solvation methods performed on a subset of good conformations generated from the coarse-grain docking.

The three major steps in HierDock procedure are briefly described as follows:

First, a coarse-grain docking procedure is used to generate a set of conformations for ligand binding. For example, DOCK 4.0 (Ewing and Kuntz, J. Comput. Chem. 18: 1175-1189, 1997; Ewing et al., J. Comput. Aid. Mol. Design 15: 411-428, 2001, incorporated herein by reference) can be used to generate and score 20,000 configurations, of which 10% were ranked using the DOCK scoring function.

Second, the 20 best conformations for each ligand from DOCK are selected, and subjected to annealing molecular dynamics (MD) to further optimize the conformation in the local binding pocket, allowing the atoms of the ligand to move in the field of the protein. In this step, the system may be heated and cooled from, for example, 50 K to 600 K, in steps of, for example, 10 K (0.05 ps at each temperature) for one cycle. At the end of annealing MD cycle, the best energy structure is retained. Annealing MD allows the ligand to readjust in the binding pocket to optimize its interaction with the protein. This fine grain optimization may be performed using MPSim (Lim et al., J. Comput. Chem. 18: 501-521, 1997) with DREIDING force field (supra) and continuum solvation methods, or any other similar methods. Surface Generalized Born (SGB) continuum solvent method can be used to obtain forces and energies resulting from the polarization of the solvent by the charges of the ligand and protein. This would allow calculation of the change in the ligand structure due to the solvent field and hence, obtain more realistic binding energies that take into account the solvation effects on the ligand/protein structure. The annealing MD procedure will generate 20 protein/ligand complexes for each ligand.

Third, for the 20 structures generated by annealing MD simulations for each ligand, the potential energy of the full ligand/protein complex in aqueous solution can be minimized (using, for example, conjugate gradients minimization) using SGB. his step of protein/ligand-complex optimization is critical for obtaining energetically good conformations for the complex (cavity+ligand). Binding energies as the difference between the total energy of the ligand-protein complex in solvent (ΔG_{(protein+ligand)}), the sum of the total energies of the protein (ΔG₍ₚᵣₒₜₑᵢₙ₎), and the ligand separately in solvent (ΔG_{(ligand)}) can then be calculated. The energies of the protein and the ligand in solvent are calculated after independent energy minimization of the protein and the ligand separately in water. Solvation energies can be calculated using Poisson-Boltzmann continuum solvation method available in the software suite Delphi. The non-bond interaction energies can be calculated exactly using all pair interactions. Thus the binding energy is given by:
ΔΔG_{calc}=ΔG_{(protein+ligand)}−ΔGₚᵣₒₜₑᵢₙ−ΔG_{ligand} (1)

Since the structure optimizations included solvation forces using the SGB continuum solvent approximation with the experimental dielectric constant, the calculated energies can be considered free energies (Hendsch and Tidor, Protein Sci. 8: 1381-92, 1999). This multi-step scanning procedure is based on docking via DOCK 4.0 coupled with fine-grain MM techniques. The coarse grain docked complex structures generated are scored with FF and differential salvation, which effectively filters the docked complexes to isolate the top contenders.

Next, the potential energy of the target protein-ligand complex is minimized using any of the suitable methods, such as DREIDING force field in MPSim (Lim et al., J. Comput. Chem. 18: 501-521, 1997). Optionally, Poisson-Boltzmann dielectric continuum solvent was included to simulate solvation in water.

The various steps involved in this current procedure are as follows:

Step 1: Sphere Generation

The method assumes no knowledge of the ligand binding site in the TM protein (e.g. GPCRs), and hence the entire molecular surface of the target TM protein is scanned to predict the energetically preferred ligand binding sites. The negative of the molecular surface of the protein was used to define potential binding regions within the target protein over which the various ligand conformations are to be sampled. The void regions are mapped with spheres generated over the whole target protein using the Sphgen program in DOCK (see Kuntz laboratory at UCSF, San Francisco, Calif.).

Sphgen generates sets of overlapping spheres to describe the shape of a molecule or molecular surface (Kuntz et al., J. Mol. Biol. 161: 269-288, 1982; DesJarlais et al., J. Med. Chem. 31(4): 722-729, 1988, all incorporated by reference). For receptors, a negative image of the surface invaginations is created; for a ligand, the program creates a positive image of the entire molecule. Spheres are constructed using the molecular surface described by Richards (Ann. Rev. Biophys. Bioeng. 6: 151-176, 1977, incorporated by reference) calculated with the program MS (Connolly, J. Appl. Cryst. 16: 548-558, 1983; Science. 221: 709-713, 1983, all incorporated by reference). Each sphere touches the molecular surface at two points and has its radius along the surface normal of one of the points. For the receptor, each sphere center is “outside” the surface, and lies in the direction of a surface normal vector. For a ligand, each sphere center is “inside” the surface, and lies in the direction of a reversed surface normal vector. Spheres are calculated over the entire surface, producing approximately one sphere per surface point. This very dense representation is then filtered to keep only the largest sphere associated with each receptor surface atom. The filtered set is then clustered on the basis of radial overlap between the spheres using a single linkage algorithm. This creates a negative image of the receptor surface, where each invagination is characterized by a set of overlapping spheres. These sets, or “clusters,” are sorted according to numbers of constituent spheres, and written out in order of descending size. The largest cluster is typically the ligand binding site of the receptor molecule. The program showsphere writes out sphere center coordinates in PDB format and may be helpful for visualizing the clusters.

Although SPHGEN is a preferred embodiment (since it captures shape information about the site of interest very well), it is not necessary for this step. Any method which results in points throughout the site could be used. For example, the coordinates of a known ligand can be used; points along a solvent accessible surface of the receptor can be used; a grid of points in the site can also be used. Alternatives that take into account the chemistry of the site are also possible, for example, the GRID program can be used. Interaction “hotspots” generated by this program can be used as surrogate sphere centers.

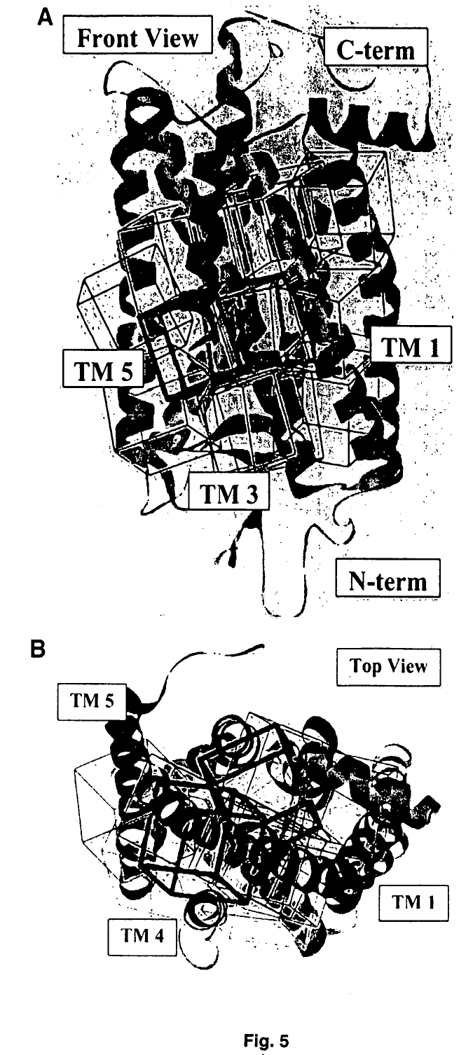

No assumptions were made on the nature or the location of the binding site in these target proteins. For bovine rhodopsin, this led to a total of 7474 spheres, which was partitioned into 13 overlapping docking regions each with a volume of 10 Å³ as shown in FIG. 5. For some target proteins, if it is known that certain areas of the protein are involved in contact with molecules other than the ligand of interest, such as the membrane lipids or near the intracellular region likely to be involved in binding to other effector molecules (e.g. G-proteins, etc.), such areas are excluded from potential docking regions. Regardless, in this step, no assumptions are made on either the nature or the location of the binding site in these regions.

Step 2. Coarse-Grain Sampling

To locate the most favorable ligand binding site(s), DOCK 4.0 (Ewing and Kuntz, 1997, incorporated herein by reference) can be used to generate a set of conformations for binding the ligand of interest (e.g. in the case of rhodopsin, 11-cis-retinal—a ligand known to bind to bovine rhodopsin) to each of the overlapping docking regions identified above. For this docking step, a bump filter of 10, a non-distance-dependent dielectric constant of 1.0, and a cutoff of 10 Å may be used for energy evaluation. These values may be adjusted according to specific target proteins. The ligands are generally docked as nonflexible molecules to generate and score 100 conformations of the ligand in each of the overlapping docking regions. Any ligand conformation with, for example, <90% (or other user defined value) of the surface area buried into the protein are rejected, and the remainder is ranked by the ligand-protein interaction energy using DREIDING FF or other suitable FF. The best binding energy conformation among the overlapping docking regions is chosen as the putative binding region. Other conformations with binding energies within 100 kcal/mol of the best conformation can be chosen as possible binding regions.

Step 3. Construction of Putative Binding Region Using a More Refined Sampling of Ligand-Protein Interactions

A set of overlapping boxes are used to enclose the volume corresponding to the putative binding region (or regions) determined in Step 2, which is now to be used for a new sampling of ligand-protein conformations similar to Step 2.

Step 4. Coarse-Grain Sampling of Putative Binding Regions

To locate the most favorable ligand binding site(s), DOCK 4.0 is again used to generate a set of conformations for binding the ligand of interest (e.g. 11-cis-retinal) to the putative binding region. Again, a bump filter of 10, a non-distance-dependent dielectric constant of 1.0, and a cutoff of 10 Å may be used for energy evaluation. The ligands are docked as nonflexible molecules to generate and score, for example, 1000 conformations. The top 10% (i.e., 100) with best DOCK 4.0 score can be selected for further analysis.

Step 5. Ligand-Only Minimization

The 100 best conformations selected from Step 4 are conjugate-gradient-minimized, keeping the protein fixed but all atoms of the ligand movable. Minimized ligand conformations that satisfied the buried surface area cutoff criterion of about 75% are kept for the next step.

Step 6: Ligand-Protein Full Minimization

The ligand/protein conformations from Step 5 are further energy-minimized with all atoms (protein, lipid, and ligand) movable using conjugate gradients. The structure with the binding energy calculated by Eq. 1 is selected as:
BE₁=Energy_{[ligand in protein complex]}−Energy_{[free Ligand in solvent}] (Eq. 1)

Here the energy of the ligand in water is calculated using DREIDING FF and analytical volume Generalized-Born continuum solvation (GBCS) method. If a substantial part of the complex is in contact with the membrane environment, which is generally true for large transmembrane proteins, the complex need not (but can) be solvated.

Step 7. Side-Chain Optimization

Using the best binding conformation from Step 6, the side-chain conformations for all the residues within 5 Å of the bound ligand are optimized using any side-chain placement and optimization program, such as SCWRL or SCAP, etc. The resulting ligand-protein structure is finally optimized by conjugate gradient minimization allowing all atoms to relax.

Step 8. Iterative HierDock (Optional)

The protein from Step 7 (optimized with ligand bound) is saved. Steps 4-6 are repeated to obtain the best possible conformation for the ligand within the protein (with side-chains optimized in the presence of the ligand). This is an optional step.

(D) Transmembrane Proteins

The methods and apparatus of the invention are generally applicable to the prediction of three-dimensional structure of any membrane proteins, especially those proteins comprised predominantly of transmembrane structures, e.g. multi-pass transmembrane protein (such as various pumps, ion channels and transporters). The methods and apparatus are especially well-suited for those 7-TransMembrane (7-TM) proteins, such as G Protein-Coupled Receptors (GPCRs).

In certain embodiments, the transmembrane domains are mostly, or all alpha-helices.

In one embodiment, the methods of the invention are used to predict the secondary and tertiary structure of the transmembrane region of a transmembrane protein.

Thus the methods of the invention can be used for the 3-D structure prediction, and ligand binding (based on either the crystal structure or the predicted structure) for: G protein-coupled receptors, receptor tyrosine kinases, cytokine receptors, and ion channels. In certain embodiments the method described herein is used for predicting the structure of, and/or identifying ligands for “orphan receptors” for which no ligand is known.

In some embodiments, the receptor is a cell surface receptor, such as: a receptor tyrosine kinase, for example, an EPH receptor; an ion channel; a cytokine receptor; an multisubunit immune recognition receptor; a chemokine receptor; a growth factor receptor; or a G-protein coupled receptor, such as a chemoattracttractant peptide receptor; a neuropeptide receptor; a light receptor; a neurotransmitter receptor; or a polypeptide hormone receptor.

In a preferred embodiment, the receptor comprises 2 or more, preferrably 3 or more, 4 or more transmembrane domains. In a preferred embodiment, the receptor comprises 7 transmembrane domains.

G Protein-Coupled Receptors

GPCRs share a predicted seven-transmembrane helix structure and the ability to activate a G-protein (Gᵢ, G_{α}, Gₒ, etc.) in response to ligand binding. Their natural ligands range from peptide and non-peptide neurotransmitters, hormones, and growth factors to odorants and light. The members of the GPCR superfamily which act through heterotrimeric G-proteins have been classified into six clans, as set out below:

Classification of G-Protein Coupled Receptors

1. Clan A: rhodopsin like receptors

Family I: Olfactory receptors, adenosine receptors, melanocortin receptors and others.

Family II: Biogenic amine receptors.

Family III: Vertebrate opsins and neuropeptide receptors.

Family IV: Invertebrate opsins.

Family V: Chemokine, chemotactic, somatostatin, opioids and others.

Family VI: Melatonin receptors and others.

2. Clan B: calcitonin and related receptors

Family I: Calcitonin, calcitonin-like, and CRF receptors.

Family II: PTH/PTHrP receptors.

Family III: Glucagon, secretin receptors and others.

Family IV: Latrotoxin receptors and others.

3. Clan C: metabotropic glutamate and related receptors

Family I: Metabotropic glutamate receptors

Family II: Calcium receptors

Family III: GABA-B receptors

Family IV: Putative pheromone receptors

4. Clan D: STE2 pheromone receptors

5. Clan E: STE3 pheromone receptors

6. Clan F: cAMP receptors

For example, one group of members of Clan A, Family I are the olfactory (odor) receptors (ORs) in the mammalian olfaction system. These receptors, unlike many other GPCRs that are designed for the specific recognition of few ligands, exhibit a combinatorial response to thousands of odorant molecules. See Malnic, B., et al. (1999) Cell 96, 713-723. Å single odor elicits response from multiple receptors and a single receptor also responds to multiple odorants, so every odorant has been thought to have a uni que combination of responses from several receptors. Odor detection is mediated by approximately 1,000 ORs that are G protein coupled membrane-bound proteins. Malnic et al. recently reported the differential responses of individual mouse OR neurons to 24 organic odor compounds (linear alcohols, acids, diacids, and bromoacids with four to nine carbons) by using Ca²⁺ imaging techniques, followed by single-cell reverse transcription-PCR to determine the sequence of the responsive OR. These results lead to the compelling question of “what is the molecular basis of odor recognition?” Such questions can be answered only with an understanding of the atomic-level structure of these ORs.

Most G protein-coupled receptors are comprised of a single protein chain that is threaded through the plasma membrane seven times. Such receptors are often referred to as seven-transmembrane receptors (STRs). More than a hundred different STRs have been found, including many distinct receptors that bind the same ligand, and there are likely many more STRs awaiting discovery. In addition, STRs have been identified for which the natural ligands are unknown; these receptors are termed “orphan” G protein-coupled receptors, as described above. Examples include receptors cloned by Neote et al. (1993) Cell 72, 415; Kouba et al. FEBS Lett. (1993) 321, 173; Birkenbach et al.(1993) J. Virol. 67, 2209.

The “exogenous receptors” may be any G protein-coupled receptor which is exogenous to a cell of interest. This receptor may be a plant or animal cell receptor. Studying plant cell receptors may be useful in the development of, for example, herbicides. In the case of an animal receptor, it may be of invertebrate or vertebrate origin. If an invertebrate receptor, an insect receptor is preferred, and would facilitate development of insecticides. The receptor may also be a vertebrate, more preferably a mammalian, still more preferably a human, receptor. The exogenous receptor is also preferably a seven transmembrane segment receptor.

Known ligands for G protein coupled receptors include: purines and nucleotides, such as adenosine, cAMP, ATP, UTP, ADP, melatonin and the like; biogenic amines (and related natural ligands), such as 5-hydroxytryptamine, acetylcholine, dopamine, adrenaline, adrenaline, adrenaline., histamine, noradrenaline, noradrenaline, noradrenaline., tyramine/octopamine and other related compounds; peptides such as adrenocorticotrophic hormone (acth), melanocyte stimulating hormone (msh), melanocortins, neurotensin (nt), bombesin and related peptides, endothelins, cholecystokinin, gastrin, neurokinin b (nk3), invertebrate tachykinin-like peptides, substance k (nk2), substance p (nk1), neuropeptide y (npy), thyrotropin releasing-factor (trf), bradykinin, angiotensin ii, beta-endorphin, c5a anaphalatoxin, calcitonin, chemokines (also called intercrines), corticotrophic releasing factor (crf), dynorphin, endorphin, fmlp and other formylated peptides, follitropin (fsh), fungal mating pheremones, galanin, gastric inhibitory polypeptide receptor (gip), glucagon-like peptides (glps), glucagon, gonadotropin releasing hormone (gnrh), growth hormone releasing hormone(ghrh), insect diuretic hormone, interleukin-8, leutropin (lh/hcg), met-enkephalin, opioid peptides, oxytocin, parathyroid hormone (pth) and pthrp, pituitary adenylyl cyclase activiating peptide (pacap), secretin, somatostatin, thrombin, thyrotropin (tsh), vasoactive intestinal peptide (vip), vasopressin, vasotocin; eicosanoids such as ip-prostacyclin, pg-prostaglandins, tx-thromboxanes; retinal based compounds such as vertebrate 11-cis retinal, invertebrate 11-cis retinal and other related compounds; lipids and lipid-based compounds such as cannabinoids, anandamide, lysophosphatidic acid, platelet activating factor, leukotrienes and the like; excitatory amino acids and ions such as calcium ions and glutamate.

Such ligands or derivatives thereof may be used in the HierDock protocol for prediction of ligand binding and mutagenesis study in silico. Such ligands may also be used as a library when screening for potential ligands for orphan GPCRs.

Suitable examples of G-protein coupled receptors include, but are not limited to, dopaminergic, muscarinic cholinergic, a-adrenergic, b-adrenergic, opioid (including delta and mu), cannabinoid, serotoninergic, and GABAergic receptors. Preferred receptors include the 5HT family of receptors, dopamine receptors, C5a receptor and FPRL-1 receptor, cyclo-histidyl-proline-diketoplperazine receptors, melanocyte stimulating hormone release inhibiting factor receptor, and receptors for neurotensin, thyrotropin releasing hormone, calcitonin, cholecytokinin-A, neurokinin-2, histamine-3, cannabinoid, melanocortin, or adrenomodulin, neuropeptide-Y1 or galanin. Other suitable receptors are listed in the art.

Specifically, preferred G protein-coupled receptors include α1A-adrenergic receptor, α1B-adrenergic receptor, α2-adrenergic receptor, α2B-adrenergic receptor, 1-adrenergic receptor, β2-adrenergic receptor, β3-adrenergic receptor, m1 acetylcholine receptor (AChR), m2 AChR, m3 AChR, m4 AChR, m5 AChR, D1 dopamine receptor, D2 dopamine receptor, D3 dopamine receptor, D4 dopamine receptor, D5 dopamine receptor, A1 adenosine receptor, A2b adenosine receptor, 5-HT1a receptor, 5-HT1b receptor, 5HT1-like receptor, 5-HT1d receptor, 5HT1d-like receptor, 5HT1d beta receptor, substance K (neurokinin A) receptor, fMLP receptor, fMLP-like receptor, angiotensin II type 1 receptor, endothelin ETA receptor, endothelin ETB receptor, thrombin receptor, growth hormone-releasing hormone (GHRH) receptor, vasoactive intestinal peptide receptor, oxytocin receptor, somatostatin SSTR1 and SSTR2, SSTR3, cannabinoid receptor, follicle stimulating hormone (FSH) receptor, leutropin (LH/HCG) receptor, thyroid stimulating hormone (TSH) receptor, thromboxane A2 receptor, platelet-activating factor (PAF) receptor, C5a anaphylatoxin receptor, Interleukin 8 (IL-8) IL-8RA, IL-8RB, Delta Opioid receptor, Kappa Opioid receptor, mip-1/RANTES receptor, Rhodopsin, Red opsin, Green opsin, Blue opsin, metabotropic glutamate mGluR1-6, histamine H2 receptor, ATP receptor, neuropeptide Y receptor, amyloid protein precursor receptor, insulin-like growth factor II receptor, bradykinin receptor, gonadotropin-releasing hormone receptor, cholecystokinin receptor, melanocyte stimulating hormone recepter, antidiuretic hormone receptor, glucagon receptor, and adrenocorticotropic hormone II receptor. The term ‘receptor,’ as used herein, encompasses both naturally occurring and mutant receptors.

The homology of STRs is discussed in Dohlman et al., Ann. Rev. Biochem., (1991) 60:653-88. When STRs are compared, a distinct spatial pattern of homology is discernible. The transmembrane domains are often the most similar, whereas the N- and C-terminal regions, and the cytoplasmic loop connecting transmembrane segments V and VI are more divergent.

Cytokine Receptors

In one embodiment, the target transmembrane protein is a cytokine receptor.

Cytokines are a family of soluble mediators of cell-to-cell communication that includes interleukins, interferons, and colony-stimulating factors. The characteristic features of cytokines lie in their functional redundancy and pleiotropy.

Most of the cytokine receptors that constitute distinct superfamilies do not possess intrinsic protein tyrosine kinase domains, yet receptor stimulation usually invokes rapid tyrosine phosphorylation of intracellular proteins, including the receptors themselves. Many members of the cytokine receptor superfamily activate the Jak protein tyrosine kinase family, with resultant phosphorylation of the STAT transcriptional activator factors. IL-2, IL-7, IL-2 and Interferon y have all been shown to activate Jak kinases (Frank et al (1995) Proc Natl Acad Sci USA 92:7779-7783); Scharfe et al. (1995) Blood 86:2077-2085); (Bacon et al. (1995) Proc Natl Acad Sci USA 92:7307-7311); and (Sakatsume et al (1995) J. Biol Chem 270:17528-17534). Events downstream of Jak phosphorylation have also been elucidated. For example, exposure of T lymphocytes to IL-2 has been shown to lead to the phosphorylation of signal transducers and activators of transcription (STAT) proteins STAT1α, STAT2β, and STAT3, as well as of two STAT-related proteins, p94 and p95. The STAT proteins were found to translocate to the nucleus and to bind to a specific DNA sequence, thus suggesting a mechanism by which IL-2 may activate specific genes involved in immune cell function (Frank et al. supra). Jak3 is associated with the gamma chain of the IL-2, IL-4, and IL-7 cytokine receptors (Fujii et al. (1995) Proc Natl Acad Sci 92:5482-5486) and (Musso et al (1995) J Exp Med. 181:1425-1431). The Jak kinases have also been shown to be activated by numerous ligands that signal via cytokine receptors such as, growth hormone and erythropoietin and IL-6 (Kishimoto (1994) Stem cells Suppl 12:37-44).

There are major families of cytokine receptors. Some are members of the Ig supergene family (e.g. IL-1 receptor), others are members of the nerve growth factor receptor family (e.g. TNF), but the majority are members of the haematopoietic growth factor family (e.g. IL-3, GM-CSF). Yet other cytokine receptors do not belong to a family, e.g. IFN-gamma. See Foxwell et al., Clin Exp Immunol. 1992 November;90(2):161-9.

Multisubunit Immune Recognition Receptor (MIRR)

In another embodiment, the methods of the invention can be used to predict the 3-D structure of a multisubunit receptor with multiple transmembrane domains. Receptors can be comprised of multiple proteins referred to as subunits, one category of which is referred to as a multisubunit receptor is a multisubunit immune recognition receptor (MIRR). MIRRs include receptors having multiple noncovalently associated subunits and are capable of interacting with src-family tyrosine kinases. MIRRs can include, but are not limited to, B cell antigen receptors, T cell antigen receptors, Fc receptors and CD22. One example of an MIRR is an antigen receptor on the surface of a B cell. To further illustrate, the MIRR on the surface of a B cell comprises membrane-bound immunoglobulin (mIg) associated with the subunits Ig-α and Ig- or Ig-γ, which forms a complex capable of regulating B cell function when bound by antigen. An antigen receptor can be functionally linked to an amplifier molecule in a manner such that the amplifier molecule is capable of regulating gene transcription.

Signal transduction pathways for MIRR complexes are capable of regulating the biological functions of a cell. Such functions can include, but are not limited to the ability of a cell to grow, to differentiate and to secrete cellular products. MIRR-induced signal transduction pathways can regulate the biological functions of specific types of cells involved in particular responses by an animal, such as immune responses, inflammatory responses and allergic responses. Cells involved in an immune response can include, for example, B cells, T cells, macrophages, dendritic cells, natural killer cells and plasma cells. Cells involved in inflammatory responses can include, for example, basophils, mast cells, eosinophils, neutrophils and macrophages. Cells involved in allergic responses can include, for example mast cells, basophils, B cells, T cells and macrophages. Thus the predicted structure of these MIRR complexes may be used for drug design to obtain agonists/antagonists that modulates the functions of these proteins, thereby regulating these biological processes.

Receptor Tyrosine Kinases

In still another embodiment, the methods of the invention can be used to predict the 3-D structure of a receptor tyrosine kinase, at least the transmembrane portion(s) thereof. The receptor tyrosine kinases can be divided into five subgroups on the basis of structural similarities in their extracellular domains and the organization of the tyrosine kinase catalytic region in their cytoplasmic domains. Sub-groups I (epidermal growth factor (EGF) receptor-like), II (insulin receptor-like) and the eph/eck family contain cysteine-rich sequences (Hirai et al., (1987) Science 238:1717-1720 and Lindberg and Hunter, (1990) Mol. Cell. Biol. 10:6316-6324). The functional domains of the kinase region of these three classes of receptor tyrosine kinases are encoded as a contiguous sequence (Hanks et al. (1988) Science 241:42-52). Subgroups III (platelet-derived growth factor (PDGF) receptor-like) and IV (the fibro-blast growth factor (FGF) receptors) are characterized as having immunoglobulin (Ig)-like folds in their extracellular domains, as well as having their kinase domains divided in two parts by a variable stretch of unrelated amino acids (Yanden and Ullrich (1988) supra and Hanks et al. (1988) supra).

The family with by far the largest number of known members is the EPH family. Since the description of the prototype, the EPH receptor (Hirai et al. (1987) Science 238:1717-1720), sequences have been reported for at least ten members of this family, not counting apparently orthologous receptors found in more than one species. Additional partial sequences, and the rate at which new members are still being reported, suggest the family is even larger (Maisonpierre et al. (1993) Oncogene 8:3277-3288; Andres et al. (1994) Oncogene 9:1461-1467; Henkemeyer et al. (1994) Oncogene 9:1001-1014; Ruiz et al. (1994) Mech Dev 46:87-100; Xu et al. (1994) Development 120:287-299; Zhou et al. (1994) J Neurosci Res 37:129-143; and references in Tuzi and Gullick (1994) Br J Cancer 69:417-421). Remarkably, despite the large number of members in the EPH family, all of these molecules were identified as orphan receptors without known ligands.

The expression patterns determined for some of the EPH family receptors have implied important roles for these molecules in early vertebrate development. In particular, the timing and pattern of expression of sek, mek4 and some of the other receptors during the phase of gastrulation and early organogenesis has suggested functions for these receptors in the important cellular interactions involved in patterning the embryo at this stage (Gilardi-Hebenstreit et al. (1992) Oncogene 7:2499-2506; Nieto et al. (1992) Development 116:1137-1150; Henkemeyer et al., supra; Ruiz et al., supra; and Xu et al., supra). Sek, for example, shows a notable early expression in the two areas of the mouse embryo that show obvious segmentation, namely the somites in the mesoderm and the rhombomeres of the hindbrain; hence the name sek, for segmentally expressed kinase (Gilardi-Hebenstreit et al., supra; Nieto et al., supra). As in Drosophila, these segmental structures of the mammalian embryo are implicated as important elements in establishing the body plan. The observation that Sek expression precedes the appearance of morphological segmentation suggests a role for sek in forming these segmental structures, or in determining segment-specific cell properties such as lineage compartmentation (Nieto et al., supra). Moreover, EPH receptors have been implicated, by their pattern of expression, in the development and maintenance of nearly every tissue in the embryonic and adult body. For instance, EPH receptors have been detected throughout the nervous system, the testes, the cartilaginous model of the skeleton, tooth primordia, the infundibular component of the pituitary, various epithelial tissues, lung, pancreas, liver and kidney tissues. Observations such as this have been indicative of important and unique roles for EPH family kinases in development and physiology, but further progress in understanding their action has been severely limited by the lack of information on their ligands.

As used herein, the terms “EPH receptor” or “EPH-type receptor” refer to a class of receptor tyrosine kinases, comprising at least eleven paralogous genes, though many more orthologs exist within this class, e.g., homologs from different species. EPH receptors, in general, are a discrete group of receptors related by homology and easily recognizable, for example, they are typically characterized by an extracellular domain containing a characteristic spacing of cysteine residues near the N-terminus and two fibronectin type III repeats (Hirai et al. (1987) Science 238:1717-1720; Lindberg et al. (1990) Mol Cell Biol 10:6316-6324; Chan et al. (1991) Oncogene 6:1057-1061; Maisonpierre et al. (1993) Oncogene 8:3277-3288; Andres et al. (1994) Oncogene 9:1461-1467; Henkemeyer et al. (1994) Oncogene 9:1001-1014; Ruiz et al. (1994) Mech Dev 46:87-100; Xu et al. (1994) Development 120:287-299; Zhou et al. (1994) J Neurosci Res 37:129-143; and references in Tuzi and Gullick (1994) Br J Cancer 69:417-421). Exemplary EPH receptors include the eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors. The term “EPH receptor” refers to the membrane form of the receptor protein, as well as soluble extracellular fragments which retain the ability to bind the ligand of the present invention.

Chemoattractant Receptors

The N-formyl peptide receptor is a classic example of a calcium mobilizing G protein-coupled receptor expressed by neutrophils and other phagocytic cells of the mammalian immune system (Snyderman et al. (1988) In Inflammation: Basic Principles and Clinical Correlates, pp. 309-323). N-Formyl peptides of bacterial origin bind to the receptor and engage a complex activation program that results in directed cell movement, release of inflammatory granule contents, and activation of a latent NADPH oxidase which is important for the production of metabolites of molecular oxygen. This pathway initiated by receptor-ligand interaction is critical in host protection from pyogenic infections. Similar signal transduction occurs in response to the inflammatory peptides C5a and IL-8.

Two other formyl peptide receptor like (FPRL) genes have been cloned based on their ability to hybridize to a fragment of the NFPR cDNA coding sequence. These have been named FPRL1 (Murphy et al. (1992) J. Biol Chem. 267:7637-7643) and FPRL2 (Ye et al. (1992) Biochem Biophys Res. Comm. 184:582-589). FPRL2 was found to mediate calcium mobilization in mouse fibroblasts transfected with the gene and exposed to formyl peptide. In contrast, although FPRL1 was found to be 69% identical in amino acid sequence to NFPR, it did not bind prototype N-formyl peptides ligands when expressed in heterologous cell types. This lead to the hypothesis of the existence of an as yet unidentified ligand for the FPRL1 orphan receptor (Murphy et al. supra).

### EXAMPLES

The example described below are for illustrative purpose only, and should not be construed to be limiting in any respect.

G-protein-coupled receptors (GPCRs) are involved in cell communication processes and with mediating such senses as vision, smell, taste, and pain. They constitute a prominent superfamily of drug targets, but an atomic-level structure is available for only one GPCR, bovine rhodopsin, making it difficult to use structure-based methods to design receptor-specific drugs.

There have been attempts to model the structure of GPCRs using homology modeling methods with either the bacteriorhodopsin or bovine rhodopsin crystal structure as template (Strader et al., 1994). Since there is only one known structure, these homology applications lead to transmembrane regions very similar to the bovine rhodopsin template structure. Moreover, many important GPCR targets have only low homology to bovine rhodopsin, making the models particularly unreliable (Archer et al., 2003). Thus the sequence identity of bovine rhodopsin to dopamine D2 receptor is 17%, to serotonin H1A 14%, and to G2A 13%, whereas good structures from homology models generally require >45% sequence identity.

GPCR structures have also been modeled using the properties of conserved residues in multiple sequence alignments followed by optimization of the structure using distance restraint to maximize the hydrogen bonds (Lomize et al., 1999). Distance restraints from various experiments were also used to predict the structure of bacteriorhodopsin (Herzyk and Hubbard, 1995). Comparing the TM helical region of their predicted structure to a bundle of ideal helices (i.e., not bent) superimposed on the bacteriorhodopsin electron cryomicroscopy structure, they reported a CRMS of 1.87 Å in the C-alphas.

Shacham et al. (2001) claim to have predicted the structure of bovine rhodopsin using an approach based on specificity of protein-protein interaction and protein-membrane interaction and the amphipathic nature of the helices. However, they have not yet provided any details of their method or of predictions on other GPCRs.

We have developed the MembStruk first principles computational method for predicting the three-dimensional structure of GPCRs. Here, we validate the MembStruk procedure by comparing its predictions with the high-resolution crystal structure of bovine rhodopsin. The crystal structure of bovine rhodopsin has the second extracellular (EC-II) loop closed over the transmembrane regions by making a disulfide linkage between Cys-110 and Cys-187, but we speculate that opening this loop may play a role in the activation process of the receptor through the cysteine linkage with helix 3. Consequently we predicted two structures for bovine rhodopsin from the primary sequence (with no input from the crystal structure)-one with the EC-II loop closed as in the crystal structure, and the other with the EC-II loop open. The MembStruk-predicted structure of bovine rhodopsin with the closed EC-II loop deviates from the crystal by 2.84 Å coordinate root mean-square (CRMS) in the transmembrane region main-chain atoms. The predicted three-dimensional structures for other GPCRs can be validated only by predicting binding sites and energies for various ligands. For such predictions we developed the HierDock first principles computational method. We validate HierDock by predicting the binding site of 11-cis-retinal in the crystal structure of bovine rhodopsin. Scanning the whole protein without using any prior knowledge of the binding site, we find that the best scoring conformation in rhodopsin is 1.1 Å CRMS from the crystal structure for the ligand atoms. This predicted conformation has the carbonyl 0 only 2.82 Å from the N of Lys-296. Making this Schiff base bond and minimizing leads to a final conformation only 0.62 Å CRMS from the crystal structure. We also used HierDock to predict the binding site of 11-cis-retinal in the MembStruk-predicted structure of bovine rhodopsin (closed loop). Scanning the whole protein structure leads to a structure in which the carbonyl 0 is only 2.85 Å from the N of Lys-296. Making this Schiff base bond and minimizing leads to a final conformation only 2.92 Å CRMS from the crystal structure. The good agreement of the ab initio-predicted protein structures and ligand binding site with experiment validates the use of the MembStruk and HierDock first principles' methods. Since these methods are generic and applicable to any GPCR, they should be useful in predicting the structures of other GPCRs and the binding site of ligands to these proteins.

### Example 1

Validation of the Force Fields

The crystal structure of bovine rhodopsin (resolution, 2.80 Å) was downloaded from the protein structure database (PDB entry 1F88). The Hg ions, sugars, and waters were deleted from this structure. This crystal structure is missing 10 complete residues in loop regions and the side-chain atoms for 15 additional residues. We added the missing residues and side chains using WHATIF (Vriend, 1990). Then we added hydrogens to all the residues using the PolyGraf software. We then fixed the TM helices and minimized (using conjugate gradients) the structure of the loop region to a root mean-square force of 0.1 kcal/mol per Å. The potential energy of the entire structure of rhodopsin was then minimized (using conjugate gradients) to a root mean-square force of 0.1 kcal/mol per Å. This minimized structure deviates from the x-ray crystal structure by 0.29 Å coordinate root mean-square (CRMS) error over all atoms in the crystal structure. This is within the resolution of the crystal structure, thus validating the accuracy of the FF and the charges. This FF-minimized crystal structure is denoted as Ret(x)/closed(xray).

### Example 2

Various Bovine Rhodopsin Structures (Crystal or Predicted)

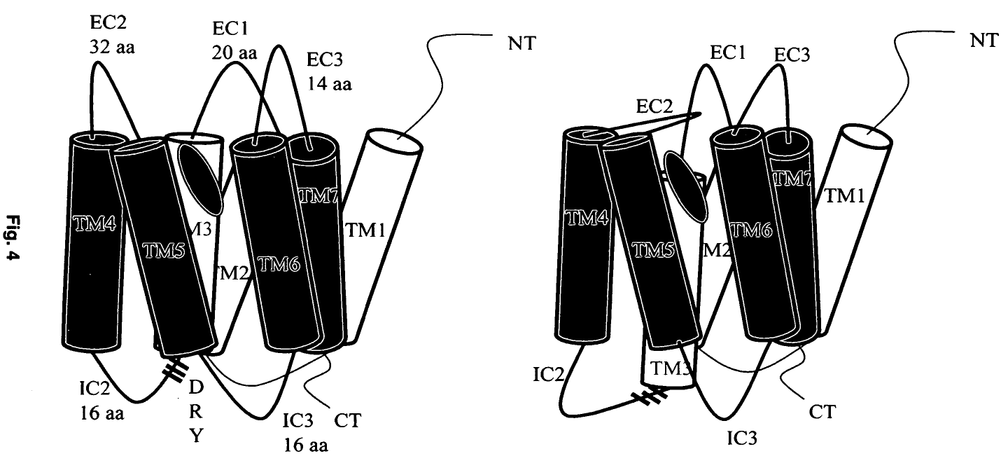

The crystal structure for the retinal/rhodopsin complex has a well-defined β-sheet structure for EC-II, which might be involved as a mobile gate for entry of 11-cis-retinal on the extracellular side of rhodopsin. Such a gating mechanism is illustrated in FIG. 4, in which the helix 3 coupled to this loop by a cysteine bond is the gatekeeper which responds to signaling structural substrates of rhodopsin as follows:

When rhodopsin binds 11-cis-retinal, the ground state conformation of the receptor is stabilized, thus shifting helix 3 toward the intracellular side (forming the D(E)RY-associated salt bridges at that end) and closing the EC-II loop. In fact, 11-cis-retinal has been shown to be an inverse agonist for G-protein signaling (Okada et al., 2001).

In response to absorption of a photon, the 11-cis-retinal isomerizes to the all-trans conformation, inducing helix 3 to shift toward the extracellular side. This induction of helix 3 movement may be direct or indirect. It may be due to a direct clash of helix 3 with all-trans-retinal. This is consistent with the result of a cross-linking experiment in which the ionone ring of retinal interacts with Ala-269 when the receptor is activated (Borhan et al., 2000). This may occur because the trans-retinal clashes with helix 3 of the ground state rhodopsin crystal structure (Bourne and Meng, 2000). The induction of helix 3 movement may also occur indirectly in the following way: 11-cis-retinal as observed in the crystal structure interacts with aromatic side chains Trp-265 and Tyr-268 on helix 6. But all-trans-retinal does not have this stabilizing interaction with helix 6, which should decrease the energy barrier for helix 6 rotation (this has been observed in preliminary MD calculation we carried out, and in reports in the literature; Saam et al., 2002).

This motion (of helix 3 or helix 6) breaks the DRY-associated salt bridges (Greasley et al., 2002) at the intracellular side. Helix 3 may have fewer constraints to movement, but since it is coupled by a disulfide linkage to the EC-II loop, movement on helix 3 would likely cause an opening of the EC-II loop to allow Schiff base reversion and exit of the free all-trans-retinal ligand. The breaking of this DRY salt bridge would also allow hinge motion (Altenbach et al., 2001a,b) of helix 6 to expand the molecular surface at the cytoplasmic end for G-protein binding. This model is consistent with the experimental mutations studies in which the disulfide has been shown to be important for ligand binding and receptor activation (Schöneberg et al., 2002).

Building the loops without the constraint of coupling these cysteines leads to an open EC-II loop very different from the crystal structure of bovine rhodopsin. It is likely that both the open loop and closed loop structures play an important role in GPCRs, and indeed general observations of GPCRs suggests two distinct forms, one of which leads to activation of G-protein and one of which does not. We consider that one of these is likely the closed form and the other the open form. It seems likely that the ligand might not be able to diffuse into the active site when the loop is closed, and hence for most GPCRs (other than bovine rhodopsin) we visualize the process of activation as 1), the GPCR with the open form of EC-II loop can bind selectively to the appropriate ligand; 2), binding of the ligand favors closing of the EC-II loop; and 3), after closure of the loop, G-protein activation may begin.

Thus we have built two structures for bovine rhodopsin (here, MS denotes that the structure was predicted using MembStruk): Apo/closed(MS) has the cysteine coupling observed in the crystal and is the structure we compare to experiment after binding the retinal; and Apo/open(MS) is built without a constraint, forming what we believe would be the configuration which binds initially to the ligand.

Several bovine rhodopsin structures were generated based on its solved crystal structure and energy minimization using the DREIDING FF. These structures are briefly described below.

Ret(xtal)/closed(xtal) was obtained from the crystal structure of bovine rhodopsin by minimizing using the DREIDING FF. It deviates from the crystal structure by 0.29 Å CRMS. It has retinal bound as in the crystal structure and has the closed form of the EC-II loop. The retinal conformations differ by 0.22 Å CRMS. This further validates the FF. Since they differ so little, the retinal in the nonminimized crystal structure, Ret(xtal-noFF), is used as the reference structure for the HierDock validation step.

Apo/closed(xtal) was obtained from Ret(xtal)/closed(xtal) by removing the retinal and adding the proton to Lys-296. It was minimized without ligand. It deviates from the crystal structure by 0.74 Å CRMS. It is likely that this is a lower bound on the change in structure upon removal of the retinal. For a more complete optimization, MD could be used.

Ret(HD)/closed(xtal) is the predicted structure for 11-cis-retinal obtained by applying HierDock to Apo/closed(xtal) and then forming the Schiff base linkage to Lys-296 and minimizing. The Ret(HD) deviates from Ret(xtal) by 0.62 Å CRMS. To distinguish the error in ligand conformation due to the HierDock procedure from that due to MembStruk, the structure Ret(HD)/closed(xtal) will serve as the reference structure to compare to the predicted ligand conformations in the MembStruk structures.

Apo/closed(MS) is the MembStruk-predicted structure of the closed form, without the retinal. The TM bundle for this structure deviates by 2.84 Å CRMS main-chain atoms from Apo/closed(xtal) (4.04 Å CRMS for all TM atoms, excluding H).

Ret(HD)/closed(MS) is the predicted structure for 11-cis-retinal in the Apo/closed(MS) rhodopsin structure, obtained by applying HierDock to Apo/closed(MS) and then forming the Schiff base linkage to Lys-296 and minimizing the energy. The Ret(HD) deviates from Ret(HD)/closed(xtal) by 2.92 Å CRMS.

Apo/open(MS) is the MembStruk-predicted structure of bovine rhodopsin without the retinal. There are no experiments with which to compare. This structure differs in the TM region from Apo/closed(MS) by 0.11 Å.

Ret(HD)/open(MS) is the predicted structure for 11-cis-retinal in rhodopsin obtained by applying HierDock to Apo/open(MS) and then forming the Schiff base linkage to Lys-296 and minimizing. There are no experiments with which to compare. The retinal differs from that in Ret(HD)/closed(MS) by 1.74 Å.

### Example 3

Validation of the HierDock Protocol on the Crystal Structure of Bovine Rhodopsin

Bovine rhodopsin (a member of the opsin family) is the only GPCR to be crystallized in its entirety at a high resolution (2.8 A). Thus we used this system as a test to validate the HierDock protocol for predicting the binding sites of GPCRs.

To test HierDock, we used the Apo/closed(xtal) structure with the retinal removed and minimized. First we did a complete HierDock scan as outlined above to predict the binding of 11-cis-retinal to bovine rhodopsin. The crystal structure of rhodopsin has the 11-cis-retinal covalently bound to Lys-296 (between the aldehyde of 11-cis-retinal and the N of the Lys), but for docking we cannot have a covalent bond to the crystal. Thus we docked the full 11-cis-retinal ligand (containing a full aldehyde group) and considered the Lys-296 to be protonated.

We applied Steps 1-2 of the HierDock described above for all 13 overlapping regions for Step 2 shown in FIG. 5. The initial scan of the entire rhodopsin (Steps 1 and 2 in Function Prediction for GPCRs) gave two good binding regions shown as the red boxes in FIG. 5. The data for this scanning step are shown in Table 2.

TABLE 2

| Box | Top 5% after coarse-grain ranking |
|---|---|
| 1 | 2596, 2941, 2991, 3011, 4281 |
| 2 | 4440, 4621, 4625, 5509, 5513 |
| 3 | 2338, 2375, 2409, 2566, 2571 |
| 4 | 5844, 5961, 6006, 6244, 6278 |
| 5 | None passed buried surface criteria |
| 6 | 102, 118, 131, 136, 208 |
| 7 | 1366, 1370, 1374, 1374, 1379 |
| 8 | No conformations generated from DOCK |
| 9 | 12026 |
| 10 | 82, 139, 153, 377, 380 |
| 11 | 2348, 2348, 2566, 2843, 2843 |
| 12 | No conformations generated from DOCK |
| 13 | 551, 734, 931, 1110, 1226 |

Results from the coarse-grain docking step of HierDock to predict the binding site(s) in Apo/closed(xray). The energies of the top 5% after ranking (level 2 of HierDock) are shown for each box. Among all boxes, the best coarse-grain score is underlined. The scores within 100 kcal/mol of the top score are shown in bold.

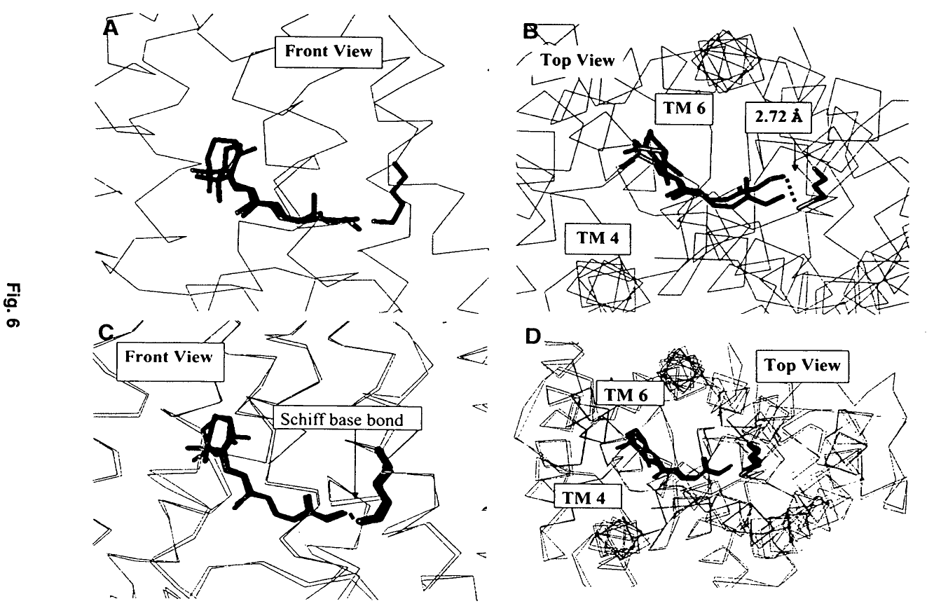

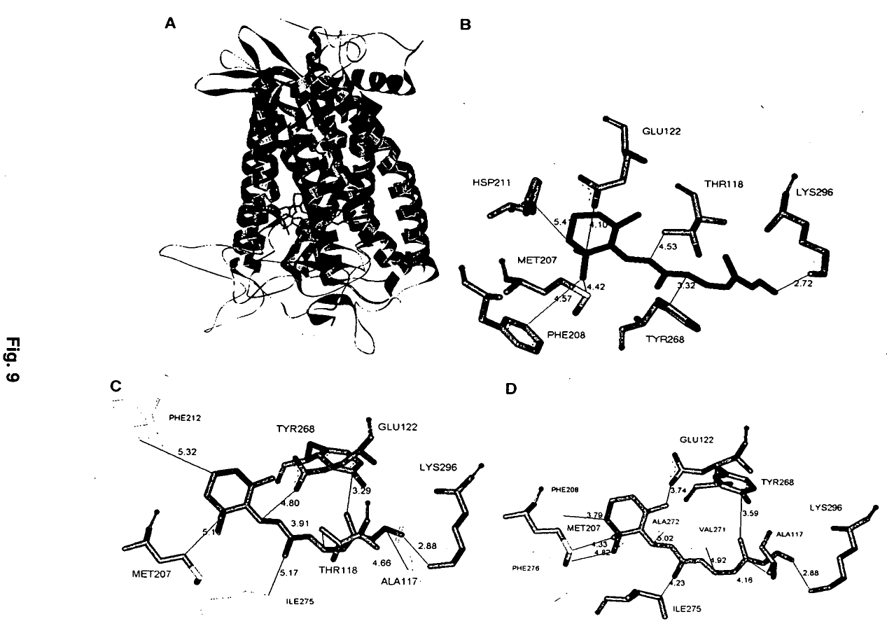

The final optimized best binding structure for the retinal/rhodopsin complex from Step 6 of HierDock deviated by 1.11 Å CRMS from the ligand in the crystal structure as seen in FIG. 6, A and B. The binding site (defined as the seven residues that contribute at least 1 kcal/mol to the bonding) of this ligand is shown in FIG. 9 B. Lys-296 has hydrophilic interactions whereas the other side chains have van der Waals interactions. This docked structure has the retinal O2.72 Å from the N of Lys-296. In addition, the retinal O and the closest H of the protonated Lys-296 N are just 2.35 Å apart, close enough to form an H-bond (likely an intermediate step before Schiff base formation). We then coupled these two units to form the covalent CN bond to Lys-296 while eliminating the H₂O. After minimizing the full ligand-protein structure, we find that the predicted structure for 11-cis-retinal bonded to the protein deviates from the crystal structure by only 0.62 Å CRMS as shown in FIG. 6, C and D. Most of this discrepancy results because the FF-minimized structure of the retinal has the ionone ring in a chair conformation which was retained in our docking procedure, whereas the crystal structure has the ionone ring in a half-chair conformation (which we calculate to be 2 kcal/mol higher in energy than the chair conformation within the minimized complex). This retinal/protein complex minimized with the DREIDING FF, denoted Ret(HD)/closed(xtal), serves as the reference structure for comparing the predicted structures in later sections. We consider that these results validate the HierDock protocol for a GPCR.

In addition, we used HierDock to determine the binding site and best scoring ligand conformation for all-trans-retinal, with the binding energy calculated using Eq. 1 above. The binding energy for 11-cis-retinal was -1 kcal/mol whereas that for all-trans-retinal was 31 kcal/mol, a difference of 32 kcal/mol. This compares well with the experimental result that the retinal ligand/protein complex stores 34.7±2.2 kcal/mol upon isomerization in the protein (Okada et al., 2001). This stored energy might be used to induce rigid-body helical motions needed for receptor activation and G-protein binding.

### Example 4

Validation of the Method by Predicting TM Helices in Bovine Rhodopsin

We have used the TM2ndS algorithm for predicting the structure for about 10 very different GPCR classes (Vaidehi, N., W. B. Floriano, R. Trabanino, S. E. Hall, P. Freddolino, E. J. Choi, G. Zamanakos, and W. A. Goddard. 2002. Prediction of structure and function of G-protein-coupled receptors. Proc. Natl. Acad. Sci. USA. 99:12622-12627). In each case the predicted binding site and binding energy agrees well with available experimental data, providing some validation of the TM region prediction. However only for bovine rhodopsin can we make precise comparisons to experimental structures. We have also used TM2ndS to predict the TM regions and length of TM regions for membrane proteins for which crystal structure is available. Our predictions give 100% of the database as membrane proteins. The length of the TM regions is predicted to within 2 residues of the crystal structure on either carboxy or amino terminus.

The crystal structure of bovine rhodopsin (resolution, 2.80 A) was downloaded from the protein structure database (PDB entry 1F88). The Hg ions, sugars, and waters were deleted from this structure. This crystal structure is missing 10 complete residues in loop regions and the side-chain atoms for 15 additional residues. We added the missing residues and side chains using WHATIF (Vriend, 1990, incorporated herein by reference). Then we added hydrogens to all the residues using the PolyGraf software.

FIG. 2 compares the predictions of TM helical regions for bovine rhodopsin to the TM helical regions as defined in the crystal structure paper (Palczewski, K., Kumasaka, T., Hori, T., Behnke, C., Motoshima, H., Fox, B., Trong, I., Teller, D., Okada, T., Stenkamp, R., Yamamoto, M., Miyano, M., (2000), Science, 289, 739-745). Upon conducting an analysis of the phi-psi angles using PROCHECK (Laskowski, R. A., MacArthur, M. W., Moss, D. S., Thornton, J. M., 1993, J. Appl. Cryst., 26,283-291), we found a slightly different helical assignment. Here we consider the experimental structure to be in an α-helix if −37<φ<−77 and −27<Ψ<−67. Those residues which fall outside this range are indicated in lowercase letters. The largest error in TM helical prediction is 5 residues at the C-terminal end of helix 5. This error may be corrected by an improved capping procedure in which residues near the ends of predictions are checked to be helix breakers and broken at those positions if the helix length remains above the 21 residue lower limit. The next largest error is 3 residues in the N-terminal end of helix 6. The rest of the errors are less than 3 residues. Thus, the predictions are found to agree well with the crystal structure. Detailed analysis of the prediction results are provided below.

For TM1, the TM2ndS prediction adds P at the start and H at the end. In a subsequent 3-D protein structure prediction using MembStruk3.5 (see U.S. Ser. No. 60/494,971, supra), the final structure ((φ, Ψ) for this P is (not-applicable [N-terminus], −43.6) and for this H is (−54.3, −32.4), whereas the values obtained in the crystal structure are (−44.3, −24.9) and (−72.5, 69.5), respectively. Since P and possibly H might be expected to break the helix, it is possible to modify the procedure to exclude such terminal P or H in the helix. In that case, the prediction would be exactly the same as the experimental data.

For TM2, the TM2ndS prediction adds HG at the end. In our final structure, the ((φ, Ψ) for this H and G are (−73.6, −80.9) and (−55.0, 148.8), whereas the values obtained in the crystal structure are (−74.2, 0.5) and (66.1, 9.0), respectively. The crystal structure article considered the H as part of the helix. Since HG could be expected to break the helix, it is possible to modify the procedure to exclude the terminal HG in the helix. In fact, the HG angles in our final structure fall outside our criteria for α-helicity as a result of the MembStruk optimization of the structure.

For TM3, the TM2ndS prediction misses the RYVVV (SEQ ID NO: 22) assigned in the crystal structure to the helix. Since the first and second V do not have ((φ, Ψ) in the usual range for α-helices, we consider that the VVV should be excluded. However, the polar character of RY leads TM2ndS to miss assigning them as part of the helix. The crystallographic ((φ, Ψ) values for R and Y residues are (−55.5, −63.8) and (−44.6, −56.3), whereas the values obtained in our final structure are (76.7, −51.4) and (−62.9, 119.2). It should be pointed out that the B-factors on the cytoplasmic end of the rhodopsin crystal structure are high in this region of the helix (PDB entry 1F88). This indicates that the helix is probably fluxional even when the receptor is not activated. Consequently caution should be used when comparing our predictions with the crystal structure at this end. Also, because the helices are translated to align hydrophobic centers in a later step of the procedure, this uncertainty in TM helical prediction may only lead to local errors in atomic structure.

For TM4, the TM2ndS prediction adds G at the end and misses N at the start. The crystallographic ((φ, Ψ) for these N and G residues are (−43.5, −59.6) and (169.8, 5.4), whereas the values obtained in our final structure are (−93.9, 119.6) and (112.5, −18.4). Thus the predictions are fine even though the G and N were mis-assigned. It is possible to modify the procedure to exclude a terminal G.

Compared to the crystal structure assignment, the TM2ndS prediction for TM5 adds LVF at the end and misses N at the start. In addition the GQ at the end terminus in the crystal structure assignment have ((φ, Ψ) outside the range for α-helices. Thus the terminal GQLVF (SEQ ID NO: 23) in the TM2ndS predictions may be in error, the largest error of any of the predictions. The crystallographic ((φ, Ψ)) for these N and LVF residues are (31 69.3, −51.1), (−48.2, −36.7), (−39.6, −27.1), and (−58.0, −26.5), whereas the values obtained in our final structure are (−109.9, −162.4), (−55.1, −47.8), (−63.4, −59.0), and (−81.5, 59.3). The rhodopsin crystal structure has high B-factors for the intracellular end of TM5 (just as for helix 3), suggesting caution in making comparisons.

For TM6, the TM2ndS prediction adds H at the end and misses EVT at the start. The crystallographic ((φ, Ψ) values for these EVT and H residues are (−57.6, −53.0), (−54.1, −55.7), (−56.3, −52.3), and (−81.3, 48.8), whereas the values obtained in our final structure are (−74.4, −72.3), (−73.1, 130.8), (−16.9, −53.0), and (7.1, 87.7). Thus the predictions are fine despite the misassignments. We are considering modifying our procedure to exclude a terminal H. In fact, the H angles in our final structure fall outside our criteria for α-helicity as a result of the MembStruk optimization of the structure.

For TM7, the TM2ndS prediction adds P at the start and misses Y at the end. The crystallographic ((φ, Ψ) for the P and Y residues are (−30.2, −48.1) and (−46.0, −55.0), whereas the value for P obtained in our final structure is (−43.6, −23.2). Since the current MembStruk protocol does not model the structures of the C- and N-termini, we did include the Y in our structure. Thus the predictions are fine despite the misassignments. It is possible to modify the procedure to exclude a terminal P, but it is not obvious that a modified method would automatically include the Y. In fact, the P angles in the final 3-D MembStruk predicted structure fall outside our criteria for α-helicity as a result of the MembStruk optimization of the structure.

Overall, we consider that the predictions agree sufficiently well with the crystal structure to be useful in building them into the assembly. In addition, we can see several improvements in the capping procedure of TM2ndS that could have decreased the errors in predicting which residues near the ends are considered to be helix breakers for capping the TM helices. However, this article is meant to validate the procedure we have been applying to many systems. Thus, the overall procedure may be kept without modification on the basis of our only independent validation.

### Example 5

Structure and Function Prediction for Bovine Rhodopsin

We used MembStruk3.5 to perform structure and function prediction for bovine rhodopsin, using only the protein sequence.

For the apo-rhodopsin we predicted two structures, one with the open EC-II loop and one with closed EC-II loop. These represent two different states of rhodopsin likely to play a role in activation of G-protein. The crystal structure of rhodopsin has a closed EC-II loop with the 11-cis-retinal bound to it. To validate this predicted structure, we should compare to the crystal structure for apo-rhodopsin (without a bound 11-cis-retinal). However, this crystal structure for the apo protein is not available. Thus instead we will compare the predicted structure to the minimized crystal structure of bovine rhodopsin after removing the 11-cis-retinal. In making these comparisons, we predicted two structures for apo-rhodopsin: 1), the open form, where no restrictions were made on the structure of EC-II loop, i.e., Apo/open(MS); and 2), the closed form, where we assumed that EC-II makes the same cysteine linkage as observed in the crystal structure, i.e., Apo/closed(MS).

The predicted TM domains are compared to the rhodopsin crystal structure in FIG. 2 and discussed in Step 1, Helix Capping in TM2ndS.

After optimization of the helices using MD (300 K for 500 ps), most helices yield the same bends as in the crystal. Thus helices 2 and 6 undergo significant bending (due to Pro-267 in helix 6 and due to Gly-89 and Gly-90 in helix 2), which is consistent with spin-labeling electron paramagnetic resonance experiments (Farrens et al., 1996). In addition, we find that helix 7 bends near the two prolines, which has also been shown by spin-labeling experiments (Altenbach et al., 2001a,b). We find that helix 1 undergoes significant bending due to a Gly/Pro combination, but this has not yet been studied experimentally. Such bending at hinge sites may be important for expanding the molecular surface needed at the cytoplasmic side to allow G-protein binding. We find similar hinge-bending with MD when the trans-isomer is bound to the helix assembly.

After assembling the optimized helices again into a bundle, we carried out RotMin on helices 3 and 5, the only helix pair with a potential salt bridge. The resulting seven-helix bundle was then inserted into a lipid bilayer, and optimized using rigid-body molecular dynamics as described in Step 4 of The MembStruk Protocol for Predicting Structure of GPCRs. This step leads to optimization of the vertical helical positions, relative helical angles, and rotations of the individual helices within a lipid environment. The CRMS difference before and after this rigid body MD is 1.10 Å for all atoms and 0.98 Å for main-chain atoms. This is consistent with the changes during this optimization step for other GPCRs (Vaidehi et al., 2002).

After adding the intracellular and extracellular loops, optimizing the side chains, and then optimizing the structure in vacuum with the TM helical region fixed (to eliminate bad contacts in the loop region), we then optimized the entire structure allowing all bonds and angles to change. These ab initio predictions of the structure were carried out for both the open and closed forms of the EC-II loop in apo-rhodopsin leading to the Apo/open(MS) and Apo/closed(MS) structures, where MS denotes a MembStruk-derived structure, and open or closed denotes the open or closed form of the EC-II loop. Although the crystal structure has the 11-cis-retinal bound, we will compare the predicted apo-rhodopsin structures to the minimized apo-protein of the crystal structure, Apo/closed(xray).

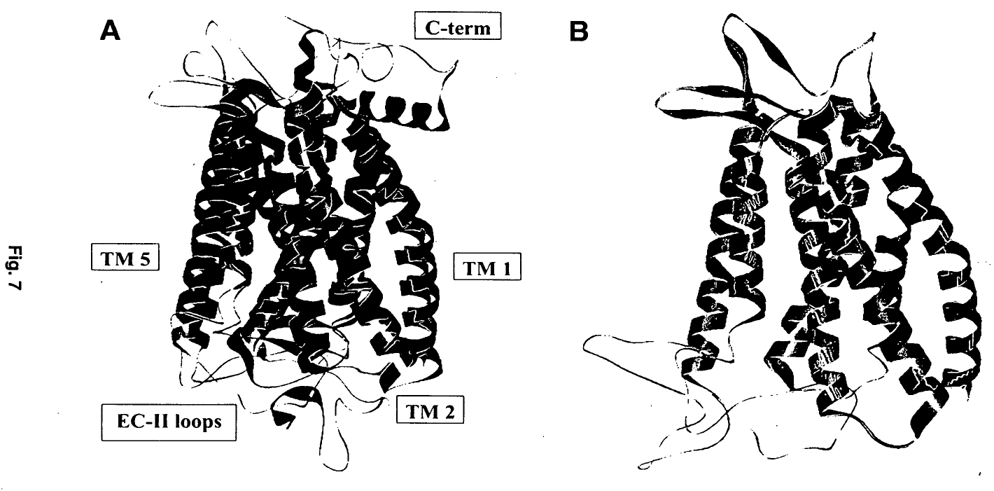

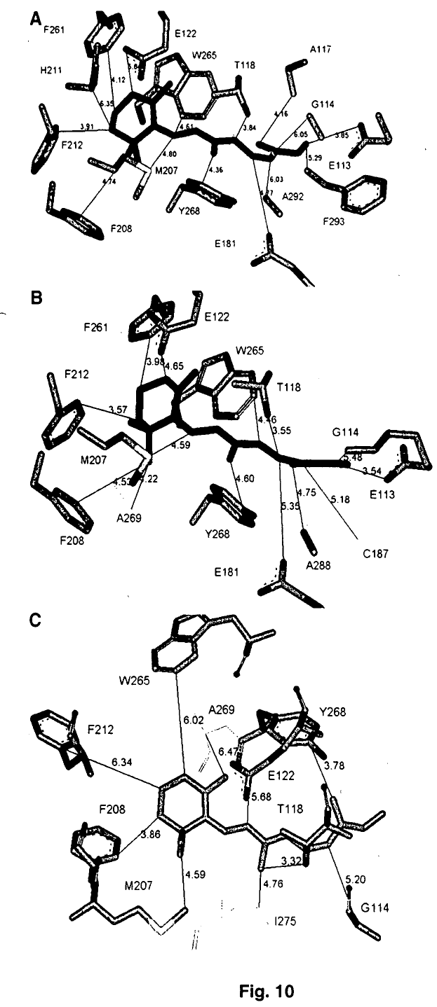

Comparing Apo/closed(MS) to Apo/closed(xray), we find a CRMS difference of 2.85 Å in the main-chain atoms and 4.04 Å for all the atoms in the TM helical region. These structures are compared graphically in FIG. 7A. Comparing all residues including loops (but ignoring the residues not present or complete in the x-ray structure), the predicted structure differs from the crystal structure by 6.80 Å in the main chain and 7.80 Å CRMS for all atoms. The major contribution to this CRMS is the low-resolution loop region, which is likely to be quite fluxional and may be very different between crystal and solution. Specifically, the predicted topology and φ-Ψ angles of the EC-II loop are consistent with that of a B-sheet. However, the specific twist of this β-sheet in the x-ray structure was not predicted well. Although this may be partly due to packing effects in the crystal structure, we consider that our prediction of the general topology of the EC-II loop to act as a “plug” to restrict retinal binding is adequate but that specific interactions with retinal may not be predicted well. In the function prediction results discussed below in the subsection called Apo/Closed(MS), we find that there are no specific favorable interactions between the ligand and the EC-II loop before Schiff base bond formation in the crystal structure (FIG. 9 B). Thus the EC-II may function initially primarily as an unspecific “plug” to disfavor certain ligand conformations. After Schiff base bond formation, the ligand is then stabilized by Glu-181 in the EC-II loop (FIG. 10A).

We find that Apo/open(MS) deviates from Apo/closed(MS) by a CRMS difference of 0.11 Å in the main-chain atoms and 0.68 Å for all the atoms in the TM helical region. These structures are compared graphically in FIG. 7 C. This small difference in CRMS in the transmembrane region suggests that we need to carry out long timescale molecular dynamics for the helices to accommodate the EC-II loop conformational change. Comparing all residues, the predicted structure differs from the crystal structure by 4.74 Å in the main chain and 5.0 Å CRMS for all atoms. There is no experimental structure Apo/open(xray) with which to compare Apo/open(MS).

### Example 6

HierDock Function Prediction for Apo-rhod (MS) Structures

Except for bovine rhodopsin, essentially all applications of HierDock to GPCRs must use predicted structures rather than experimental structures. Given the deviations in predicting the GPCR structure (2.8 Å CRMS in the TM helical region), it is important to know if it is possible to get accurate predictions in the binding site and binding energy. For this purpose, we tested how well HierDock determines the binding site of 11-cis-retinal to the predicted rhodopsin structures Apo/open(MS) and Apo/closed(MS).

Here we repeated the full process described in Function Prediction for GPCRs. The void space for both the Apo/open(MS) and Apo/closed(MS) structures were partitioned into fourteen 7 Å×7 Å×7 Å boxes and scanned for the putative binding site of 11-cis-retinal (using the same ab initio FF-optimized ligand structure as in Validation for Function Prediction HierDock Protocol for 11-cis-retinal on Bovine Rhodopsin). Again the molecule includes the aldehyde group (no assumed formation of the Schiff base).

Apo/closed(MS)

Scanning the entire Apo/closed(MS) receptor to find the binding site and binding energy for 11-cis-retinal used the steps described in Computational Methods. The best scoring conformation for 11-cis-retinal and its associated binding site, denoted as NoSB-Ret(HD)/closed(MS), are shown in FIG. 9C. Here NoSB indicates the structure without the Schiff base covalent bond between the aldehyde group of 11-cis-retinal and Lys-296. This conformation (no covalent attachment) differs from Ret(HD)/closed(xtal) by 3.2 Å CRMS. We should emphasize that the Apo/closed(MS) structure was constructed purely from ab initio predictions with MembStruk, with no input from the x-ray crystal structure. Thus nowhere did we assume a lysine covalent bond with retinal in any of the docking procedures. Yet, the predicted structure identifies which Lys can bond to the retinal, with 2.85 Å between the predicted position of the retinal oxygen and the predicted position of the Lys-296 nitrogen.

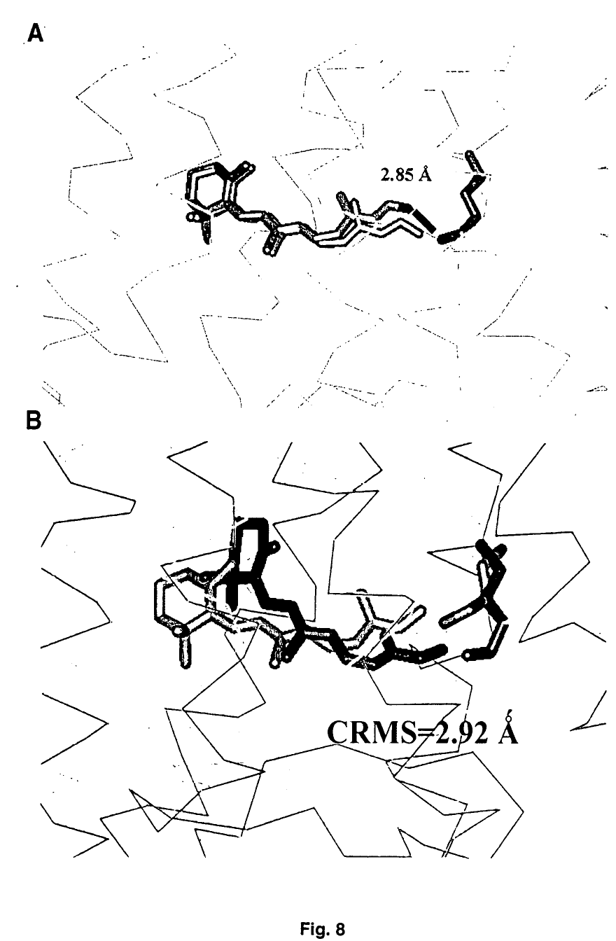

Then starting with NoSB-Ret(HD)/closed(MS), we formed this Schiff s base bond (eliminating H₂0), and optimized the full ligand-protein complex with conjugate gradient minimization to obtain the Ret(HD)/closed(MS) structure. This differs from Ret(HD)/closed(xtal) by 2.92 Å CRMS. These structures are compared in FIG. 8, A and B.

A second criterion for validity of the predicted binding site is to identify the residues interacting most strongly with the ligand, which can be used to predict mutational studies for validation and to design antagonists or agonists. Considering the binding site to be all residues within 5.0 Å of the ligand leads to 30 residues for Ret(xtal)/closed(xtal). For Ret(HD)/closed(MS) we find 26 residues (26 in common with Ret(x)/closed(xtal)) and for Ret(HD)/closed(xtal) we find 23 residues (15 in common with Ret(x)/closed(xtal)) in the binding site. More important is to establish which of these residues is responsible for ligand stabilization. Thus we calculated the interactions of all amino acid residues within 5 Å of the ligand and kept those that have a more favorable interaction than −1 kcal/mol interaction energy with the ligand. For Ret(xtal)/closed(xtal) this leads to the 15 residues shown in FIG. 10A. For Ret(HD)/closed(MS) we find 10 residues (8 in common with Ret(x)/closed(xtal)) shown in FIG. 10B and for Ret(HD)/closed(xtal) we find 14 residues (12 of which in common with Ret(x)/closed(xtal)) shown in FIG. 10 C. The interaction energies of the residues are shown in Table S2. The side chains identified as important include Trp-265 and Tyr-268, which have been implicated (Lin and Sakmar, 1996) to modulate the absorption frequency of 11-cis-retinal.

TABLE S2
Residues within a 5 Å shell (of retinal with Schiff base bond formed)
which interact more favorably than an interaction energy of −1 kcal/mol
with the ligand are shown in order of decreasing interaction.
The interaction energy values are shown in parentheses.

| Ret(xtal)/closed(xtal) | Ret(HD)/closed(xtal) | Ret(HD)/closed(MS) |
|---|---|---|
| Glu122 (−21.9) | Glu122 (−19.8) | Glu122 (−18.2) |
| Glu113 (−20.3) | Glu113 (−18.0) | Thr118 (−4.0) |
| Glu181 (−18.0) | Glu181 (−14.9) | Tyr268 (−4.0) |
| Trp265 (−6.5) | Trp265 (−6.8) | Phe208 (−3.2) |
| Tyr268 (−4.8) | Thr118 (−5.2) | Met207 (−1.2) |
| Thr118 (−3.9) | Tyr268 (−5.2) | Trp265 (−1.1) |
| Ala292 (−2.9) | Met207 (−2.7) | Gly114 (−1.1) |
| Ala117 (−2.3) | Ala292 (−2.3) | Ala269 (−1.1) |
| Phe208 (−2.0) | Phe212 (−1.9) | Phe212 (−1.0) |
| His211 (−1.8) | Phe208 (−1.8) | Ile275 (−1.0) |
| Phe212 (−1.7) | Gly114 (−1.8) | |
| Gly114 (−1.6) | Cys187 (−1.3) | |
| Met207 (−1.6) | Phe261 (−1.2) | |
| Phe261 (−1.4) | Ala269 (−1.2) | |
| Phe293 (−1.1) | | |

To provide an idea of how the retinal binds before Schiff base bond formation, we also considered the binding site as all residues within 5.0 Å of the ligand before bond formation that have a more favorable interaction than −1 kcal/mol interaction energy with the ligand. For NoSB-Ret(HD)/closed(xtal) this leads to the seven residues shown in FIG. 9 B. For NoSB-Ret(HD)/closed(MS) we find eight (six in common with NoSB-Ret(HD)/closed(xtal)) shown in FIG. 9C. The interaction energies of the residues are shown in Table S1. Of the top interacting residues (three residues) in NoSB-Ret(HD)/closed(xtal), two (Tyr-268 and Lys-296) are also shown to rank among the top three in NoSB-Ret(HD)/closed(MS). The residue which was missed (Thr-118) ranked lower in NoSB-Ret(HD)/closed(MS) because it is actually closer to the retinal (in comparison with the NoSB-Ret(HD)/closed(xtal) structure), with distances as low as 2.8 Å (whereas an optimal van der Waals distance is 3.4 Å) to the polyene chain of retinal.

TABLE S1
Residues within a 5 Å shell (of retinal without Schiff base bond formed)
which interact more favorably than an interaction energy of −1 kcal/mol
with the ligand are shown in order of decreasing interaction.
The interaction energy values are shown in parentheses.

| NoSBRet(HD)/closed | NoSB-Ret(HD)/closed | |
|---|---|---|
| (xtal) | (MS) | NoSBRet(HD)/open(MS) |
| Thr118 (−4.6) | Lys296 (−9.6) | Lys296 (−7.3) |
| Tyr268 (−3.9) | Glu122 (−3.0) | Tyr268 (−3.4) |
| Lys296 (−2.6) | Tyr268 (−2.6) | Glu122 (−2.9) |
| Hsp211 (−1.8) | Ala117 (−1.9) | Ala272 (−2.7) |
| Glu122 (−1.4) | Thr118 (−1.9) | Ile275 (−2.5) |
| Phe208 (−1.4) | Ile275 (−1.6) | Ala117 (−2.2) |
| Met207 (−1.2) | Met207 (−1.1) | Val271 (−1.9) |
| | Phe212 (−1.0) | Met207 (−1.4) |
| | | Phe208 (−1.3) |
| | | Phe276 (−1.1) |

We conclude that the MembStruk-predicted structure is useful for predicting binding sites sufficiently well to direct mutation studies to elucidate the precise site.

Apo/Open(MS)

We scanned the entire Apo/open(MS) receptor to find the binding site and binding energy for 11-cis-retinal using the steps described in Computational Methods. The best scoring conformation for 11-cis-retinal and its associated binding site, denoted as NoSB-Ret(HD)/open(MS), are shown in FIG. 9D. The predicted structure identifies which Lys can bond to the retinal, with 2.87 Å between the predicted position of the retinal oxygen and the predicted position of the Lys-296 nitrogen.

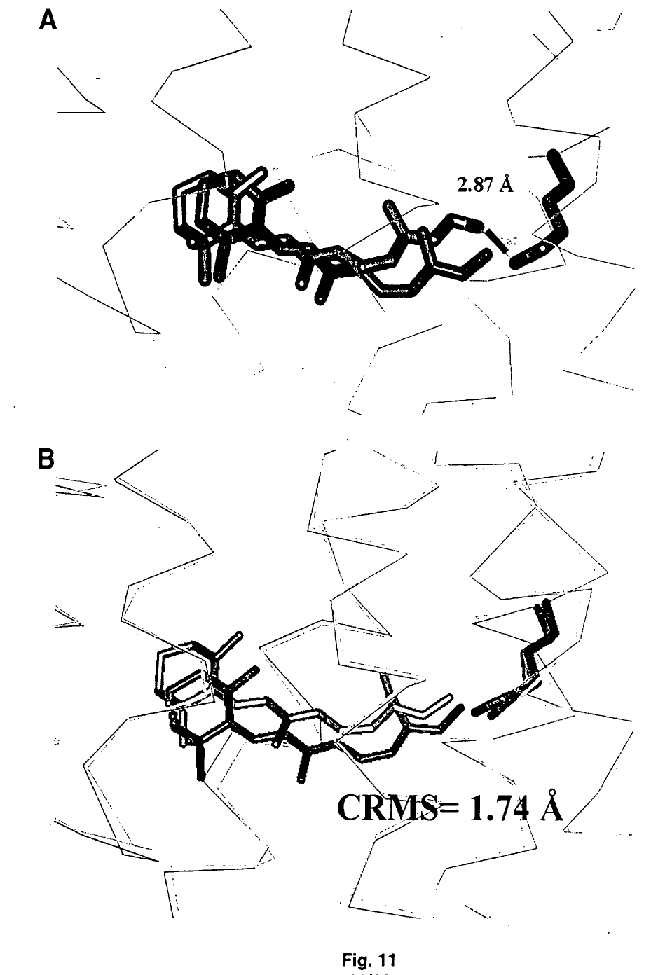

Then starting with NoSB-Ret(HD)/open(MS), we formed this Schiff base bond (eliminating H₂O), and optimized the full ligand-protein complex with conjugate gradient minimization to obtain the Ret(HD)/open(MS) structure, This is no experimental structure with which to compare, but this structure differs from Ret(HD)/closed(MS) by 1.7 Å CRMS. These structures are compared in FIG. 11, A and B.

A second criterion for validity of the predicted binding site is in identifying those residues close to the ligand to consider for mutational studies and drug design. Considering the binding site of NoSB-Ret(HD)/open(MS) as all residues within 5.0 Å of the ligand, the amino acid residues which interact with <−1 kcal/mol interaction energy with the ligand (10 residues) are shown in FIG. 9 D. Of these, six residues are also shown to interact with the ligand in the NoSB-Ret(HD)/closed(MS) structure discussed in the subsection called Apo/Closed(MS). We also find four additional residues (Phe-276, Phe-208, Val-27 1, and Ala-272) that do not bind with 1 kcal/mol in the NoSB-Ret(HD)/closed(MS) structure. This difference results from the shift in the retinal binding site upon opening of the EC-II loop. Thus, we consider that the retinal bound to the open-loop structure is partially stabilized by van der Waals interactions.

Using MembStruk we predicted the two structures Apo/open(MS) with the EC-II in an open conformation and Apo/closed(MS) with it closed. The crystal structure of rhodopsin has the closed configuration in which EC-II has a well-defined β-sheet structure with the 11-cis-retinal bound. We speculate that changes in the structure of this loop are involved in activation of G-protein and in the entry of 11-cis-retinal on the extracellular side of rhodopsin. The idea is illustrated in FIG. 4, in which the helix 3 coupled to this loop by a cysteine bond is the gatekeeper which responds to signaling structural substrates of rhodopsin as follows.

Starting with the inactive form Ret/closed with 11-cis-retinal covalently linked to the rhodopsin, the response to visible light causes the 11-cis-retinal to isomerize to all-trans-retinal, which in turn causes changes in the conformation (Altenbach et al., 2001a,b, 1996; Farrens et al., 1996) near the retinal that eventually leads to a structure in which the all-trans-retinal is covalently linked to the open form with a structure resembling the trans-Ret(HD)/open(MS) structure from our calculations.

The transformation from closed to open in Step 1 is caused by conformational changes responsible for activation (perhaps by the direct interaction of the trans-isomer with helix 3, to induce helix 3 to shift toward the extracellular side, breaking the DRY-associated salt bridges at the intracellular side).

Other processes hydrolyze off the trans-retinal to form a structure similar to Apo/open(MS) and then other processes reattach 11-cis-retinal to form a structure similar to Ret(HD)/open(MS).

The Ret(HD)/open(MS) relaxes eventually to form Ret(HD)/closed(MS), the inactive form. In this process the EC-II loop closes, perhaps caused by the helix 3 shifting toward the intracellular side, reforming the DRY-associated salt bridges at that end with the final result that the EC-II closes to form a structure similar to the inactive form.

Thus by using MembStruk and HierDock we have generated a total of six structures (summarized later) for ligand/protein complexes that can now be used to explore all the processes involving ligand binding and GPCR activation. The experiment provided just one of these six structures, but the validation with experiment allows us to have greater confidence in those five for which experimental structures are not available.

Using MembStruk we predicted the three-dimensional structure of bovine rhodopsin protein interacting with 11-cis-retinal using only primary sequence information. This led to the following structures.

Apo/closed(MS) is the MembStruk-predicted structure of the closed form, without the retinal. The transmembrane assembly for this structure deviates from Apo/closed(xtal) by 2.84 A CRMS for the main-chain atoms (4.04 Å CRMS for all transmembrane atoms, excluding H). Starting with the crystal structure and minimizing using the DREIDING FF leads to a structure that deviates from the crystal by 0.29 Å CRMS, indicating that the FF leads to a good description. Thus most of the 2.8 Å CRMS error is due to the MembStruk process. Ret(HD)/closed(MS) is the predicted structure for 11-cis-retinal obtained by applying HierDock to Apo/closed(MS). This leads to close contact (2.8 A) between the carbonyl of the retinal and the N of Lys-296. Forming the Schiff base linkage and minimizing leads to the Ret(HD) structure that deviates from Ret(HD)/closed(xtal) by 2.92 Å CRMS. Carrying out the same HierDock process for the minimized crystal structure leads to a predicted structure for 11-cis-retinal that deviates from Ret(xtal) by 0.62 Å CRMS. This indicates that it is, in fact, errors in the predicted protein structure that are responsible for the errors in ligand prediction.

Trans-Ret(HD)/closed(MS) is the predicted structure for all-trans-retinal obtained by converting 11-cis-retinal to all-trans and allowing the protein to respond. There is no experimental structure with which to compare.

Apo/open(MS) is the MembStruk-predicted structure of the open form without the retinal. There are no experiments with which to compare. This structure differs in the TM region from Apo/closed(MS) by 0.11 Å.

NoSB-Ret(HD)/open(MS) is the predicted structure for 11-cis-retinal obtained by applying HierDock to Apo/open(MS). There is no experimental structure with which to compare.

Ret(HD)/open(MS) is formed from NoSB-Ret(HD)/open(MS) by forming the Schiff base linkage to Lys-296 and minimizing. There are no experiments with which to compare. The retinal differs from that in Ret(HD)/closed(MS) by 1.74 A.

The validation with experiment is sufficiently good that we can now start to explore the mechanisms by carrying out long timescale molecular dynamics and Monte Carlo calculations on these various forms to learn more about the mechanism of activation. Comparisons of the structures and energetics for these systems provide information that might be useful for understanding the mechanisms of binding and activation in rhodopsin in particular and GPCRs in general.

We have noted above several steps for which we anticipate substantial improvements and we are continuing to improve the methods. For example the individual optimization of the helices can be performed under a more constrained environment by performing torsional dynamics of each helix in the presence of other helices or by performing torsional dynamics of all helices simultaneously. For improved accuracy in predicting the structures and for predicting the ligand binding energy, we also intend to take into account the differential solvent dielectric environment between membrane and the hydrophilic and interfacial dielectric constants (Spassov et al., 2002).

These applications of TM2ndS, MembStruk, and HierDock to bovine rhodopsin validate these techniques for predicting both the structure of membrane-bound proteins and the binding site of ligands to these proteins. The predictions from such studies can be used to design experiments to test details of the structures that might lead to improved structures. This could lead to structures more accurate than any of these techniques individually. The HierDock and MembStruk techniques validated here should also be useful for applications to other GPCRs, particularly for targeting agonists and antagonists against specific subtypes.

In addition, these studies open the door to examination of the mechanism for activation (structural and energy changes) of signaling. Obtaining independent structures for each of the major steps involved in binding and activation (e.g., the six structures discussed for retinal-rhodopsin) provides the basis for computational studies and for experiments that should provide a basis for detailed examination of each step.

References

Altenbach, C., K. Yang, D. L. Farrens, Z. T. Farahbakhsh, H. G. Khorana, and W. L. Hubbell. 1996. Structural features and light-dependent changes in the cytoplasmic interhelical E-F loop region of rhodopsin: a site-directed spin-labeling study. Biochemistry. 35: 12470-12478.

Altenbach, C., K. Cai, J. Kein-Seetharaman, H. G. Khorana, and W. L. Hubbell. 2001a. Structure and function in rhodopsin: mapping light-dependent changes in distance between residue 65 in helix TM 1 and residues in the sequence 306-319 at the cytoplasmic end of helix TM7 and in helix H8. Biochemistry. 40:15483-15492.

Altenbach, C., J. Klein-Seetharaman, K. Cai, H. G. Khorana, and W. L. Hubbell. 2001b. Structure and function in rhodopsin: mapping light-dependent changes in distance between residue 316 in helix 8 and residues in the sequence 60-75, covering the cytoplasmic end of helices TM1 and TM2 and their connection loop CL1. Biochemistry. 40:15493-15500.

Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Res. 25:3389-3402.

Altschul, S. F., W. Gish, W. Miller, W. E. Myers, and D. J. Lipman. 1990. Basic local alignment search tool. J. Mol. Biol. 215:403-410.

Archer, E., B. Maigret, C. Escrieut, L. Pradayrol, and D. Fourmy. 2003. Rhodopsin crystal: new template yielding realistic models of G-protein-coupled receptors? Trends Pharmacol. Sci. 24:36-40.

Borhan, B., M. L. Souto, H. Imai, Y. Schichida, and K. Nakanashi. 2000. Movement of retinal along the visual transduction path. Science. 288:2209-2212.

Bourne, H. R., and E. C. Meng. 2000. Structure-rhodopsin sees the light. Science. 289:733-734.

Bower, M., F. E. Cohen, and R. L. Dunbrack, Jr. 1997. Prediction of protein side-chain rotamers from a backbone-dependent rotamer library: a new homology modeling tool. J. Mol. Biol. 267:1268-1282.

Brameld, K. A., and W. A. Goddard. 1999. Ab initio quantum mechanical study of the structures and energies for the pseudorotation of 5′-dehydroxy analogues of 2′-deoxyribose and ribose sugars. J. Am. Chem. Soc. 121:985-993.

Cornette, J. L., K. B. Cease, H. Margalit, J. L. Spouge, J. A. Berzofsky, and C. Delisi. 1987. Hydrophobicity scales and computational techniques for detecting amphipathic structures in proteins. J. Mol. Biol. 195:659-685.

Datta, D., N. Vaidehi, X. Xu, and W. A. Goddard 3rd. 2002. Mechanism for antibody catalysis of the oxidation of water by singlet dioxygen. Proc. Natl. Acad. Sci. USA. 99:2636-2641.

Datta, D., N. Vaidehi, W. B. Floriano, K. S. Kim, N. V. Prasadarao, and W. A. Goddard. 2003. Interaction of E. coli outer-membrane protein A with sugars on the receptors of the brain microvascular endothelial cells. Proteins. 50:213-221.

Ding, H. Q., N. Karasawa, and W. A. Goddard. 1992. Atomic level simulations on a million particles—the cell multipole method for Coulomb and London nonbond interactions. Chem. Phys. Lett. 97:4309-4315.

Donnelly, D. 1993. Modeling alpha-helical transmembrane domains. Biochem. Soc.21:36-39.

Donnelly, D., J. P. Overington, and T. L. Blundell. 1994. The prediction and orientation of alpha-helices from sequence alignments-the combined use of environment-dependent substitution tables, Fourier-transform methods and helix capping rules. Protein Eng. 7:645-653.

Eisenberg, D., R. M. Weiss, and T. C. Terwilliger. 1984. The hydrophobic moment detects periodicity in protein hydrophobicity. Proc. Natl. Acad. Sci. USA. 8: 140-144.

Eisenberg, D., R. M. Weiss, T. C. Terwilliger, and W. Wilcox. 1982. Hydrophobic moments and protein structure. Faraday Symp. Chem. Soc. 17:109-120.

Ewing, T. A., and I. D. Kuntz. 1997. Critical evaluation of search algorithms for automated molecular docking and database screening. J. Comput. Chem. 18:1175-1189.

Farrens, D. L., C. Altenbach, K. Yang, W. L. Hubbell, and H. G. Khorana. 1996. Requirement of rigid-body motion of transmembrane helices for light activation of rhodopsin. Science. 274:768-770.

Floriano, W. B., N. Vaidehi, M. Singer, G. Shepherd, and W. A. Goddard 3rd. 2000. Molecular mechanisms underlying differential odor responses of a mouse olfactory receptor. Proc. Natl. Acad. Sci. USA. 97:10712-10716.

Floriano, W. B., N. Vaidehi, and W. A. Goddard 3rd. 2004a. Making sense of olfaction through molecular structure and function prediction of olfactory receptors. Chem. Senses. In press.

Floriano, W. B., N. Vaidehi, G. Zamanakos, and W. A. Goddard 3rd. 2004b. Virtual ligand screening of large molecule databases using hierarchical docking protocol (HierVLS). J. Med. Chem; 47:56-71.

Freddolino, P. L., M. Yashar, S. Kalani, N. Vaidehi, W. B. Floriano, S. E. Hall, R. J. Trabanino, V. W. T. Kam, and W. A. Goddard 3rd. 2004. Predicted 3D structure for the human β2 adrenergic receptor and its binding site for agonists and antagonists. PNAS. 101:2736-2741.

Greasley, P. J., F. Fanelli, O. Rossier, L. Abuin, and S. Cotecchia. 2002. Mutagenesis and modeling of the 1b-adrenergic receptor highlight the role of the helix 3/helix 6 interface in receptor activation. Mol. Pharma. 61:1025-1032.

Herzyk, P., and R. E. Hubbard. 1995. Automated method for modeling seven-helix transmembrane receptors from experimental data. Biophys. J. 69:2419-2442.

Jain, A., N. Vaidehi, and G. Rodriguez. 1993. Å fast recursive algorithm for molecular-dynamics simulation. J. Comput. Phys. 106:258-268.

Kalani, M. Y. S., N. Vaidehi, S. E. Hall, R. J. Trabanino, P. L. Freddolino, M. A. Kalani, W. B. Floriano, V. W. T. Kam, and W. A. Goddard 3rd. 2004. The predicted 3D structure of the human D2 dopamine receptor and the binding site and binding affinities for agonists and antagonists. PNAS. 103:3815-3820.

Kekenes-Huskey, P. M., N. Vaidehi, W. B. Floriano, and W. A. Goddard. 2003. Fidelity of phenyl alanyl tRNA synthetase in binding the natural amino acids. J. Chem. Phys. 107:11549-11557.

Laskowski, R. A., M. W. MacArthur, D. S. Moss, and J. M. Thornton. 1993. PROCHECK-a program to check the stereochemical quality of protein structures. J. Appl. Crystallogr. 26:283-291.

Lim, K. -T., S. Brunett, M. Iotov, R. B. McClurg, N. Vaidehi, S. Dasgupta, S. Taylor, and W. A. Goddard 3rd. 1997. Molecular dynamics for very large systems on massively parallel computers: the MPSim program. J. Comput. Chem. 18:501-521.

Lin, S. W., and T. P. Sakmar. 1996. Specific tryptophan UV-absorbance changes are probes of the transition of rhodopsin to its active state. Biochemistry. 35:11149-11159.

Lomize, A. L., I. D. Poghozeva, and H. I. Mosberg. 1999. Structural organization of G-protein-coupled receptors. J. Comp. Aided Mol. Design. 13:325-353.

MacKerell, A. D., D. Bashford, M. Bellott, R. L. Dunbrack, J. D. Evanseck, M. J. Field, S. Fischer, J. Gao, H. Guo, S. Ha, D. Joseph-McCarthy, I. Kuchnir, K. Kuczera, F. T. K. Lau, C. Mattos, S. Michnick, T. Ngo, D. T. Nguyen, B. Prodhom, W. E. Reiher, B. Roux, M. Schlenkrich, J. C. Smith, R. Stote, J. Straub, M. Watanabe, J. Wiorkiewicz-Kuczera, D. Yin, and M. Karplus. 1998. All-atom empirical potential for molecular modeling and dynamics studies of proteins. J. Phys. Chem. B. 102:3586-3616.

Malnic, B., J. Hirono, T. Sato, and L. B. Buck. 1999. Combinatorial receptor codes for odors. Cell. 96:713-723.

Marti-Renom, M. A., A. C. Stuart, A. Fiser, R. Sanchez, F. Melo, and A. Sali. 2000. Comparative protein structure modeling of genes and genomes. Annu. Rev. Biophys. Biomem. 29:291-325.

Mathiowetz, A. M., A. Jain, N. Karasawa, and W. A. Goddard 3rd. 1994. Protein simulations using techniques suitable for very large systems-the cell multipole method for nonbond interactions and the Newton-Euler inverse mass operator method for internal coordinate dynamics. Proteins. 20:227-247.

Mayo, S. L., B. D. Olafson, and W. A. Goddard 3rd. 1990. DREIDING-a generic force field for molecular simulations. J. Phys. Chem. 94:8897-8909.

Melia, T. J., C. W. Cowan, J. K. Angleson, and T. G. Wensel. 1997. Å comparison of the efficiency of G-protein activation by ligand-free and light-activated forms of rhodopsin. Biophys. J. 73:3182-3191.

Okada, T., O. P. Ernst, K. Palczewski, and K. P. Hofmann. 2001. Activation of rhodopsin: new insights from structural and biochemical studies. Trends Biochem. Sci. 26:318-324.

Palczewski, K., T. Kumasaka, T. Hori, C. Behnke, H. Motoshima, B. Fox, I. Trong, D. Teller, T. Okada, R. Stenkamp, M. Yamamoto, and M. Miyano. 2000. Crystal structure of rhodopsin: a G-protein-coupled receptor. Science. 289:739-745.

Rappe, A. K., and W. A. Goddard 3rd. 1991. Charge equilibration for molecular-dynamics simulations. J. Phys. Chem. 95:3358-3363.

Saam, J., E. Tajkhorshid, S. Hayashi, and K. Schulten. 2002. Molecular dynamics investigation of primary photoinduced events in the activation of rhodopsin. Biophys. J. 83:3097-3112.

Schertler, G. F. X. 1998. Structure of rhodopsin. Eye. 12:504-510.

Schöneberg, T., A. Schulz, and T. Gudermann. 2002. The structural basis of G-protein-coupled receptor function and dysfunction in human diseases. Rev. Phys. Biochem. Pharm. 144:145-227.

Shacham, S., M. Topf, N. Avisar, F. Glaser, Y. Marantz, S. Bar-Haim, S. Noiman, Z. Naor, and O. M. Becker. 2001. Modeling the three-dimensional structure of GPCRs from sequence. Med. Res. Rev. 21:472-483.

Spassov, V. Z., L. Yan, and S. Szalma. 2002. Introducing an implicit membrane in Generalized-Born solvent accessibility continuum solvent models. J. Phys. Chem. B. 106:8726-8738.

Strader, C. D., T. M. Fong, M. R. Tota, D. Underwood, and R. A. Dixon. 1994. Structure and function of G-protein-coupled receptors. Annu. Rev. Biochem. 63:101-132.

Strange, P. G. 1998. Three-state and two-state models. Trends Pharm. Sci. 19:85-86.

Thompson, J. D., D. G. Higgins, and T. J. Gibson. 1994. Clustal-W-improving the sensitivity of progressive multiple sequence alignment through sequence weighting, position-specific gap penalties and weight matrix choice. Nucleic Acids Res. 22:4673-4680.

Vaidehi, N., W. B. Floriano, R. Trabanino, S. E. Hall, P. Freddolino, E. J. Choi, G. Zamanakos, and W. A. Goddard. 2002. Prediction of structure and function of G-protein-coupled receptors. Proc. Natl. Acad. Sci. USA. 99:12622-12627.

Vaidehi, N., A. Jain, and W. A. Goddard 3rd. 1996. Constant temperature constrained molecular dynamics: the Newton-Euler inverse mass operator method. J. Phys. Chem. 100: 10508-10517.

Vriend, G. 1990. WHAT IF—a molecular modeling and drug design program. J. Mol. Graph. 8:52-56.

Wallin, E., and G. von Heijne. 1998. Genome-wide analysis of integral membrane proteins from eubacterial, archaean, and eukaryotic organisms. Protein Sci. 7:1029-1038.

Wang, P., N. Vaidehi, D. A. Tirrell, and W. A. Goddard 3rd. 2002. Virtual screening for binding of phenylalanine analogues to phenylalanyl-tRNA synthetase. J. Am. Chem. Soc. 124:14442-14449.

Wilson, S., and D. Bergsma. 2000. Orphan G-protein-coupled receptors: novel drug targets for the pharmaceutical industry. Drug Des. Discov. 17:105-114.

Zamanakos, G. 2002. Å fast and accurate analytical method for the computation of solvent effects in molecular simulations. Chemistry PhD thesis. California Institute of Technology, Pasadena, Calif.

The practice of the present invention will employ, unless otherwise indicated, conventional techniques of molecular biology, cell biology, cell culture, microbiology and recombinant DNA, which are within the skill of the art. Such techniques are explained fully in the literature. See, for example, Molecular Cloning: A Laboratory Manual, 2^{nd} Ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al.; U.S. Pat. No. 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.),

Immunochemical Methods In Cell And Molecular Biology (Mayer and Walker, eds., Academic Press, London, 1987).

The contents of all cited references (including literature references, issued patents, published patent applications as cited throughout this application) are hereby expressly incorporated by reference.

Equivalents

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

1. A method for predicting the structure of a transmembrane (TM) protein, comprising:
(1) identifying one or more TM regions from analysis of the primary sequence of said TM protein;
(2) assembling a TM bundle comprising all TM regions identified in (1), and optimizing the relative translation and/or rotational orientation of the TM regions in said TM bundle;
(3) optimizing each individual TM region in said TM bundle;
(4) fine-grain re-optimizing said TM bundle in explicit lipid bilayers;
(5) adding inter-TM region loops and side-chains for all amino acid residues to yield a full-atom model of said TM protein; and,
(6) optimizing said full-atom model and outputting a predicted structure therefor.

2. The method of claim 1, wherein said structure is a three-dimensional (3D) structure of the TM region(s) of said TM protein.

3. The method of claim 1, wherein said TM protein is a multipass TM protein.

4. The method of claim 3, wherein said multipass TM protein is an ion channel, a transporter, or a pump.

5. The method of claim 3, wherein said multipass TM protein has two or more TM regions.

6. The method of claim 3, wherein said multipass TM protein is a seven-TM protein.

7. The method of claim 6, wherein said seven-TM protein is a G Protein-Coupled Receptor (GPCR).

8. The method of claim 7, wherein said GPCR is an orphan GPCR or an olfactory receptor (OR).

9. The method of claim 7, wherein said GPCR is a rhodopsin-like receptor selected from: olfactory receptor, adenosine receptor, melanocortin receptor, biogenic amine receptor, vertebrate opsin, neuropeptide receptor, invertebrate opsin, chemokine receptor, chemotactic receptor, somatostatin receptor, opioid receptor, or melatonin receptor; a calcitonin and related receptor selected from: calcitonin receptor, calcitonin-like receptor, CRF receptor, PTH/PTHrP receptor, glucagon receptor, secretin receptor, or latrotoxin receptor; a metabotropic glutamate and related receptor selected from: metabotropic glutamate receptor, calcium receptor, GABA-B receptor, or putative pheromone receptor;
a STE2 pheromone receptor; a STE3 pheromone receptor; or cAMP receptor.

10. The method of claim 1, wherein said TM region comprises one or more potential α-helical region(s).

11. The method of claim 1, wherein said TM regions are TM helix regions.

12. The method of claim 11, wherein each of said TM helix region(s) comprises at least about 21 amino acid residues.

13. The method of claim 1, wherein step (1) is effectuated by a method based on hydrophobic profiling of at least a portion of said TM protein.

14. The method of claim 13, wherein said portion substantially excludes one or more of: the N- or C-terminal region(s) not in contact with lipid bilayers, or inter-TM region loops.

15. The method of claim 13, wherein said hydrophobic profiling uses peak signal analysis.

16. The method of claim 15, wherein said hydrophobic profiling uses the Eisenberg hydrophobicity scale.

17. The method of claim 16, wherein said TM regions are TM helix regions, said hydrophobic profiling uses the SeqHyd profile algorithm, and step (1) is effectuated by:
(1a) using the sequence of said protein as query, retrieving from a database an ensemble of hit sequences with 20-90% sequence identity, and/or BLAST bit score>200 and E-value>e⁻¹⁰⁰;
(1b) obtaining a multiple sequence alignment of said hit sequences and the sequence of said protein;
(1c) calculating consensus hydrophobicity for every residue position in said alignment, and plotting a hydrophobic profile;
(1d) assigning initial TM helix regions based on the global average hydrophobicity of said alignment, or a base_mod within 0.05 thereof; and,
(1e) capping each initial TM helix region identified in (1d) to yield a capped TM helix region, based on the presence of helix breakers.

18. The method of claim 17, wherein said database is a protein database, or a polynucleotide database translated in at least one of the six reading frames.

19. The method of claim 17, wherein said ensemble of hit sequences have a uniform distribution of sequences over the entire range of sequence identities.

20. The method of claim 19, wherein the lowest sequence identity to said protein within said ensemble of sequences is about 20-40%.

21. The method of claim 17, wherein said multiple sequence alignment is performed with ClustalW.

22. The method of claim 17, wherein step (1c) is performed with a window size (WS) between 12-20.

23. The method of claim 17, wherein step (1d) further comprising identifying additional TM region(s) with peak length<23 and peak area<0.8, using local average hydrophobicity more than 0.05 less than said base_mod, if said additional TM region(s) are not identified based either on said global average hydrophobicity or said base_mod.

24. The method of claim 17, wherein said helix breakers are independently selected from Pro (P), Gly (G), Arg (R), His (H), Lys (K), Glu (E), or Asp (D).

25. The method of claim 17, wherein said capped TM helix regions exclude N- and C-terminal helix breakers.

26. The method of claim 17, wherein the N- and C-termini of said capped TM helix regions are no more than 6 residues longer or 4 residues shorter than the N- and C-termini of said initial TM helix regions.

27. The method of claim 17, wherein each residue in each of said capped TM helix regions has an α-helical conformation.

28. The method of claim 27, wherein said α-helical conformation is characterized by a φ between −37 and −77 degrees, and a ψ between −27 and −67 degrees.

29. The method of claim 17, wherein said α-helical conformation is checked by verifying φ and ψ using PROCHECK.

30. The method of claim 1, wherein said protein is a GPCR, and in step (2), said TM bundle is assembled based on the 7.5 Å electron density map of frog rhodopsin.

31. The method of claim 1, wherein said protein is a GPCR, and in step (2), the relative translation of all helices in said TM bundle is optimized by placing the hydrophobicity center (HC) of each helix in the lipid midpoint plane (LMP).

32. The method of claim 1, wherein said protein is a GPCR, and in step (2), the rotational orientation of at least one helix in said TM bundle is optimized by the hydrophobic moment-based phobic orientation/CoarseRot-H.

33. The method of claim 32, wherein said phobic orientation depends on a hydrophobic midregion (HMR) defined by the middle 15 residues of each helix straddling the predicted HC.

34. The method of claim 32, wherein said at least one helix has significant contacts with a lipid membrane straddling the LMP.

35. The method of claim 1, wherein said protein is a GPCR, and in step (2), the rotational orientation of each of the at least one helix in said TM bundle is optimized individually by the energy minimization process RotMin.

36. The method of claim 35, wherein for each of the at least one helix, the RotMin comprises:
(a) designating one of said at least one helix as active helix;
(b) keeping the main chain of said active helix rigid while rotating said main-chain for a grid of rotation angles;
(c) optimizing side-chain positions of all residues for all helices in said TM bundle;
(d) minimizing energy for said active helix in the field of all other helices; and,
(e) repeating (a)-(d) for each of said at least one helix.

37. The method of claim 36, wherein said grid of rotation angles comprises a change of 2.5, 5, or 8 degrees over a range of +50 to +100 degrees.

38. The method of claim 36, wherein (c) is effectuated using SCWRL.

39. The method of claim 36, wherein (d) is effectuated by conjugate gradient minimization until an RMS force of 0.5 kcal/mol per A is achieved.

40. The method of claim 35, wherein said at least one helix is near the center of the GPCR TM barrel and not strongly amphipathic.

41. The method of claim 1, wherein said protein is a GPCR, and in step (2), the rotational orientation of all helices in said TM bundle is optimized by a combination of CoarseRot-H and RotMin.

42. The method of claim 1, wherein step (2) comprises generating an ensemble of assembled TM bundles by randomly combining permutations of favorable conformations of each TM region within the bundle, and screening for the most favored assembled TM bundle.

43. The method of claim 1, further comprising repeating the translation and/or rotational optimization in step (2) immediately after step (3).

44. The method of claim 1, wherein for each individual TM region, step (3) is effectuated by:
(a) placing all side-chains; and,
(b) minimizing energy using molecular dynamics (MD).

45. The method of claim 44, wherein step (a) is effectuated by SCWRL.

46. The method of claim 45, wherein step (b) is effectuated by simulations at 300 K for 500 ps, and minimizing the structure with the lowest potential energy in the last 250 ps using conjugate gradients.

47. The method of claim 45, wherein said molecular dynamics is Cartesian or torsional MD/NEIMO.

48. The method of claim 1, wherein said explicit lipid bilayers comprise molecules of dilauroylphosphatidylcholine lipid.

49. The method of claim 1, wherein step (4) is effectuated by quaternion-based rigid-body molecular dynamics (RB-MD) in MPSim.

50. The method of claim 49, wherein said MPSim is carried out for 50 ps or until the potential and kinetic energies of the system are stabilized.

51. The method of claim 1, wherein the loops are added in step (5) by WHATIF, and the side-chains for all amino acid residues are added by SCWRL.

52. The method of claim 1, wherein in step (5), the conformations of the loops are optimized by conjugate gradient minimization while keeping all TM regions fixed.

53. The method of claim 1, wherein step (5) further comprises adding selected disulfide bonds.

54. The method of claim 1, wherein step (6) is effectuated by full-atom conjugate gradient minimization in vacuum using MPSim.

55. The method of claim 1, further comprising verifying the predicted struture using HierDock.

56. A method for identifying a ligand specifically binding a GPCR, comprising:
(1) predicting the struture of said GPCR using the method of claim 1;
(2) identifying, amongst a plurality of candidate ligands, one or more ligands, if any, that specifically bind said GPCR using HierDock; and,
(3) verifying the binding specificity of each said one or more ligands to one or more other GPCRs;
wherein a preferential binding by said one or more ligands to said GPCR over said other GPCRs is indicative that said one or more ligands bind specifically to said GPCR.

57. The method of claim 56, wherein said GPCR is a target for a disease or condition.

58. The method of claim 56, wherein said GPCR is a mutant protein associate with a disease or condition.

59. The method of claim 56, wherein said ligand is an agonist of said GPCR.

60. The method of claim 56, wherein said ligand is an antagonist of said GPCR.

61. The method of claim 56, wherein step (3) is effectuated by HierDock.

62. The method of claim 61, wherein said one or more ligands bind to said GPCR with a minimal binding energy at least about 5-10 kcal/mol less than that for binding to said other GPCRs.

63. The method of claim 56, wherein step (3) is effectuated by biochemical measuring of ligand-receptor binding constant K_{D}.

64. The method of claim 63, wherein said one or more ligands bind to said GPCR with a K_{D} at least about 10-fold lower than that for binding to said other GPCRs.

65. Computer executable software code stored in a computer readable medium, which upon execution carries out a method for predicting the structure of a transmembrane (TM) protein, said method comprising:
(1) identifying one or more TM regions from analysis of the primary sequence of said TM protein;
(2) assembling a TM bundle comprising all identified TM regions, and optimizing the relative translation and/or rotational orientation of the TM regions in said bundle;
(3) optimizing each individual TM region in said TM bundle;
(4) fine-grain re-optimizing said TM bundle in explicit lipid bilayers;
(5) adding inter-TM region loops and side-chains for all amino acid residues to yield a full-atom model of said TM protein; and,
(6) optimizing said full-atom model and outputting a predicted structure therefor.

66. A system for predicting the structure of a transmembrane (TM) protein, comprising:
(1) a TM region identification module for identifying one or more TM regions from analysis of the primary sequence of said TM protein;
(2) a bundle assembly module for assembling a TM bundle comprising all TM regions identified in (1), and optimizing the relative translation and/or rotational orientation of the TM regions in said TM bundle;
(3) a TM region optimization module for optimizing each individual TM region in said TM bundle;
(4) a fine grain re-optimization module for fine-grain re-optimizing said TM bundle in explicit lipid bilayers;
(5) a full-atom model generation module for adding inter-TM region loops and side-chains for all amino acid residues to yield a full-atom model of said TM protein; and,
(6) a full-atom optimization module for optimizing said full-atom model and outputting a predicted structure therefor.

67. A computational model of the structure of a transmembrane protein, the computational model comprising: a computer-readable memory storing data describing an optimized predicted three-dimensional structure for the transmembrane protein, the optimized predicted structure being generated according to the method of claim 1.


SEQUENCE LISTING (27 sequences)

SEQ ID NO: 1 - protein, 8 aa, Bos taurus
EKEAYIHL

SEQ ID NO: 2 - protein, 12 aa, Bos taurus
LMLVEELCGACS

SEQ ID NO: 3 - protein, 10 aa, Bos taurus
RGNVFETMCI

SEQ ID NO: 4 - protein, 10 aa, Bos taurus
  VARIANT 6, 9, 10: Xaa = Any Amino Acid
FFSMTXDTXX

SEQ ID NO: 5 - protein, 23 aa, Bos taurus
FSMLAAYMFLLIMLGFPINFLTL

SEQ ID NO: 6 - protein, 32 aa, Bos taurus
PWQFSMLAAYMFLLIMLGFPINFLTLYVTVQH

SEQ ID NO: 7 - protein, 30 aa, Bos taurus
WQFSMLAAYMFLLIMLGFPINFLTLYVTVQ

SEQ ID NO: 8 - protein, 28 aa, Bos taurus
LNLAVADLFMVFGGFTTTLYTSLHGYFV

SEQ ID NO: 9 - protein, 31 aa, Bos taurus
PLNYILLNLAVADLFMVFGGFTTTLYTSLHG

SEQ ID NO: 10 - protein, 30 aa, Bos taurus
PLNYILLNLAVADLFMVFGGFTTTLYTSLH

SEQ ID NO: 11 - protein, 31 aa, Bos taurus
VFGPTGCNLEGFFATLGGEIALWSLVVLAIE

SEQ ID NO: 12 - protein, 28 aa, Bos taurus
PTGCNLEGFFATLGGEIALWSLVVLAIE

SEQ ID NO: 13 - protein, 33 aa, Bos taurus
PTGCNLEGFFATLGGEIALWSLVVLAIERYVVV

SEQ ID NO: 14 - protein, 20 aa, Bos taurus
IMGVAFTWVMALACAAPPLV

SEQ ID NO: 15 - protein, 23 aa, Bos taurus
HAIMGVAFTWVMALACAAPPLVG

SEQ ID NO: 16 - protein, 23 aa, Bos taurus
NHAIMGVAFTWVMALACAAPPLV

SEQ ID NO: 17 - protein, 25 aa, Bos taurus
VIYMFVVHFIIPLIVIFFCYGQLVF

SEQ ID NO: 18 - protein, 28 aa, Bos taurus
ESFVIYMFVVHFIIPLIVIFFCYGQLVF

SEQ ID NO: 19 - protein, 26 aa, Bos taurus
NESFVIYMFVVHFIIPLIVIFFCYGQ

SEQ ID NO: 20 - protein, 20 aa, Bos taurus
IIMVIAFLICWLPYAGVAFY

SEQ ID NO: 21 - protein, 7 aa, Bos taurus
KTSAVYN

SEQ ID NO: 22 - protein, 5 aa, Bos taurus
RYVVV

SEQ ID NO: 23 - protein, 5 aa, Bos taurus
GQLVF

SEQ ID NO: 24 - protein, 27 aa, Bos taurus
RMVIIMVIAFLICWLPYAGVAFYIFTH

SEQ ID NO: 25 - protein, 31 aa, Bos taurus
EKEVTRMVIIMVIAFLICWLPYAGVAFYIFT

SEQ ID NO: 26 - protein, 21 aa, Bos taurus
PIFMTIPAFFAKTSAVYNPVI

SEQ ID NO: 27 - protein, 21 aa, Bos taurus
IFMTIPAFFAKTSAVYNPVIY